SEQUENCE LISTING (42 sequences)

SEQ ID NO: 1 - protein, 9 aa, Artificial Sequence
  synthesized peptide moiety
RKKRRQRRR

SEQ ID NO: 2 - protein, 11 aa, Artificial Sequence
  synthesized peptide moiety
YARKARRQARR

SEQ ID NO: 3 - protein, 11 aa, Artificial Sequence
  synthesized peptide moiety
YARAARRAARR

SEQ ID NO: 4 - protein, 11 aa, Artificial Sequence
  synthesized peptide moiety
YARRRRRRRRR

SEQ ID NO: 5 - protein, 11 aa, Artificial Sequence
  synthesized peptide moiety
YAAARRRRRRR

SEQ ID NO: 6 - protein, 11 aa, Artificial Sequence
  synthesized peptide moiety
YARAPRRARRR

SEQ ID NO: 7 - protein, 10 aa, Artificial Sequence
  synthesized peptide moiety
YARAPRRPRR

SEQ ID NO: 8 - protein, 9 aa, Artificial Sequence
  synthesized peptide moiety
RKQKRRRRR

SEQ ID NO: 9 - protein, 9 aa, Artificial Sequence
  synthesized peptide moiety
RKKRQRRRR

SEQ ID NO: 10 - protein, 9 aa, Artificial Sequence
  synthesized peptide moiety
RKKRRRQRR

SEQ ID NO: 11 - protein, 9 aa, Artificial Sequence
  synthesized peptide moiety
RKKRRRRQR

SEQ ID NO: 12 - protein, 9 aa, Artificial Sequence
  synthesized peptide moiety
RKKRRRRRQ

SEQ ID NO: 13 - protein, 9 aa, Artificial Sequence
  synthesized peptide moiety
RKKQRRRRR

SEQ ID NO: 14 - protein, 9 aa, Artificial Sequence
  synthesized peptide moiety
RQKKRRRRR

SEQ ID NO: 15 - protein, 9 aa, Artificial Sequence
  synthesized peptide moiety
RQRRRRRRR

SEQ ID NO: 16 - protein, 10 aa, Artificial Sequence
  synthesized peptide moiety
RQRRRRRRRR

SEQ ID NO: 17 - protein, 9 aa, Artificial Sequence
  synthesized peptide moiety
RRQRRRRRR

SEQ ID NO: 18 - protein, 10 aa, Artificial Sequence
  synthesized peptide moiety
RRQRRRRRRR

SEQ ID NO: 19 - protein, 9 aa, Artificial Sequence
  synthesized peptide moiety
RRRQRRRRR

SEQ ID NO: 20 - protein, 10 aa, Artificial Sequence
  synthesized peptide moiety
RRRQRRRRRR

SEQ ID NO: 21 - protein, 9 aa, Artificial Sequence
  synthesized peptide moiety
RRRRQRRRR

SEQ ID NO: 22 - protein, 10 aa, Artificial Sequence
  synthesized peptide moiety
RRRRQRRRRR

SEQ ID NO: 23 - protein, 5 aa, Artificial Sequence
  synthesized peptide moiety
RRRRR

SEQ ID NO: 24 - protein, 9 aa, Artificial Sequence
  synthesized peptide moiety
RRRRRQRRR

SEQ ID NO: 25 - protein, 10 aa, Artificial Sequence
  synthesized peptide moiety
RRRRRQRRRR

SEQ ID NO: 26 - protein, 6 aa, Artificial Sequence
  synthesized peptide moiety
RRRRRR

SEQ ID NO: 27 - protein, 10 aa, Artificial Sequence
  synthesized peptide moiety
RRRRRRQRRR

SEQ ID NO: 28 - protein, 7 aa, Artificial Sequence
  synthesized peptide moiety
RRRRRRR

SEQ ID NO: 29 - protein, 10 aa, Artificial Sequence
  synthesized peptide moiety
RRRRRRRQRR

SEQ ID NO: 30 - protein, 8 aa, Artificial Sequence
  synthesized peptide moiety
RRRRRRRR

SEQ ID NO: 31 - protein, 10 aa, Artificial Sequence
  synthesized peptide moiety
RRRRRRRRQR

SEQ ID NO: 32 - protein, 9 aa, Artificial Sequence
  synthesized peptide moiety
RRRRRRRRR

SEQ ID NO: 33 - protein, 10 aa, Artificial Sequence
  synthesized peptide moiety
RRRRRRRRRQ

SEQ ID NO: 34 - protein, 9 aa, Artificial Sequence
  synthesized peptide moiety
QRKKRRRRR

SEQ ID NO: 35 - protein, 9 aa, Artificial Sequence
  synthesized peptide moiety
QRRRRRRRR

SEQ ID NO: 36 - protein, 10 aa, Artificial Sequence
  synthesized peptide moiety
QRRRRRRRRR

SEQ ID NO: 37 - protein, 11 aa, Artificial Sequence
  synthesized peptide moiety
YGRKKRRQRRR

SEQ ID NO: 38 - protein, 12 aa, Artificial Sequence
  synthesized peptide moiety
YGRKKRRQRRRX

SEQ ID NO: 39 - protein, 4 aa, Artificial Sequence
  synthesized peptide moiety
XXYG

SEQ ID NO: 40 - protein, 4 aa, Artificial Sequence
  synthesized peptide moiety
XXYG

SEQ ID NO: 41 - protein, 9 aa, Artificial Sequence
  synthesized peptide moiety
RKRRRQRRR

SEQ ID NO: 42 - protein, 9 aa, Artificial Sequence
  synthesized peptide moiety
RRKRRQRRR

(19) United States
(12) Patent Application Publication
(10) Pub. No.: US 2012/0135923 A1
(43) Pub. Date: May 31, 2012

(54) Use of Melanocortins to Treat Dyslipidemia

(75) Inventors: Heather A. Halem, Westborough, MA (US); Michael DeWitt Culler, Hopkinton, MA (US)

(73) Assignee: IPSEN PHARMA, S.A.S., Boulogne-Billancourt (FR)

(21) Appl. No.: 13/388,387

(22) PCT Filed: Jul. 30, 2010
(86) PCT No.: PCT/US2010/043832

§ 371 Date: Feb. 1, 2012

Related U.S. Application Data
(60) Provisional application No. 61/273,488, filed on Aug. 5, 2009.

(51) Int. Cl. A61K 38/08; A61K 38/10; A61P 3/06

(52) U.S. Cl. 514/7.4

(57) ABSTRACT

The present invention relates to peptide ligands of the melanocortin receptors, in particular the melancortin-4 receptor, and as such, are useful in the treatment of dyslipidemia and associated complications such as alcoholic and non-alcoholic fatty liver disease.

### BACKGROUND OF THE INVENTION

Melanocortins are a family of regulatory peptides which are formed by post-translational processing of pro-hormone pro-opiomelanocortin (POMC; 131 amino acids in length). POMC is processed into three classes of hormones; the melanocortins, adrenocorticotropin hormone, and various endorphins (e.g. lipotropin) (Cone et al., Recent Prog. Horm. Res., 51:287-317, (1996); Cone et al., Ann. N.Y. Acad. Sci., 31:342-363, (1993)).

Five melanocortin receptors (MC-R) have been characterized to date. These include melanocyte-specific receptor (MC1-R), corticoadrenal-specific ACTH receptor (MC2-R), melacortin-3 (MC3-R), melanocortin-4 (MC4-R) and melanocortin-5 receptor (MC5-R). All of the melanocortin receptors respond to the peptide hormone class of melanocyte stimulating hormones (MSH) (Cone et al., Ann. N.Y. Acad. Sci., 680:342-363 (1993); Cone et al., Recent Prog. Horm. Res., 51:287-318 (1996)).

There has been great interest in melanocortin (MC-R) receptors as targets for the design of novel therapeutics to treat disorders of body weight such as obesity and cachexia. One of the receptors, MC4-R, is a 332 amino acid transmembrane protein expressed in brain as well as placental and gut tissues (Cone et al., Ann. N.Y. Acad. Sci., 680:342-363 (1993); Cone et al., Recent Prog. Horm. Res., 51:287-318 (1996)). Recent pharmacological confirmation has established that central MC4-R receptors are the prime mediators of the anorexic and orexigenic effects reported for melanocortin agonists and antagonists, respectively (Giraudo et al., Brain Res., 809:302-306 (1998); Farooqi et al., NE J. Med., 348:1085-1095 (2003); MacNeil et al., Eu. J. Pharm., 44:141-157 (2002); MacNeil et al., Eu. J. Pharm., 450:93-109 (2002); Kask et al., NeuroReport, 10:707-711 (1999); Chen et al.,. Transgenic Res., 9:145-54, (2000); Marsh et al., Nat. Genet., 21:119-22, (1999); Balthasar et al., Cell, 123:493-505 (2005)).

In addition to the visible effects of body weight disorders, obese and overweight persons often develop a number of less-visible physiological complications such as diabetes, dyslipidemia, atherosclerosis, hypertension and hepatic steatosis. Hepatic steatosis may also affect persons considered to be normal or even underweight. Left unaddressed, heaptic steatosis can progress into fatty liver disease, inflammation of the liver, lesions, fibrosis and cancer. Concurrent with the rising occurrence of obesity, fatty liver disease is quickly becoming a global health problem for both adults and children (see Reddy et al., Am. J. Physiol. Gastrointest. Liver Physiol., 290:G852-858, (2006) and references therein).

### SUMMARY OF THE INVENTION

The present invention is directed to the use of peptides which are ligands of one or more of the melanocortin receptors (MC-R), or the pharmaceutically-acceptable salts thereof, to treat mammals suffering from dyslipidemia. In one embodiment, the ligands are agonists to the melanocortin 4 receptor. In a preferred embodiment, the melanocortin receptor ligands are according to the formulae described herein or are selected from particular peptides described herein.

The subject mammals suffering from dyslipidemia may be obese or overweight. The dyslipidemic subject mammals may also be normal weight or lean. In addition, the subject mammals may be human subjects of any age, such as an infant, a child, an adult or an elderly adult.

The subject mammals suffering from dyslipidemia by also suffer from increased levels of serum cholesterol, triglycerides, low-density lipoprotein cholesterol or free fatty acids or a decrease in high-density lipoprotein cholesterol concentration in the blood.

The subject mammals suffering from dyslipidemia may also suffer from hepatic steatosis. The hepatic steatosis may be non-alcoholic fatty acid liver disease or alcoholic fatty acid liver disease. The non-alcoholic fatty acid liver disease or alcoholic fatty acid liver disease may be accompanied by steatohepatitis, steatonecrosis, lobular inflammation, ballooning degeneration, fibrosis, cirrhosis or cancer or any combination thereof.

In the first embodiment, the invention provides a method to treat dyslipidemia in a mammalian subject by the administration of a therapeutically effective amount of a melanocortin receptor 4 ligand according to Formula (I) and pharmaceutically acceptable salts, hydrates, solvates or prodrugs thereof (see International Patent Application Publication Number WO 2007/008704, incorporated herein by reference in its entirety):

(R²R³)-A¹-c(A²-A³-A⁴-A⁵-A⁶-A⁷-A⁸-A⁹)-A¹⁰-R¹ (I)

wherein:

A¹ is Acc, HN—(CH₂)ₘ—C(O), L- or D-amino acid, or deleted;

A² is Cys, D-Cys, hCys, D-hCys, Pen, D-Pen, Asp, or Glu;

A³ is Gly, Ala, β-Ala, Gaba, Aib, D-amino acid, or deleted;

A⁴ is H is, 2-Pal, 3-Pal, 4-Pal, Taz, 2-Thi, 3-Thi, or (X¹,X²,X³,X⁴,X⁵)Phe;

A⁵ is D-Phe, D-1-Nal, D-2-Nal, D-Trp, D-Bal, D-(X¹,X²,X³,X⁴,X⁵)Phe, L-Phe or D-(Et)Tyr;

A⁶ is Arg, hArg, Dab, Dap, Lys, Orn, or HN—CH((CH₂)ₙ—N(R⁴R⁵))—C(O);

A⁷ is Trp, 1-Nal, 2-Nal, Bal, Bip, D-Trp, D-1-Nal, D-2-Nal, D-Bal or D-Bip;

A⁸ is Gly, D-Ala, Acc, Ala, β-Ala, Gaba, Apn, Ahx, Aha, HN—(CH₂)ₛ—C(O), or deleted;

A⁹ is Cys, D-Cys, hCys, D-hCys, Pen, D-Pen, Dab, Dap, Orn, or Lys;

A¹⁰ is Acc, HN—(CH₂)ₜ—C(O), L- or D-amino acid, or deleted;

R¹ is OH or NH₂;

each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;

each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;

m is, independently for each occurrence, 1, 2, 3, 4, 5, 6 or 7;

n is, independently for each occurrence, 1, 2, 3, 4 or 5;

s is, independently for each occurrence, 1, 2, 3, 4, 5, 6, or 7;

t is, independently for each occurrence, 1, 2, 3, 4, 5, 6, or 7;

X¹, X², X³, X⁴, and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN; provided that
(I). when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;
(II). when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
(III). either A³ or A⁸ or both must be present in said compound;
(IV). when A² is Cys, D-Cys, hCys, D-hCys, Pen, or D-Pen, then A⁹ is Cys, D-Cys, hCys, D-hCys, Pen, or D-Pen;
(V). when A² is Asp or Glu, then A⁹ is Dab, Dap, Orn, or Lys;
(VI). when A² is Ala or Gly, then A¹ is not Nle; and
(VII). when A¹ is deleted, then R² and R³ cannot both be H;
   or pharmaceutically acceptable salts thereof.

In one aspect of the first embodiment, the invention provides a method to treat dyslipidemia in a mammalian subject by the administration of a therapeutically effective amount of a subgroup of melanocortin receptor ligands of the immediate foregoing Formula I, wherein:
A¹ is A6c, Arg, D-Arg, Cha, D-Cha, hCha, Chg, D-Chg, Gaba, Ile, Leu, hLeu,
Met, β-hMet, 2-Nal, D-2-Nal, Nip, Nle, Oic, Phe, D-Phe, hPhe, hPro, Val, or deleted;
A² is Asp, Cys, D-Cys, hCys, D-hCys, Glu, Pen, or D-Pen;
A³ is D-Abu, Aib, Ala, β-Ala, D-Ala, D-Cha, Gaba, D-Glu, Gly, D-Ile, D-Leu, D-Tle, D-Val, or deleted;
A⁴ is H is or 3-Pal;
A⁵ is D-Bal, D-1-Nal, D-2-Nal, D-Phe, D-Trp, or D-(Et)Tyr;
A⁶ is Arg, or hArg;
A⁷ is Bal, Bip, 1-Nal, 2-Nal, Trp, D-Trp;
A⁸ is A6c, D-Ala, Aha, Ahx, Ala, β-Ala, Apn, Gaba, Gly or deleted;
A⁹ is Cys, D-Cys, hCys, D-hCys, Lys, Pen, or D-Pen;
A¹⁰ is Thr, or deleted;
wherein at least one of A³ or A⁸ is deleted, but not both,
or pharmaceutically acceptable salts thereof.

More preferred compounds of the immediately foregoing group of ligands according to Formula (I) useful to treat dyslipidemia in a mammalian subject are compounds of the formula:

### Ac-Arg-c(Cys-D-Ala-His-D-Phe-Arg-Trp-Cys)-NH₂;

### Ac-Nle-c(Asp-His-D-Phe-Arg-Trp-β-Ala-Lys)-NH₂;

Ac-Nle-c(Asp-His-D-Phe-Arg-Trp-A6c-Lys)-NH₂;

### Ac-Nle-c(Cys-His-D-Phe-Arg-Trp-Ahx-Cys)-NH₂;

### D-Phe-c(Cys-His-D-Phe-Arg-Trp-Ala-D-Cys)-Thr-NH₂;

### D-Phe-c(Cys-His-D-Phe-Arg-Trp-β-Ala-D-Cys)-Thr-NH₂;

### D-Phe-c(Cys-His-D-Phe-Arg-Trp-Gaba-D-Cys)-Thr-NH₂;

### Ac-Nle-c(Cys-His-D-Phe-Arg-Trp-Apn-Cys)-NH₂;

### Ac-Nle-c(Asp-His-D-Phe-Arg-Trp-Apn-Lys)-NH₂;

Ac-A6c-c(Asp-His-D-Phe-Arg-Trp-Gaba-Lys)-NH₂;

### Ac-D-2-Nal-c(Asp-His-D-Phe-Arg-Trp-Gaba-Lys)-NH₂;

### Ac-Cha-c(Asp-His-D-Phe-Arg-Trp-Gaba-Lys)-NH₂;

### Ac-Nle-c(Asp-His-D-Phe-Arg-Trp-Gaba-Lys)-NH₂;

### Ac-Nle-c(Cys-D-Ala-His-D-Phe-Arg-Trp-Cys)-NH₂;

### Ac-Nle-c(Cys-β-Ala-His-D-Phe-Arg-Trp-Cys)-NH₂;

### Ac-Nle-c(Cys-Gaba-His-D-Phe-Arg-Trp-Cys)-NH₂;

### Ac-Nle-c(Cys-Aib-His-D-Phe-Arg-Trp-Cys)-NH₂;

### Ac-Nle-c(Cys-Gly-His-D-Phe-Arg-Trp-Cys)-NH₂;

### Ac-Nle-c(D-Cys-Ala-His-D-Phe-Arg-Trp-Cys)-NH₂;

### Ac-Nle-c(D-Cys-D-Ala-His-D-Phe-Arg-Trp-Cys)-NH₂;

### Ac-Nle-c(D-Cys-β-Ala-His-D-Phe-Arg-Trp-Cys)-NH₂;

### Ac-Nle-c(D-Cys-Gaba-His-D-Phe-Arg-Trp-Cys)-NH₂;

### Ac-Nle-c(D-Cys-Aib-His-D-Phe-Arg-Trp-Cys)-NH₂;

### Ac-Nle-c(D-Cys-Gly-His-D-Phe-Arg-Trp-Cys)-NH₂;

### Ac-Nle-c(Cys-D-Ala-His-D-Phe-Arg-Trp-D-Cys)-NH₂;

### Ac-Nle-c(Cys-β-Ala-His-D-Phe-Arg-Trp-D-Cys)-NH₂;

### Ac-Nle-c(Cys-Gaba-His-D-Phe-Arg-Trp-D-Cys)-NH₂;

### Ac-Nle-c(Cys-Aib-His-D-Phe-Arg-Trp-D-Cys)-NH₂;

### Ac-Nle-c(Cys-Gly-His-D-Phe-Arg-Trp-D-Cys)-NH₂;

### Ac-Nle-c(D-Cys-Ala-His-D-Phe-Arg-Trp-D-Cys)-NH₂;

### Ac-Nle-c(D-Cys-D-Ala-His-D-Phe-Arg-Trp-D-Cys)-NH₂;

### Ac-Nle-c(D-Cys-β-Ala-His-D-Phe-Arg-Trp-D-Cys)-NH₂;

### Ac-Nle-c(D-Cys-Gaba-His-D-Phe-Arg-Trp-D-Cys)-NH₂;

### Ac-Nle-c(D-Cys-Aib-His-D-Phe-Arg-Trp-D-Cys)-NH₂;

### Ac-Oic-c(Asp-His-D-Phe-Arg-Trp-Gaba-Lys)-NH₂;

### Ac-Chg-c(Asp-His-D-Phe-Arg-Trp-Gaba-Lys)-NH₂;

### Ac-hCha-c(Asp-His-D-Phe-Arg-Trp-Gaba-Lys)-NH₂;

### Ac-D-Cha-c(Asp-His-D-Phe-Arg-Trp-Gaba-Lys)-NH₂;

### Ac-Nip-c(Asp-His-D-Phe-Arg-Trp-Gaba-Lys)-NH₂;

### Ac-hPro-c(Asp-His-D-Phe-Arg-Trp-Gaba-Lys)-NH₂;

### Ac-hLeu-c(Asp-His-D-Phe-Arg-Trp-Gaba-Lys)-NH₂;

### Ac-hCha-c(Asp-His-D-Phe-Arg-Trp-Gaba-Lys)-NH₂;

### Ac-Phe-c(Asp-His-D-Phe-Arg-Trp-Gaba-Lys)-NH₂;

### Ac-D-Phe-c(Asp-His-D-Phe-Arg-Trp-Gaba-Lys)-NH₂;

### Ac-D-Chg-c(Asp-His-D-Phe-Arg-Trp-Gaba-Lys)-NH₂;

n-butanoyl-Cha-c(Asp-His-D-Phe-Arg-Trp-Gaba-Lys)-NH₂;

### Ac-hPhe-c(Asp-His-D-Phe-Arg-Trp-Gaba-Lys)-NH₂;

Ac-β-hMet-c(Asp-His-D-Phe-Arg-Trp-Gaba-Lys)-NH₂;

### Ac-Gaba-c(Asp-His-D-Phe-Arg-Trp-Gaba-Lys)-NH₂;

### Ac-Cha-c(Asp-His-D-Phe-Arg-D-Trp-Ala-Lys)-NH₂;

### Ac-hCha-c(Asp-His-D-Phe-Arg-D-Trp-Ala-Lys)-NH₂;

### Ac-Leu-c(Asp-His-D-Phe-Arg-D-Trp-Ala-Lys)-NH₂;

### Ac-hLeu-c(Asp-His-D-Phe-Arg-D-Trp-Ala-Lys)-NH₂;

### Ac-Phe-c(Asp-His-D-Phe-Arg-D-Trp-Ala-Lys)-NH₂;

### Ac-Nle-c(Asp-His-D-Phe-Arg-D-Trp-D-Ala-Lys)-NH₂;

### Ac-Nle-c(Asp-His-D-Phe-Arg-D-Trp-β-Ala-Lys)-NH₂;

### Ac-Nle-c(Asp-His-D-Phe-Arg-D-Trp-Gaba-Lys)-NH₂;

### Ac-Nle-c(Asp-His-D-Phe-Arg-D-Trp-Aha-Lys)-NH₂;

### Ac-Nle-c(Asp-His-D-Phe-Arg-D-Trp-Apn-Lys)-NH₂;

### Ac-Nle-c(Cys-His-D-Phe-Arg-D-Trp-Apn-Cys)-NH₂;

### Ac-Nle-c(Cys-His-D-Phe-Arg-D-Trp-Gaba-Cys)-NH₂;

### Ac-Nle-c(Cys-His-D-Phe-Arg-D-Trp-Ahx-Cys)-NH₂;

### Ac-Nle-c(Cys-His-D-Phe-Arg-D-Trp-β-Ala-Cys)-NH₂;

### Ac-Nle-c(Cys-His-D-Phe-Arg-D-Trp-D-Ala-Cys)-NH₂;

### Ac-Nle-c(Cys-D-Ala-His-D-2-Nal-Arg-Trp-Cys)-NH₂;

### Ac-Nle-c(Cys-D-Ala-His-D-2-Nal-Arg-2-Nal-Cys)-NH₂;

### Ac-Nle-c(Cys-D-Ala-His-D-2-Nal-Arg-1-Nal-Cys)-NH₂;

n-butanoyl-Nle-c(Cys-D-Ala-His-D-Phe-Arg-2-Nal-Cys)-NH₂;
n-butanoyl-Nle-c(Cys-D-Ala-His-D-Phe-Arg-Trp-Cys)-NH₂;

### Ac-Nle-c(Cys-D-Ala-His-D-Phe-Arg-2-Nal-Cys)-NH₂;

### Ac-Nle-c(Cys-D-Ala-His-D-Phe-Arg-1-Nal-Cys)-NH₂;

### Ac-Nle-c(Cys-D-Ala-His-D-Phe-Arg-Bal-Cys)-NH₂;

### Ac-Nle-c(Cys-D-Glu-His-D-Phe-Arg-Trp-Cys)-NH₂;

### Ac-Nle-c(Asp-His-D-Phe-Arg-Trp-D-Ala-Lys)-NH₂;

### Ac-Nle-c(Cys-D-Ala-His-D-2-Nal-Arg-Bal-Cys)-NH₂;

### Ac-Nle-c(Pen-D-Ala-His-D-Phe-Arg-Trp-Cys)-NH₂;

### Ac-Nle-c(Cys-D-Ala-His-D-Phe-Arg-Trp-Pen)-NH₂;

### Ac-Nle-c(Pen-D-Ala-His-D-Phe-Arg-Trp-Pen)-NH₂;

### D-Phe-c(Cys-His-D-Phe-hArg-Trp-β-Ala-D-Cys)-Thr-NH₂;

### D-Phe-c(Cys-His-D-(Et)Tyr-Arg-Trp-β-Ala-D-Cys)-Thr-NH₂;

### D-Phe-c(Cys-His-D-Phe-Arg-Bip-β-Ala-D-Cys)-Thr-NH₂;

### D-Phe-c(Cys-His-D-(Et)Tyr-hArg-Trp-β-Ala-D-Cys)-Thr-NH₂;

### D-Phe-c(Cys-His-D-Phe-hArg-Bip-β-Ala-D-Cys)-Thr-NH₂;

### D-Phe-c(Cys-His-D-(Et)Tyr-hArg-Bip-β-Ala-D-Cys)-Thr-NH₂;

### Nle-c(Cys-His-D-Phe-Arg-Trp-Apn-Cys)-NH₂;

### Ac-Nle-c(Asp-D-Ala-His-D-Phe-Arg-Trp-Lys)-NH₂;

### Ac-Nle-c(Asp-D-Ala-His-D-Phe-Arg-Bal-Lys)-NH₂;

### Ac-Nle-c(Cys-D-Ala-His-D-Phe-Arg-Trp-Pen)-OH;

### Ac-Nle-c(Cys-D-Abu-His-D-Phe-Arg-Trp-Cys)-NH₂;

### Ac-Nle-c(Cys-D-Val-His-D-Phe-Arg-Trp-Cys)-NH₂;

### Ac-Nle-c(Cys-D-Ile-His-D-Phe-Arg-Trp-Cys)-NH₂;

### Ac-Nle-c(Cys-D-Leu-His-D-Phe-Arg-Trp-Cys)-NH₂;

### Ac-Nle-c(Cys-D-Tle-His-D-Phe-Arg-Trp-Cys)-NH₂;

### Ac-Nle-c(Cys-D-Cha-His-D-Phe-Arg-Trp-Cys)-NH₂;

### Ac-Nle-c(Pen-His-D-Phe-Arg-Trp-Gaba-Cys)-NH₂;

### Ac-Nle-c(Cys-His-D-Phe-Arg-Trp-Gaba-Pen)-NH₂;

### Ac-Nle-c(Pen-His-D-Phe-Arg-Trp-Gaba-Pen)-NH₂;

### Ac-Leu-c(Cys-His-D-Phe-Arg-Trp-Gaba-Cys)-NH₂;

### Ac-Cha-c(Cys-His-D-Phe-Arg-Trp-Gaba-Cys)-NH₂;

### Ac-Ile-c(Cys-His-D-Phe-Arg-Trp-Gaba-Cys)-NH₂;

### Ac-Phe-c(Cys-His-D-Phe-Arg-Trp-Gaba-Cys)-NH₂;

### Ac-Val-c(Cys-His-D-Phe-Arg-Trp-Gaba-Cys)-NH₂;

### Ac-2-Nal-c(Cys-His-D-Phe-Arg-Trp-Gaba-Cys)-NH₂;

### Nle-c(Cys-His-D-Phe-Arg-Trp-Gaba-Cys)-NH₂;

### Phe-c(Cys-His-D-Phe-Arg-Trp-Gaba-Cys)-NH₂;

### Ac-Nle-c(Cys-3-Pal-D-Phe-Arg-Trp-Gaba-Cys)-NH₂;

### Ac-Nle-c(Cys-D-Ala-His-D-Phe-Arg-Trp-Cys)-OH;

### Ac-Nle-c(Cys-His-Phe-Arg-D-Trp-Gaba-Cys)-NH₂;

### Ac-Nle-c(Asp-His-D-2-Nal-Arg-Trp-Ala-Lys)-NH₂;

### Ac-Nle-c(Asp-His-D-2-Nal-Arg-Trp-β-Ala-Lys)-NH₂;

### Ac-Nle-c(Cys-His-D-2-Nal-Arg-Trp-Gaba-Cys)-NH₂;

### Ac-Nle-c(Cys-His-D-2-Nal-Arg-Trp-Ahx-Cys)-NH₂;

### Ac-hPhe-c(Asp-His-D-2-Nal-Arg-Trp-Gaba-Lys)-NH₂;

### Ac-Cha-c(Asp-His-D-2-Nal-Arg-Trp-Gaba-Lys)-NH₂;

### Ac-Nle-c(Asp-His-D-Phe-Arg-Trp-β-Ala-Lys)—OH;

### Ac-Nle-c(Cys-His-D-Phe-Arg-Trp-Ahx-Cys)-OH;

### D-Phe-c(Cys-His-D-Phe-Arg-Trp-Ala-D-Cys)-Thr-OH;

### D-Phe-c(Cys-His-D-Phe-Arg-Trp-β-Ala-D-Cys)-Thr-OH;

### D-Phe-c(Cys-His-D-Phe-Arg-Trp-Gaba-D-Cys)-Thr-OH;

### Ac-Nle-c(Cys-His-D-Phe-Arg-Trp-Apn-Cys)-OH;

### Ac-Nle-c(Asp-His-D-Phe-Arg-Trp-Apn-Lys)-OH;

### Ac-Cha-c(Asp-His-D-Phe-Arg-Trp-Gaba-Lys)-OH;

### Ac-Nle-c(Asp-His-D-Phe-Arg-Trp-Gaba-Lys)—OH;

### Ac-Nle-c(Cys-D-Ala-His-D-Phe-Arg-Trp-Cys)-OH;

### Ac-Chg-c(Asp-His-D-Phe-Arg-Trp-Gaba-Lys)-OH;

### Ac-D-Cha-c(Asp-His-D-Phe-Arg-Trp-Gaba-Lys)—OH;

### Ac-hCha-c(Asp-His-D-Phe-Arg-Trp-Gaba-Lys)—OH;

### Ac-D-Chg-c(Asp-His-D-Phe-Arg-Trp-Gaba-Lys)-OH;

### Ac-hPhe-c(Asp-His-D-Phe-Arg-Trp-Gaba-Lys)—OH;

### Ac-Nle-c(Cys-His-D-Phe-Arg-D-Trp-Gaba-Cys)-OH;

### Ac-Nle-c(Cys-His-D-Phe-Arg-D-Trp-Ahx-Cys)-OH;

### Ac-Nle-c(Cys-His-D-Phe-Arg-D-Trp-β-Ala-Cys)—OH;

### Ac-Nle-c(Cys-His-D-Phe-Arg-D-Trp-D-Ala-Cys)-OH;

### Ac-Nle-c(Cys-D-Ala-His-D-2-Nal-Arg-Trp-Cys)-OH;

### Ac-Nle-c(Cys-D-Ala-His-D-2-Nal-Arg-2-Nal-Cys)—OH;

### Ac-Nle-c(Cys-D-Ala-His-D-2-Nal-Arg-1-Nal-Cys)—OH;

### Ac-Nle-c(Cys-D-Ala-His-D-2-Nal-Arg-Bal-Cys)-OH;

### Ac-Nle-c(Pen-D-Ala-His-D-Phe-Arg-Trp-Cys)-OH;

### Ac-Nle-c(Cys-D-Ala-His-D-Phe-Arg-Trp-Pen)-OH;

### Ac-Nle-c(Cys-His-D-Phe-Arg-Trp-Gaba-Pen)-OH;

### Ac-Arg-c(Cys-D-Ala-His-D-2-Nal-Arg-Trp-Cys)-NH₂;

### Ac-D-Arg-c(Cys-D-Ala-His-D-Phe-Arg-Trp-Cys)-NH₂;

### Ac-D-Arg-c(Cys-D-Ala-His-D-Phe-Arg-Trp-Pen)-NH₂;

### Ac-D-Arg-c(Cys-His-D-Phe-Arg-Trp-Gaba-Pen)-NH₂;

### Ac-Arg-c(Cys-His-D-Phe-Arg-Trp-Gaba-Pen)-NH₂;

### Ac-Arg-c(Cys-D-Ala-His-D-Phe-Arg-Trp-Pen)-NH₂;

### Ac-D-Arg-c(Asp-His-D-Phe-Arg-Trp-Ala-Lys)-NH₂;

### Ac-Arg-c(Asp-His-D-Phe-Arg-Trp-Ala-Lys)-NH₂;

### Ac-Nle-c(Cys-D-Ala-His-D-Phe-Arg-Trp-Cys)-NH₂;

### D-Phe-c(Cys-His-D-(Et)Tyr-Arg-Trp-β-Ala-D-Cys)-Thr-NH₂;

### Nle-c(Cys-His-D-Phe-Arg-Trp-Apn-Cys)-NH₂;

### Ac-Nle-c(Cys-His-D-Phe-Arg-Trp-Gaba-Pen)-NH₂;

### Nle-c(Cys-His-D-Phe-Arg-Trp-Gaba-Cys)-NH₂;

### Ac-Nle-c(Asp-His-D-Phe-Arg-Trp-β-Ala-Lys)-NH₂;

### Ac-Nle-c(Cys-His-D-Phe-Arg-Trp-Ahx-Cys)-NH₂;

### D-Phe-c(Cys-His-D-Phe-Arg-Trp-β-Ala-D-Cys)-Thr-NH₂;

### D-Phe-c(Cys-His-D-Phe-Arg-Trp-Gaba-D-Cys)-Thr-NH₂;

### Ac-Nle-c(Cys-His-D-Phe-Arg-Trp-Apn-Cys)-NH₂;

### Ac-Nle-c(Asp-His-D-Phe-Arg-Trp-Apn-Lys)-NH₂;

### Ac-Cha-c(Asp-His-D-Phe-Arg-Trp-Gaba-Lys)-NH₂;

### Ac-Nle-c(Asp-His-D-Phe-Arg-Trp-Gaba-Lys)-NH₂;

### Ac-Chg-c(Asp-His-D-Phe-Arg-Trp-Gaba-Lys)-NH₂;

### Ac-hCha-c(Asp-His-D-Phe-Arg-Trp-Gaba-Lys)-NH₂;

### Ac-D-Chg-c(Asp-His-D-Phe-Arg-Trp-Gaba-Lys)-NH₂;

### Ac-hPhe-c(Asp-His-D-Phe-Arg-Trp-Gaba-Lys)-NH₂;

### Ac-Nle-c(Cys-His-D-Phe-Arg-D-Trp-β-Ala-Cys)-NH₂;

### Ac-Nle-c(Pen-D-Ala-His-D-Phe-Arg-Trp-Cys)-NH₂;

### Ac-Nle-c(Cys-D-Ala-His-D-Phe-Arg-Trp-Pen)-NH₂;

### D-Phe-c(Cys-His-D-Phe-hArg-Trp-β-Ala-D-Cys)-Thr-NH₂;

### D-Phe-c(Cys-His-D-Phe-Arg-Bip-β-Ala-D-Cys)-Thr-NH₂;

### D-Phe-c(Cys-His-D-(Et)Tyr-hArg-Trp-β-Ala-D-Cys)-Thr-NH₂;

### D-Phe-c(Cys-His-D-Phe-hArg-Bip-β-Ala-D-Cys)-Thr-NH₂;

### D-Phe-c(Cys-His-D-(Et)Tyr-hArg-Bip-β-Ala-D-Cys)-Thr-NH₂;

### Ac-Nle-c(Asp-D-Ala-His-D-Phe-Arg-Trp-Lys)-NH₂;

### Ac-Nle-c(Asp-D-Ala-His-D-Phe-Arg-Bal-Lys)-NH₂;

### Ac-Nle-c(Cys-D-Ala-His-D-Phe-Arg-Trp-Pen)-OH;

### Ac-Nle-c(Cys-D-Abu-His-D-Phe-Arg-Trp-Cys)-NH₂;

### Ac-Nle-c(Cys-D-Val-His-D-Phe-Arg-Trp-Cys)-NH₂;

### Ac-Nle-c(Cys-D-Ile-His-D-Phe-Arg-Trp-Cys)-NH₂;

### Ac-Nle-c(Cys-D-Leu-His-D-Phe-Arg-Trp-Cys)-NH₂;

### Ac-Nle-c(Cys-D-Tle-His-D-Phe-Arg-Trp-Cys)-NH₂;

### Ac-Nle-c(Cys-D-Cha-His-D-Phe-Arg-Trp-Cys)-NH₂;

### Ac-Nle-c(Pen-His-D-Phe-Arg-Trp-Gaba-Cys)-NH₂;

### Ac-Nle-c(Pen-His-D-Phe-Arg-Trp-Gaba-Pen)-NH₂;

### Ac-Leu-c(Cys-His-D-Phe-Arg-Trp-Gaba-Cys)-NH₂;

### Ac-Cha-c(Cys-His-D-Phe-Arg-Trp-Gaba-Cys)-NH₂;

### Ac-Ile-c(Cys-His-D-Phe-Arg-Trp-Gaba-Cys)-NH₂;

### Ac-Phe-c(Cys-His-D-Phe-Arg-Trp-Gaba-Cys)-NH₂;

### Ac-Val-c(Cys-His-D-Phe-Arg-Trp-Gaba-Cys)-NH₂;

### Ac-2-Nal-c(Cys-His-D-Phe-Arg-Trp-Gaba-Cys)-NH₂;

Phe-c(Cys-His-D-Phe-Arg-Trp-Gaba-Cys)-NH₂;

### Ac-Nle-c(Cys-3-Pal-D-Phe-Arg-Trp-Gaba-Cys)-NH₂;

### Ac-Nle-c(Cys-D-Ala-His-D-Phe-Arg-Trp-Cys)-OH;

### Ac-Nle-c(Cys-His-Phe-Arg-D-Trp-Gaba-Cys)-NH₂;

### Ac-Nle-c(Cys-D-Ala-His-D-2-Nal-Arg-Trp-Cys)-NH₂;

### Ac-Nle-c(Cys-D-Ala-His-D-2-Nal-Arg-2-Nal-Cys)-NH₂;

### Ac-Nle-c(Cys-D-Ala-His-D-2-Nal-Arg-Bal-Cys)-NH₂;

### Ac-Nle-c(Asp-His-D-2-Nal-Arg-Trp-Ala-Lys)-NH₂;

### Ac-Nle-c(Asp-His-D-2-Nal-Arg-Trp-β-Ala-Lys)-NH₂;

### Ac-Nle-c(Cys-His-D-2-Nal-Arg-Trp-Gaba-Cys)-NH₂;

### Ac-Nle-c(Cys-His-D-2-Nal-Arg-Trp-Ahx-Cys)-NH₂;

### Ac-hPhe-c(Asp-His-D-2-Nal-Arg-Trp-Gaba-Lys)-NH₂;

### Ac-Cha-c(Asp-His-D-2-Nal-Arg-Trp-Gaba-Lys)-NH₂; or

### Ac-Arg-c(Cys-D-Ala-His-D-2-Nal-Arg-Trp-Cys)-NH₂;

or pharmaceutically acceptable salts thereof.

In the second embodiment, the invention provides a method to treat dyslipidemia in a mammalian subject by the administration of a therapeutically effective amount of a melanocortin receptor ligand according to Formula (II) and pharmaceutically acceptable salts, hydrates, solvates or prodrugs thereof (see International Patent Application Publication Number WO 2007/008704 incorporated herein by reference in its entirety):

(R²R³)-A¹-c(A²-A³-A⁴-A⁵-A⁶-A⁷-A⁸-A⁹)NH₂ (II)

wherein:

A¹ is Nle or deleted;

A² is Cys or Asp;

A³ is Glu or D-Ala;

A⁴ is H is;

A⁵ is D-Phe;

A⁶ is Arg;

A⁷ is Trp, 2-Nal or Bal;

A⁸ is Gly, Ala, D-Ala, β-Ala, Gaba or Apn;

A⁹ is Cys or Lys;

each of R² and R³ is independently selected from the group consisting of H or (C₁-C₆)acyl;

provided that

(I). when R² is (C₁-C₆)acyl, then R³ is H; and

(II). when A² is Cys, then A⁹ is Cys,

or a pharmaceutically acceptable salt thereof.

More preferred of the immediately foregoing group of compounds which are useful to dyslipidemia in a mammalian subject are compounds of the formula:

### Ac-Nle-c(Cys-D-Ala-His-D-Phe-Arg-Trp-Gly-Cys)-NH₂;

### Ac-Nle-c(Cys-D-Ala-His-D-Phe-Arg-Trp-D-Ala-Cys)-NH₂;

### Ac-Nle-c(Cys-D-Ala-His-D-Phe-Arg-Trp-β-Ala-Cys)-NH₂;

### Ac-Nle-c(Cys-D-Ala-His-D-Phe-Arg-Trp-Gaba-Cys)-NH₂;

### Ac-Nle-c(Cys-D-Ala-His-D-Phe-Arg-Trp-Apn-Cys)-NH₂;

### Ac-c(Cys-Glu-His-D-Phe-Arg-Trp-Ala-Cys)-NH₂;

### Ac-c(Cys-Glu-His-D-Phe-Arg-2-Nal-Ala-Cys)-NH₂;

### Ac-c(Cys-D-Ala-His-D-Phe-Arg-Trp-Ala-Cys)-NH₂;

### Ac-c(Cys-D-Ala-His-D-Phe-Arg-2-Nal-Ala-Cys)-NH₂;

### Ac-Nle-c(Cys-D-Ala-His-D-Phe-Arg-Trp-Ala-Cys)-NH₂;

### Ac-Nle-c(Cys-D-Ala-His-D-Phe-Arg-Trp-(3-Ala-Cys)-NH₂;

### Ac-Nle-c(Cys-D-Ala-His-D-Phe-Arg-Trp-Gaba-Cys)-NH₂; or

### Ac-Nle-c(Asp-D-Ala-His-D-Phe-Arg-Bal-Ala-Lys)-NH₂;

or a pharmaceutically acceptable salt thereof.

In the third embodiment, the invention provides a method to treat dyslipidemia in a mammalian subject by the administration of a therapeutically effective amount of a melanocortin receptor compound according to Formula (III), and pharmaceutically acceptable salts, hydrates, solvates or prodrugs thereof (see International Application Publication Number WO 2007/008684, incorporated herein by reference in its entirety):

(R²R³)-B¹-A¹-c(A²-A³-A⁴-A⁵-A⁶-A⁷-A⁸-A⁹)-A¹⁰-A¹¹-A¹²-A¹³-B²-B³-R¹ (III)

wherein:

B¹ is a peptide moiety which contains 5, 6, 7, 8, 9, 10, 11, 12, 13, 14, or 15 amino acids, wherein at least 5 amino acids are independently selected from the group consisting of L-Arg, D-Arg, L-hArg and D-hArg, or B¹ is optionally deleted;

A¹ is Acc, HN—(CH₂)ₘ—C(O), L- or D-amino acid or deleted;

A² is Cys, D-Cys, hCys, D-hCys, Pen, D-Pen, Asp or Glu;

A³ is Gly, Glu, Ala, β-Ala, Gaba, Aib, D-amino acid or deleted;

A⁴ is H is, 2-Pal, 3-Pal, 4-Pal, Taz, 2-Thi, 3-Thi or (X¹,X²,X³,X⁴,X⁵)Phe;

A⁵ is D-Phe, D-1-Nal, D-2-Nal, D-Trp, D-Bal, D-(X¹,X²,X³,X⁴,X⁵)Phe, D-(Et)Tyr, D-Dip, D-Bip or D-Bpa;

A⁶ is Arg, hArg, Dab, Dap, Lys, Orn or HN—CH((CH₂)ₙ—N(R⁴R⁵))—C(O);

A⁷ is Trp, 1-Nal, 2-Nal, Bal, Bip, Dip, Bpa, D-Trp, D-1-Nal, D-2-Nal, D-Bal, D-Bip, D-Dip or D-Bpa;

A⁸ is Gly, D-Ala, Acc, Ala, β-Ala, Gaba, Apn, Ahx, Aha, HN—(CH₂)ₛ—C(O) or deleted;

A⁹ is Cys, D-Cys, hCys, D-hCys, Pen, D-Pen, Dab, Dap, Orn or Lys;

A¹⁰ is Acc, HN—(CH₂)ₜ—C(O), Pro, hPro, 3-Hyp, 4-Hyp, Thr, an L- or D-amino acid or deleted;

A¹¹ is Pro, hPro, 3-Hyp, 4-Hyp or deleted;

A¹² is Lys, Dab, Dap, Arg, hArg or deleted;

A¹³ is Asp, Glu or deleted;

B² is a peptide moiety containing 1, 2, 3, 4, or 5 amino acids or deleted,

B³ is a peptide moiety which contains 5, 6, 7, 8, 9, 10, 11, 12, 13, 14 or 15 amino acids wherein at least 5 amino acids are independently selected from the group consisting of L-Arg, D-Arg, L-hArg and D-hArg, or is deleted;

R¹ is OH or NH₂;

R² and R³ each is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl and substituted aryl(C₁-C₃₀)acyl;

R⁴ and R⁵ each is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl or C(NH)—NH₂;

n is, independently for each occurrence, 1, 2, 3, 4 or 5;

m is, independently for each occurrence, 1, 2, 3, 4, 5, 6 or 7;

s is, independently for each occurrence, 1, 2, 3, 4, 5, 6 or 7;

t is, independently for each occurrence, 1, 2, 3, 4, 5, 6 or 7;

X¹, X², X³, X⁴ and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂ or CN; provided that:

(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl or C(NH)—NH₂, then R⁵ is H, (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl or substituted aryl(C₁-C₄₀)alkyl;

(II) when R² is (C₁-C₃₀)acyl, aryl(C₁-C₃₀)acyl, substituted (C₁-C₃₀)acyl or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted

(C₁-C₃₀)heteroalkyl, substituted (C₂-C₃₀)alkenyl, substituted (C₂-C₃₀)alkynyl or substituted aryl(C₁-C₃₀)alkyl;

(III) neither B¹ nor B² contains one or more of the following amino acid sequences: Arg-(Lys)₂-(Arg)₂-Gln-(Arg)₃ (SEQ ID NO:1), Tyr-Ala-Arg-Lys-Ala-(Arg)₂-Gln-Ala-(Arg)₂ (SEQ ID NO:2), Tyr-Ala-Arg-(Ala)₂-(Arg)₂-(Ala)₂-(Arg)₂ (SEQ ID NO:3), Tyr-Ala-(Arg)₉ (SEQ ID NO:4), Tyr-(Ala)₃-(Arg)₇ (SEQ ID NO:5), Tyr-Ala-Arg-Ala-Pro-(Arg)₂-Ala-(Arg)₃ (SEQ ID NO:6) or Tyr-Ala-Arg-Ala-Pro-(Arg)₂-Pro-(Arg)₂ (SEQ ID NO:7);

(IV) either B¹ or B² or both must be present in said compound;

(V) when A² is Cys, D-Cys, hCys, D-hCys, Pen or D-Pen, then A⁹ is Cys, D-Cys, hCys, D-hCys, Pen or D-Pen; and

(VI) when A² is Asp or Glu, then A⁹ is Dab, Dap, Orn or Lys;

or pharmaceutically acceptable salts thereof.

In one aspect of the third embodiment, the invention is directed to the use of compounds of Formula (III) to treat dyslipidemia in a mammalian subject wherein B¹ is Arg-Lys-Gln-Lys-(Arg)₅ (SEQ ID NO:8), Arg-(Lys)₂-Arg-Gln-(Arg)₄ (SEQ ID NO:9), Arg-(Lys)₂-(Arg)₃-Gln-(Arg)₂ (SEQ ID NO:10), Arg-(Lys)₂-(Arg)₄-Gln-Arg (SEQ ID NO:11), Arg-(Lys)₂-(Arg)₅-Gln (SEQ ID NO:12), Arg-(Lys)₂-Gln-(Arg)₅ (SEQ ID NO:13), Arg-Gln-(Lys)₂-(Arg)₅(SEQ ID NO:14), Arg-Gln-(Arg)₇ (SEQ ID NO:15), Arg-Gln-(Arg)s (SEQ ID NO:16), (Arg)₂-Gln-(Arg)₆(SEQ ID NO:17), (Arg)₂-Gln-(Arg)₇(SEQ ID NO:18), (Arg)₃-Gln-(Arg)₅(SEQ ID NO:19), (Arg)₃-Gln-(Arg)₆(SEQ ID NO:20), (Arg)₄-Gln-(Arg)₄(SEQ ID NO:21), (Arg)₄-Gln-(Arg)₅(SEQ ID NO:22), (Arg)₅(SEQ ID NO:23), (Arg)₅-Gln-(Arg)₃(SEQ ID NO:24), (Arg)₅-Gln-(Arg)₄(SEQ ID NO:25), (Arg)₆(SEQ ID NO:26), (Arg)₆-Gln-(Arg)₃(SEQ ID NO:27), (Arg)₇(SEQ ID NO:28), (Arg)₇-Gln-(Arg)₂(SEQ ID NO:29), (Arg)s (SEQ ID NO:30), (Arg)ₛ-Gln-Arg (SEQ ID NO:31), (Arg)₉(SEQ ID NO:32), (Arg)₉-Gln (SEQ ID NO:33), (D-Arg)₅, (D-Arg)₆, (D-Arg)₇, (D-Arg)ₛ, (D-Arg)₉, Gln-Arg-(Lys)₂-(Arg)₅(SEQ ID NO:34), Gln-(Arg)s (SEQ ID NO:35), Gln-(Arg)₉(SEQ ID NO:36), Tyr-Gly-Arg-(Lys)₂-(Arg)₂-Gln-(Arg)₃(SEQ ID NO:37), Tyr-Gly-Arg-(Lys)₂-(Arg)₂-Gln-(Arg)₃-Doc (SEQ ID NO:38); or deleted;

B² is β-Ala, β-Ala-Gly, β-Ala-Tyr, β-Ala-Tyr-Gly, (β-Ala)₂, (β-Ala)₂-Gly, (β-Ala)₂-Tyr, (β-Ala)₂-Tyr-Gly (SEQ ID NO:39), Doc, Doc-Gly, Doc-Tyr, Doc-Tyr-Gly, (Doc)₂, (Doc)₂-Gly, (Doc)₂-Tyr, (Doc)₂-Tyr-Gly (SEQ ID NO:40), or deleted;

B³ is Arg-Lys-Gln-Lys-(Arg)₅(SEQ ID NO:8), Arg-Lys-(Arg)₃-Gln-(Arg)₃(SEQ ID NO:41), Arg-(Lys)₂-Arg-Gln-(Arg)₄(SEQ ID NO:9), Arg-(Lys)₂-Gln-(Arg)₅(SEQ ID NO:13), Arg-(Lys)₂-(Arg)₂-Gln-(Arg)₃(SEQ ID NO:1), Arg-(Lys)₂-(Arg)₃-Gln-(Arg)₂ (SEQ ID NO:10), Arg-(Lys)₂-(Arg)₄-Gln-Arg (SEQ ID NO:11), Arg-(Lys)₂-(Arg)₅-Gln (SEQ ID NO:12), Arg-Gln-(Lys)₂-(Arg)₅(SEQ ID NO:14), Arg-Gln-(Arg)₇(SEQ ID NO:15), Arg-Gln-(Arg)s (SEQ ID NO:16), (Arg)₂-Lys-(Arg)₂-Gln-(Arg)₃(SEQ ID NO:42), (Arg)₂-Gln-(Arg)₆(SEQ ID NO:17), (Arg)₂-Gln-(Arg)₇(SEQ ID NO:18), (Arg)₃-Gln-(Arg)₅(SEQ ID NO:19), (Arg)₃-Gln-(Arg)₆(SEQ ID NO:20), (Arg)₄-Gln-(Arg)₄(SEQ ID NO:21), (Arg)₄-Gln-(Arg)₅(SEQ ID NO:22), (Arg)₅(SEQ ID NO:23), (Arg)₅-Gln-(Arg)₃(SEQ ID NO:24), (Arg)₅-Gln-(Arg)₄(SEQ ID NO:25), (Arg)₆(SEQ ID NO:26), (Arg)₆-Gln-(Arg)₃(SEQ ID NO:27), (Arg)₇(SEQ ID NO:28), (Arg)₇-Gln-(Arg)₂(SEQ ID NO:29), (Arg)s (SEQ ID NO:30), (Arg)ₛ-Gln-Arg (SEQ ID NO:31), (Arg)₉(SEQ ID NO:32), (Arg)₉-Gln (SEQ ID NO:33), (D-Arg)₅, (D-Arg)₆, (D-Arg)₇, (D-Arg)ₛ, (D-Arg)₉, Gln-Arg-(Lys)₂-(Arg)₅(SEQ ID NO:34), Gln-(Arg)s (SEQ ID NO:35), Gln-(Arg)₉(SEQ ID NO:36), or deleted;

A¹ is A6c, Cha, hCha, Chg, D-Chg, hChg, Gaba, hLeu, Met, β-hMet, D-2-Nal, Nip, Nle, Oic, Phe, D-Phe, hPhe, hPro, or deleted;

A² is Cys

A³ is D-Abu, Aib, Ala, β-Ala, D-Ala, D-Cha, Gaba, Glu, Gly, D-Ile, D-Leu, D-Met, D-Nle, D-Phe, D-Tle, D-Trp, D-Tyr, D-Val, or deleted;

A⁴ is H is;

A⁵ is D-Bal, D-1-Nal, D-2-Nal, D-Phe, D-(X¹,X²,X³,X⁴,X⁵)Phe, D-Trp, or D-(Et)Tyr;

A⁶ is Arg or hArg;

A⁷ is Bal, Bip, 1-Nal, 2-Nal, Trp, or D-Trp;

A⁸ is A5c, A6c, Aha, Ahx, Ala, β-Ala, Apn, Gaba, Gly, or deleted;

A⁹ is Cys, D-Cys, hCys, D-hCys, Lys, Pen, or D-Pen;

A¹⁰ is Pro, Thr or deleted;

A¹¹ is Pro or deleted;

A¹² is arg, Lys, or deleted;

A¹³ is Asp or deleted;

each of R² and R³ is, independently, H or acyl;

or pharmaceutically acceptable salts thereof.

Preferred ligands of the immeduiately foregoing group of compounds according to Formula (III), useful to treat dyslipidemia in a mammalian subject are compounds of the formula:

### Tyr-Gly-Arg-(Lys)₂-(Arg)₂-Gln-(Arg)₃-Nle-c(Asp-His-D-2-Nal-Arg-Trp-Lys)-NH₂;

### Tyr-Gly-Arg-(Lys)₂-(Arg)₂-Gln-(Arg)₃-Doc-Nle-c(Asp-His-D-2-Nal-Arg-Trp-Lys)-NH₂;

### Nle-c(Asp-His-D-2-Nal-Arg-Trp-Lys)-β-Ala-Tyr-Gly-Arg-(Lys)₂-(Arg)₂-Gln-(Arg)₃-NH₂;

### Ac-Nle-c(Asp-His-D-2-Nal-Arg-Trp-Lys)-β-Ala-Tyr-Gly-Arg-(Lys)₂-(Arg)₂-Gln-(Arg)₃-NH₂;

### Nle-c(Asp-His-D-2-Nal-Arg-Trp-Lys)-(Doc)₂-Tyr-Gly-Arg-(Lys)₂-(Arg)₂-Gln-(Arg)₃-NH₂;

### Ac-Nle-c(Asp-His-D-2-Nal-Arg-Trp-Lys)-(Pro)₂-Lys-Asp-Tyr-Gly-Arg-(Lys)₂-(Arg)₂-Gln-(Arg)₃-NH₂;

### Ac-c(Cys-Glu-His-D-2-Nal-Arg-Trp-Gly-Cys)-(Pro)₂-Lys-Asp-Tyr-Gly-Arg-(Lys)₂-(Arg)₂-Gln-(Arg)₃-NH₂;

### Ac-Nle-c(Asp-His-D-2-Nal-Arg-Trp-Lys)-(β-Ala)₂-Tyr-Gly-Arg-(Lys)₂-(Arg)₂-Gln-(Arg)₃-NH₂;

### Ac-Nle-c(Asp-His-D-2-Nal-Arg-Trp-Lys)-(Pro)₂-Lys-Asp-Doc-Tyr-Gly-Arg-(Lys)₂-(Arg)₂-Gln-(Arg)₃-NH₂;

### Ac-c(Cys-Glu-His-D-2-Nal-Arg-Trp-Gly-Cys)-(Pro)₂-Lys-Asp-Doc-Tyr-Gly-Arg-(Lys)₂-(Arg)₂-Gln-(Arg)₃-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₂-Gln-(Arg)₃-NH₂;

### Ac-c(Cys-Glu-His-D-2-Nal-Arg-Trp-Ala-Cys)-(Pro)₂-Lys-Asp-Doc-Tyr-Gly-Arg-(Lys)₂-(Arg)₂-Gln-(Arg)₃-NH₂;

### Ac-Nle-c(Asp-His-D-2-Nal-Arg-Trp-Lys)-(Doc)₂-Tyr-Gly-Arg-(Lys)₂-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-Arg-(Lys)₂-(Arg)₂-Gln-(Arg)₃-NH₂;

### Ac-c(Cys-Glu-His-D-2-Nal-Arg-Bal-Ala-Cys)-(Pro)₂-Lys-Asp-β-Ala-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-Gly-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-(Arg)₅-Gln-(Arg)₃-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-Arg-Gln-(Arg)₄-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-Gln-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-Lys-Gln-Lys-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₄-Gln-Arg-NH₂;

### Ac-c(Cys-Glu-His-D-2-Nal-Arg-Bal-Ala-Cys)-(Pro)₂-Lys-Asp-β-Ala-Tyr-Aib-Arg-(Lys)₂-(Arg)₂-Gln-(Arg)₃-NH₂;

### Ac-c(Cys-Glu-His-D-2-Nal-Arg-1-Nal-Ala-Cys)-(Pro)₂-Arg-Asp-β-Ala-(Arg)₅-Gln-(Arg)₃-NH₂;

### Ac-c(Cys-Glu-His-D-2-Nal-Arg-1-Nal-Ala-Cys)-(Pro)₂-Lys-Asp-β-Ala-(Arg)₅-Gln-(Arg)₃-NH₂;

### Ac-c(Cys-Glu-His-D-2-Nal-Arg-1-Nal-Ala-Cys)-(Pro)₂-Lys-Asp-β-Ala-(Arg)₆-Gln-(Arg)₃-NH₂;

### Ac-c(Cys-Glu-His-D-2-Nal-Arg-2-Nal-Ala-Cys)-(Pro)₂-Arg-Asp-β-Ala-(Arg)₅-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₅-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₆-Gln-(Arg)₃-NH₂;

### Ac-c(Cys-Glu-His-D-2-Nal-Arg-Bal-Ala-Cys)-(Pro)₂-Arg-Asp-β-Ala-(Arg)₆-Gln-(Arg)₃-NH₂;

### Ac-c(Cys-Glu-His-D-2-Nal-Arg-Bal-Ala-Cys)-(Pro)₂-Arg-Asp-β-Ala-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-(Arg)₆-Gln-(Arg)₃-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₃-Gln-(Arg)₂-NH₂;

### Ac-c(Cys-Glu-His-D-2-Nal-Arg-Trp-Ala-Cys)-(Pro)₂-Lys-Asp-β-Ala-Tyr-Gly-Arg-Gln-(Lys)₂-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₅-Gln-NH₂;

### Ac-c(Cys-Glu-His-D-2-Nal-Arg-1-Nal-Ala-Cys)-(Pro)₂-Lys-Asp-β-Ala-Tyr-Gly-Arg-(Lys)₂-(Arg)₂-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-Arg-(Lys)₂-(Arg)₂-Gln-(Arg)₃-NH₂;

### Ac-c(Cys-Glu-His-D-2-Nal-Arg-1-Nal-Ala-Cys)-(Pro)₂-Lys-Asp-β-Ala-(Arg)₂-Lys-(Arg)₂-Gln-(Arg)₃-NH₂;

### Ac-c(Cys-Glu-His-D-2-Nal-Arg-1-Nal-Ala-Cys)-(Pro)₂-Lys-Asp-β-Ala-Arg-Lys-(Arg)₃-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Gly-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Gly-Arg-Lys-(Arg)₃-Gln-(Arg)₃-NH₂;

### Ac-c(Cys-Glu-His-D-2-Nal-Arg-1-Nal-Ala-Cys)-(Pro)₂-Lys-Asp-β-Ala-(Arg)₂-Lys-(Arg)₂-Gln-(Arg)₃-NH₂;

### Ac-c(Cys-Glu-His-D-2-Nal-Arg-1-Nal-Ala-Cys)-(Pro)₂-Lys-Asp-β-Ala-Arg-Lys-(Arg)₃-Gln-(Arg)₃-NH₂;

### Ac-c(Cys-Glu-His-D-2-Nal-Arg-1-Nal-Ala-Cys)-(Pro)₂-Lys-Asp-β-Ala-Tyr-Gly-(Arg)₂-Lys-(Arg)₂-Gln-(Arg)₃-NH₂;

### Ac-c(Cys-Glu-His-D-2-Nal-Arg-1-Nal-Ala-Cys)-(Pro)₂-Lys-Asp-β-Ala-Tyr-Gly-Arg-Lys-(Arg)₃-Gln-(Arg)₃-NH₂;

### Ac-c(Cys-Glu-His-D-2-Nal-Arg-1-Nal-Ala-Cys)-(Pro)₂-Lys-Asp-β-Ala-Gly-(Arg)₂-Lys-(Arg)₂-Gln-(Arg)₃-NH₂;

### Ac-c(Cys-Glu-His-D-2-Nal-Arg-1-Nal-Ala-Cys)-(Pro)₂-Lys-Asp-β-Ala-Gly-Arg-Lys-(Arg)₃-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Arg-Lys-(Arg)₃-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-Arg-Lys-(Arg)₃-Gln-(Arg)₃-NH₂;

### Ac-c(Cys-Glu-His-D-2-Nal-Arg-2-Nal-Ala-Cys)-(Pro)₂-Lys-Asp-β-Ala-Gly-(Arg)₂-Lys-(Arg)₂-Gln-(Arg)₃-NH₂;

### Ac-c(Cys-Glu-His-D-2-Nal-Arg-2-Nal-Ala-Cys)-(Pro)₂-Lys-Asp-β-Ala-Gly-Arg-Lys-(Arg)₃-Gln-(Arg)₃-NH₂;

### Ac-c(Cys-Glu-His-D-2-Nal-Arg-Bal-Ala-Cys)-(Pro)₂-Lys-Asp-β-Ala-(Arg)₂-Lys-(Arg)₂-Gln-(Arg)₃-NH₂;

### Ac-c(Cys-Glu-His-D-2-Nal-Arg-Bal-Ala-Cys)-(Pro)₂-Lys-Asp-β-Ala-Arg-Lys-(Arg)₃-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-(Arg)₂-Lys-(Arg)₂-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-Arg-Lys-(Arg)₃-Gln-(Arg)₃-NH₂;

### Ac-c(Cys-Glu-His-D-2-Nal-Arg-Bal-Ala-Cys)-(Pro)₂-Lys-Asp-β-Ala-Gly-(Arg)₂-Lys-(Arg)₂-Gln-(Arg)₃-NH₂;

### Ac-c(Cys-Glu-His-D-2-Nal-Arg-Bal-Ala-Cys)-(Pro)₂-Lys-Asp-β-Ala-Gly-Arg-Lys-(Arg)₃-Gln-(Arg)₃-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-Arg-Gln-(Arg)₄-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-Gln-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-Lys-Gln-Lys-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-Gln-(Lys)₂-(Arg)₅-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₃-Gln-(Arg)₂-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₄-Gln-Arg-NH₂;

### Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₅-Gln-NH₂;

### Ac-c(Cys-Glu-His-D-2-Nal-Arg-Trp-Ala-Cys)-(Pro)₂-Lys-Asp-β-Ala-(Arg)₅-Gln-(Arg)₃-NH₂;

### Ac-c(Cys-Glu-His-D-2-Nal-Arg-Trp-Ala-Cys)-(Pro)₂-Arg-Asp-β-Ala-(Arg)₅-Gln-(Arg)₃-NH₂;

### Ac-c(Cys-Glu-His-D-2-Nal-Arg-Trp-Ala-Cys)-(Pro)₂-Lys-Asp-β-Ala-Tyr-Gly-(Arg)₅-Gln-(Arg)₃-NH₂;

### Ac-c(Cys-Glu-His-D-2-Nal-Arg-Trp-Ala-Cys)-(Pro)₂-Arg-Asp-β-Ala-Tyr-Gly-(Arg)₅-Gln-(Arg)₃-NH₂;

### Ac-c(Cys-Glu-His-D-2-Nal-Arg-Trp-Ala-Cys)-(Pro)₂-Lys-Asp-β-Ala-(Arg)₅-Gln-(Arg)₄-NH₂;

### Ac-c(Cys-Glu-His-D-2-Nal-Arg-Trp-Ala-Cys)-(Pro)₂-Arg-Asp-β-Ala-(Arg)₅-Gln-(Arg)₄-NH₂;

### Ac-c(Cys-Glu-His-D-2-Nal-Arg-Trp-Ala-Cys)-(Pro)₂-Lys-Asp-β-Ala-Tyr-Gly-(Arg)₅-Gln-(Arg)₄-NH₂;

### Ac-c(Cys-Glu-His-D-2-Nal-Arg-Trp-Ala-Cys)-(Pro)₂-Arg-Asp-β-Ala-Tyr-Gly-(Arg)₅-Gln-(Arg)₄-NH₂;

### Ac-c(Cys-Glu-His-D-2-Nal-Arg-1-Nal-Ala-Cys)-(Pro)₂-Lys-Asp-β-Ala-(Arg)₅-Gln-(Arg)₃-NH₂;

### Ac-c(Cys-Glu-His-D-2-Nal-Arg-1-Nal-Ala-Cys)-(Pro)₂-Arg-Asp-β-Ala-(Arg)₅-Gln-(Arg)₃-NH₂;

### Ac-c(Cys-Glu-His-D-2-Nal-Arg-1-Nal-Ala-Cys)-(Pro)₂-Lys-Asp-β-Ala-(Arg)₅-Gln-(Arg)₄-NH₂;

### Ac-c(Cys-Glu-His-D-2-Nal-Arg-1-Nal-Ala-Cys)-(Pro)₂-Arg-Asp-β-Ala-(Arg)₅-Gln-(Arg)₄-NH₂;

### Ac-c(Cys-Glu-His-D-2-Nal-Arg-1-Nal-Ala-Cys)-(Pro)₂-Lys-Asp-β-Ala-(Arg)₆-Gln-(Arg)₃-NH₂;

### Ac-c(Cys-Glu-His-D-2-Nal-Arg-1-Nal-Ala-Cys)-(Pro)₂-Arg-Asp-β-Ala-(Arg)₆-Gln-(Arg)₃-NH₂;

### Ac-c(Cys-Glu-His-D-2-Nal-Arg-1-Nal-Ala-Cys)-(Pro)₂-Lys-Asp-β-Ala-Tyr-Gly-(Arg)₅-Gln-(Arg)₃-NH₂;

### Ac-c(Cys-Glu-His-D-2-Nal-Arg-1-Nal-Ala-Cys)-(Pro)₂-Arg-Asp-β-Ala-Tyr-Gly-(Arg)₅-Gln-(Arg)₃-NH₂;

### Ac-c(Cys-Glu-His-D-2-Nal-Arg-1-Nal-Ala-Cys)-(Pro)₂-Lys-Asp-β-Ala-Tyr-Gly-(Arg)₅-Gln-(Arg)₄-NH₂;

### Ac-c(Cys-Glu-His-D-2-Nal-Arg-1-Nal-Ala-Cys)-(Pro)₂-Arg-Asp-β-Ala-Tyr-Gly-(Arg)₅-Gln-(Arg)₄-NH₂;

### Ac-c(Cys-Glu-His-D-2-Nal-Arg-1-Nal-Ala-Cys)-(Pro)₂-Lys-Asp-β-Ala-Tyr-Gly-(Arg)₆-Gln-(Arg)₃-NH₂;

### Ac-c(Cys-Glu-His-D-2-Nal-Arg-1-Nal-Ala-Cys)-(Pro)₂-Arg-Asp-β-Ala-Tyr-Gly-(Arg)₆-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₅-Gln-(Arg)₃-NH₂;

### Ac-c(Cys-Glu-His-D-2-Nal-Arg-2-Nal-Ala-Cys)-(Pro)₂-Arg-Asp-β-Ala-(Arg)₅-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₆-Gln-(Arg)₃-NH₂;

### Ac-c(Cys-Glu-His-D-2-Nal-Arg-2-Nal-Ala-Cys)-(Pro)₂-Arg-Asp-β-Ala-(Arg)₆-Gln-(Arg)₃-NH₂;

### Ac-c(Cys-Glu-His-D-2-Nal-Arg-2-Nal-Ala-Cys)-(Pro)₂-Lys-Asp-β-Ala-(Arg)₅-Gln-(Arg)₄-NH₂;

### Ac-c(Cys-Glu-His-D-2-Nal-Arg-2-Nal-Ala-Cys)-(Pro)₂-Arg-Asp-β-Ala-(Arg)₅-Gln-(Arg)₄-NH₂;

### Ac-c(Cys-Glu-His-D-2-Nal-Arg-2-Nal-Ala-Cys)-(Pro)₂-Lys-Asp-β-Ala-Tyr-Gly-(Arg)₅-Gln-(Arg)₃-NH₂;

### Ac-c(Cys-Glu-His-D-2-Nal-Arg-2-Nal-Ala-Cys)-(Pro)₂-Arg-Asp-β-Ala-Tyr-Gly-(Arg)₅-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-(Arg)₆-Gln-(Arg)₃-NH₂;

### Ac-c(Cys-Glu-His-D-2-Nal-Arg-2-Nal-Ala-Cys)-(Pro)₂-Arg-Asp-β-Ala-Tyr-Gly-(Arg)₆-Gln-(Arg)₃-NH₂;

### Ac-c(Cys-Glu-His-D-2-Nal-Arg-2-Nal-Ala-Cys)-(Pro)₂-Lys-Asp-β-Ala-Tyr-Gly-(Arg)₅-Gln-(Arg)₄-NH₂;

### Ac-c(Cys-Glu-His-D-2-Nal-Arg-2-Nal-Ala-Cys)-(Pro)₂-Arg-Asp-β-Ala-Tyr-Gly-(Arg)₅-Gln-(Arg)₄-NH₂;

### Ac-c(Cys-Glu-His-D-2-Nal-Arg-Bal-Ala-Cys)-(Pro)₂-Lys-Asp-β-Ala-(Arg)₅-Gln-(Arg)₃-NH₂;

### Ac-c(Cys-Glu-His-D-2-Nal-Arg-Bal-Ala-Cys)-(Pro)₂-Arg-Asp-β-Ala-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-(Arg)₅-Gln-(Arg)₄-NH₂;

### Ac-c(Cys-Glu-His-D-2-Nal-Arg-Bal-Ala-Cys)-(Pro)₂-Arg-Asp-β-Ala-(Arg)₅-Gln-(Arg)₄-NH₂;

### Ac-c(Cys-Glu-His-D-2-Nal-Arg-Bal-Ala-Cys)-(Pro)₂-Lys-Asp-β-Ala-(Arg)₆-Gln-(Arg)₃-NH₂;

### Ac-c(Cys-Glu-His-D-2-Nal-Arg-Bal-Ala-Cys)-(Pro)₂-Arg-Asp-β-Ala-(Arg)₆-Gln-(Arg)₃-NH₂;

### Ac-c(Cys-Glu-His-D-2-Nal-Arg-Bal-Ala-Cys)-(Pro)₂-Lys-Asp-β-Ala-Gly-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-(Arg)₅-Gln-(Arg)₃-NH₂;

### Ac-c(Cys-Glu-His-D-2-Nal-Arg-Bal-Ala-Cys)-(Pro)₂-Arg-Asp-β-Ala-Tyr-Gly-(Arg)₅-Gln-(Arg)₃-NH₂;

### Ac-c(Cys-Glu-His-D-2-Nal-Arg-Bal-Ala-Cys)-(Pro)₂-Lys-Asp-β-Ala-Tyr-Gly-(Arg)₅-Gln-(Arg)₄-NH₂;

### Ac-c(Cys-Glu-His-D-2-Nal-Arg-Bal-Ala-Cys)-(Pro)₂-Arg-Asp-β-Ala-Tyr-Gly-(Arg)₅-Gln-(Arg)₄-NH₂;

### Ac-c(Cys-Glu-His-D-2-Nal-Arg-Bal-Ala-Cys)-(Pro)₂-Lys-Asp-β-Ala-Tyr-Gly-(Arg)₆-Gln-(Arg)₃-NH₂;

### Ac-c(Cys-Glu-His-D-2-Nal-Arg-Bal-Ala-Cys)-(Pro)₂-Arg-Asp-β-Ala-Tyr-Gly-(Arg)₆-Gln-(Arg)₃-NH₂;

### Ac-Nle-c(Cys-D-Ala-His-D-Phe-Arg-Trp-Cys)-(Doc)₂-Tyr-Gly-Arg-(Lys)₂-(Arg)₂-Gln-(Arg)₃-NH₂;

### Ac-Nle-c(Cys-D-Ala-His-D-Phe-Arg-Trp-Cys)-β-Ala-Tyr-Gly-Arg-(Lys)₂-Arg-Gln-(Arg)₄-NH₂;

### Ac-Nle-c(Cys-D-Ala-His-D-Phe-Arg-Trp-Cys)-Doc-Tyr-Gly-Arg-(Lys)₂-(Arg)₂-Gln-(Arg)₃-NH₂;

### Ac-Nle-c(Cys-D-Ala-His-D-Phe-Arg-Trp-Cys)-β-Ala-(Arg)₅-Gln-(Arg)₃-NH₂;

### Ac-Nle-c(Cys-D-Ala-His-D-Phe-Arg-Trp-Cys)-β-Ala-Gly-(Arg)₅-Gln-(Arg)₃-NH₂;

### Ac-Nle-c(Cys-D-Ala-His-D-Phe-Arg-Trp-Cys)-β-Ala-Gly-(Arg)₅-Gln-(Arg)₃-NH₂;

### Ac-Nle-c(Cys-D-Ala-His-D-Phe-Arg-Trp-Cys)-β-Ala-Tyr-Gly-(Arg)₅-Gln-(Arg)₃-NH₂;

### Ac-Nle-c(Cys-D-Ala-His-D-Phe-Arg-Trp-Cys)-β-Ala-Gly-(Arg)₅-Gln-(Arg)₄-NH₂;

### Ac-Nle-c(Cys-D-Ala-His-D-Phe-Arg-Trp-Cys)-β-Ala-Tyr-Gly-(Arg)₂-Lys-(Arg)₂-Gln-(Arg)₃-NH₂;

### Ac-Nle-c(Cys-D-Ala-His-D-Phe-Arg-Trp-Cys)-β-Ala-Tyr-Gly-Arg-Lys-(Arg)₃-Gln-(Arg)₃-NH₂;

### Ac-Nle-c(Cys-D-Ala-His-D-Phe-Arg-Trp-Cys)-β-Ala-Gly-(Arg)₂-Lys-(Arg)₂-Gln-(Arg)₃-NH₂;

### Ac-Nle-c(Cys-D-Ala-His-D-Phe-Arg-Trp-Cys)-β-Ala-Gly-Arg-Lys-(Arg)₃-Gln-(Arg)₃-NH₂;

### Ac-Nle-c(Cys-D-Ala-His-D-Phe-Arg-Trp-Cys)-β-Ala-(Arg)₂-Lys-(Arg)₂-Gln-(Arg)₃-NH₂;

### Ac-Nle-c(Cys-D-Ala-His-D-Phe-Arg-Trp-Cys)-β-Ala-Arg-Lys-(Arg)₃-Gln-(Arg)₃-NH₂;

### Ac-Nle-c(Cys-D-Ala-His-D-Phe-Arg-Trp-Cys)-β-Ala-Tyr-Gly-Arg-(Lys)₂-Arg-Gln-(Arg)₄-NH₂;

### Ac-Nle-c(Cys-D-Ala-His-D-Phe-Arg-Trp-Cys)-Doc-Tyr-Gly-Arg-(Lys)₂-(Arg)₂-Gln-(Arg)₃-NH₂;

### Ac-Nle-c(Cys-D-Ala-His-D-Phe-Arg-Trp-Cys)-β-Ala-(Arg)₅-Gln-(Arg)₃-NH₂;

### Ac-Nle-c(Cys-D-Ala-His-D-Phe-Arg-Trp-Cys)-β-Ala-Gly-(Arg)₅-Gln-(Arg)₃-NH₂;

### Ac-Nle-c(Cys-D-Ala-His-D-Phe-Arg-Trp-Cys)-β-Ala-Tyr-Gly-(Arg)₅-Gln-(Arg)₃-NH₂;

### Ac-Nle-c(Cys-D-Ala-His-D-Phe-Arg-Trp-Cys)-(β-Ala)₂-(Arg)₅-Gln-(Arg)₃-NH₂;

### Ac-Nle-c(Cys-D-Ala-His-D-Phe-Arg-Trp-Cys)-(β-Ala)₂-Gly-(Arg)₅-Gln-(Arg)₃-NH₂;

### Ac-Nle-c(Cys-D-Ala-His-D-Phe-Arg-Trp-Cys)-(β-Ala)₂-Tyr-Gly-(Arg)₅-Gln-(Arg)₃-NH₂;

### Ac-Nle-c(Cys-D-Ala-His-D-Phe-Arg-Trp-Cys)-Doc-(Arg)₅-Gln-(Arg)₃-NH₂;

### Ac-Nle-c(Cys-D-Ala-His-D-Phe-Arg-Trp-Cys)-Doc-Gly-(Arg)₅-Gln-(Arg)₃-NH₂;

### Ac-Nle-c(Cys-D-Ala-His-D-Phe-Arg-Trp-Cys)-Doc-Tyr-Gly-(Arg)₅-Gln-(Arg)₃-NH₂;

### Ac-Nle-c(Cys-D-Ala-His-D-Phe-Arg-Trp-Cys)-(Doc)₂-(Arg)₅-Gln-(Arg)₃-NH₂;

### Ac-Nle-c(Cys-D-Ala-His-D-Phe-Arg-Trp-Cys)-(Doc)₂-Gly-(Arg)₅-Gln-(Arg)₃-NH₂;

### Ac-Nle-c(Cys-D-Ala-His-D-Phe-Arg-Trp-Cys)-(Doc)₂-Tyr-Gly-(Arg)₅-Gln-(Arg)₃-NH₂;

### Ac-Nle-c(Cys-D-Ala-His-D-Phe-Arg-Trp-Cys)-β-Ala-(Arg)₅-Gln-(Arg)₄-NH₂;

### Ac-Nle-c(Cys-D-Ala-His-D-Phe-Arg-Trp-Cys)-β-Ala-Gly-(Arg)₅-Gln-(Arg)₄-NH₂;

### Ac-Nle-c(Cys-D-Ala-His-D-Phe-Arg-Trp-Cys)-β-Ala-Tyr-Gly-(Arg)₅-Gln-(Arg)₄-NH₂;

### Ac-Nle-c(Cys-D-Ala-His-D-Phe-Arg-Trp-Cys)-(β-Ala)₂-(Arg)₅-Gln-(Arg)₄-NH₂;

### Ac-Nle-c(Cys-D-Ala-His-D-Phe-Arg-Trp-Cys)-(β-Ala)₂-Gly-(Arg)₅-Gln-(Arg)₄-NH₂;

### Ac-Nle-c(Cys-D-Ala-His-D-Phe-Arg-Trp-Cys)-(β-Ala)₂-Tyr-Gly-(Arg)₅-Gln-(Arg)₄-NH₂;

### Ac-Nle-c(Cys-D-Ala-His-D-Phe-Arg-Trp-Cys)-Doc-(Arg)₅-Gln-(Arg)₄-NH₂;

### Ac-Nle-c(Cys-D-Ala-His-D-Phe-Arg-Trp-Cys)-Doc-Gly-(Arg)₅-Gln-(Arg)₄-NH₂;

### Ac-Nle-c(Cys-D-Ala-His-D-Phe-Arg-Trp-Cys)-Doc-Tyr-Gly-(Arg)₅-Gln-(Arg)₄-NH₂;

### Ac-Nle-c(Cys-D-Ala-His-D-Phe-Arg-Trp-Cys)-(Doc)₂-(Arg)₅-Gln-(Arg)₄-NH₂;

### Ac-Nle-c(Cys-D-Ala-His-D-Phe-Arg-Trp-Cys)-(Doc)₂-Gly-(Arg)₅-Gln-(Arg)₄-NH₂;

### Ac-Nle-c(Cys-D-Ala-His-D-Phe-Arg-Trp-Cys)-(Doc)₂-Tyr-Gly-(Arg)₅-Gln-(Arg)₄-NH₂;

### Ac-Nle-c(Cys-His-D-Phe-Arg-Trp-Gaba-Cys)-β-Ala-Tyr-Gly-(Arg)₅-Gln-(Arg)₃-NH₂;

### Ac-Nle-c(Cys-His-D-Phe-Arg-Trp-Gaba-Cys)-β-Ala-(Arg)₅-Gln-(Arg)₃-NH₂;

### Ac-Nle-c(Asp-His-D-Phe-Arg-Trp-Ala-Lys)-β-Ala-Tyr-Gly-(Arg)₅-Gln-(Arg)₃-NH₂;

### Ac-Nle-c(Asp-His-D-Phe-Arg-Trp-Ala-Lys)-β-Ala-(Arg)₅-Gln-(Arg)₃-NH₂;

### Ac-Nle-c(Asp-His-D-Phe-Arg-Trp-Lys)-β-Ala-Tyr-Gly-(Arg)₅-Gln-(Arg)₃-NH₂;

### Ac-Nle-c(Asp-His-D-Phe-Arg-Trp-Lys)-β-Ala-Gly-(Arg)₅-Gln-(Arg)₃-NH₂;

### Ac-Nle-c(Asp-His-D-Phe-Arg-Trp-Lys)-β-Ala-(Arg)₅-Gln-(Arg)₃-NH₂;

### Ac-Nle-c(Asp-His-D-Phe-Arg-Trp-Lys)-(β-Ala)₂-Tyr-Gly-(Arg)₅-Gln-(Arg)₃-NH₂;

### Ac-Nle-c(Asp-His-D-Phe-Arg-Trp-Lys)-(β-Ala)₂-Gly-(Arg)₅-Gln-(Arg)₃-NH₂;

### Ac-Nle-c(Asp-His-D-Phe-Arg-Trp-Lys)-(β-Ala)₂-(Arg)₅-Gln-(Arg)₃-NH₂;

### Ac-Nle-c(Asp-His-D-Phe-Arg-Trp-Lys)-Doc-Tyr-Gly-(Arg)₅-Gln-(Arg)₃-NH₂;

### Ac-Nle-c(Asp-His-D-Phe-Arg-Trp-Lys)-Doc-Gly-(Arg)₅-Gln-(Arg)₃-NH₂;

### Ac-Nle-c(Asp-His-D-Phe-Arg-Trp-Lys)-Doc-(Arg)₅-Gln-(Arg)₃-NH₂;

### Ac-Nle-c(Asp-His-D-Phe-Arg-Trp-Lys)-(Doc)₂-Tyr-Gly-(Arg)₅-Gln-(Arg)₃-NH₂;

### Ac-Nle-c(Asp-His-D-Phe-Arg-Trp-Lys)-(Doc)₂-Gly-(Arg)₅-Gln-(Arg)₃-NH₂;

### Ac-Nle-c(Asp-His-D-Phe-Arg-Trp-Lys)-(Doc)₂-(Arg)₅-Gln-(Arg)₃-NH₂;

### Ac-Nle-c(Asp-His-D-Phe-Arg-Trp-Lys)-β-Ala-Tyr-Gly-(Arg)₅-Gln-(Arg)₄-NH₂;

### Ac-Nle-c(Asp-His-D-Phe-Arg-Trp-Lys)-β-Ala-Gly-(Arg)₅-Gln-(Arg)₄-NH₂;

### Ac-Nle-c(Asp-His-D-Phe-Arg-Trp-Lys)-β-Ala-(Arg)₅-Gln-(Arg)₄-NH₂;

### Ac-Nle-c(Asp-His-D-Phe-Arg-Trp-Lys)-(β-Ala)₂-Tyr-Gly-(Arg)₅-Gln-(Arg)₄-NH₂;

### Ac-Nle-c(Asp-His-D-Phe-Arg-Trp-Lys)-(β-Ala)₂-Gly-(Arg)₅-Gln-(Arg)₄-NH₂;

### Ac-Nle-c(Asp-His-D-Phe-Arg-Trp-Lys)-(β-Ala)₂-(Arg)₅-Gln-(Arg)₄-NH₂;

### Ac-Nle-c(Asp-His-D-Phe-Arg-Trp-Lys)-Doc-Tyr-Gly-(Arg)₅-Gln-(Arg)₄-NH₂;

### Ac-Nle-c(Asp-His-D-Phe-Arg-Trp-Lys)-Doc-Gly-(Arg)₅-Gln-(Arg)₄-NH₂;

### Ac-Nle-c(Asp-His-D-Phe-Arg-Trp-Lys)-Doc-(Arg)₅-Gln-(Arg)₄-NH₂;

### Ac-Nle-c(Asp-His-D-Phe-Arg-Trp-Lys)-(Doc)₂-Tyr-Gly-(Arg)₅-Gln-(Arg)₄-NH₂;

### Ac-Nle-c(Asp-His-D-Phe-Arg-Trp-Lys)-(Doc)₂-Gly-(Arg)₅-Gln-(Arg)₄-NH₂;

### Ac-Nle-c(Asp-His-D-Phe-Arg-Trp-Lys)-(Doc)₂-(Arg)₅-Gln-(Arg)₄-NH₂;

### Ac-Nle-c(Asp-His-D-Phe-Arg-Trp-β-Ala-Lys)-β-Ala-Tyr-Gly-(Arg)₅-Gln-(Arg)₃-NH₂;

### Ac-Nle-c(Asp-His-D-Phe-Arg-Trp-β-Ala-Lys)-β-Ala-(Arg)₅-Gln-(Arg)₃-NH₂;

### Ac-Nle-c(Cys-His-D-Phe-Arg-Trp-Ahx-Cys)-β-Ala-Tyr-Gly-(Arg)₅-Gln-(Arg)₃-NH₂;

### Ac-Nle-c(Cys-His-D-Phe-Arg-Trp-Ahx-Cys)-β-Ala-(Arg)₅-Gln-(Arg)₃-NH₂;

### D-Phe-c(Cys-His-D-Phe-Arg-Trp-β-Ala-D-Cys)-Thr-β-Ala-Tyr-Gly-(Arg)₅-Gln-(Arg)₃-NH₂;

### D-Phe-c(Cys-His-D-Phe-Arg-Trp-β-Ala-D-Cys)-Thr-β-Ala-(Arg)₅-Gln-(Arg)₃-NH₂;

### Ac-Nle-c(Cys-His-D-Phe-Arg-Trp-Apn-Cys)-β-Ala-Tyr-Gly-(Arg)₅-Gln-(Arg)₃-NH₂;

### Ac-Nle-c(Cys-His-D-Phe-Arg-Trp-Apn-Cys)-β-Ala-(Arg)₅-Gln-(Arg)₃-NH₂;

### Ac-Cha-c(Asp-His-D-Phe-Arg-Trp-Gaba-Lys)-β-Ala-Tyr-Gly-(Arg)₅-Gln-(Arg)₃-NH₂;

### Ac-Cha-c(Asp-His-D-Phe-Arg-Trp-Gaba-Lys)-β-Ala-(Arg)₅-Gln-(Arg)₃-NH₂;

### Ac-Nle-c(Asp-His-D-Phe-Arg-Trp-Gaba-Lys)-β-Ala-Tyr-Gly-(Arg)₅-Gln-(Arg)₃-NH₂;

### Ac-Nle-c(Asp-His-D-Phe-Arg-Trp-Gaba-Lys)-β-Ala-(Arg)₅-Gln-(Arg)₃-NH₂;

### Ac-Chg-c(Asp-His-D-Phe-Arg-Trp-Gaba-Lys)-β-Ala-Tyr-Gly-(Arg)₅-Gln-(Arg)₃-NH₂;

### Ac-Chg-c(Asp-His-D-Phe-Arg-Trp-Gaba-Lys)-β-Ala-(Arg)₅-Gln-(Arg)₃-NH₂;

### Ac-hCha-c(Asp-His-D-Phe-Arg-Trp-Gaba-Lys)-β-Ala-Tyr-Gly-(Arg)₅-Gln-(Arg)₃-NH₂;

### Ac-hCha-c(Asp-His-D-Phe-Arg-Trp-Gaba-Lys)-β-Ala-(Arg)₅-Gln-(Arg)₃-NH₂;

### Ac-hCha-c(Asp-His-D-Phe-Arg-Trp-Gaba-Lys)-(β-Ala)₂-Tyr-Gly-(Arg)₅-Gln-(Arg)₃-NH₂;

### Ac-hCha-c(Asp-His-D-Phe-Arg-Trp-Gaba-Lys)-(β-Ala)₂-(Arg)₅-Gln-(Arg)₃-NH₂;

### Ac-hCha-c(Asp-His-D-Phe-Arg-Trp-Gaba-Lys)-Doc-Tyr-Gly-(Arg)₅-Gln-(Arg)₃-NH₂;

### Ac-hCha-c(Asp-His-D-Phe-Arg-Trp-Gaba-Lys)-Doc-(Arg)₅-Gln-(Arg)₃-NH₂;

### Ac-hCha-c(Asp-His-D-Phe-Arg-Trp-Gaba-Lys)-(Doc)₂-Tyr-Gly-(Arg)₅-Gln-(Arg)₃-NH₂;

### Ac-hCha-c(Asp-His-D-Phe-Arg-Trp-Gaba-Lys)-(Doc)₂-(Arg)₅-Gln-(Arg)₃-NH₂;

### Ac-hCha-c(Asp-His-D-Phe-Arg-Trp-Gaba-Lys)-β-Ala-Tyr-Gly-(Arg)₅-Gln-(Arg)₄-NH₂;

### Ac-hCha-c(Asp-His-D-Phe-Arg-Trp-Gaba-Lys)-β-Ala-(Arg)₅-Gln-(Arg)₄-NH₂;

### Ac-hCha-c(Asp-His-D-Phe-Arg-Trp-Gaba-Lys)-(β-Ala)₂-Tyr-Gly-(Arg)₅-Gln-(Arg)₄-NH₂;

### Ac-hCha-c(Asp-His-D-Phe-Arg-Trp-Gaba-Lys)-(β-Ala)₂-(Arg)₅-Gln-(Arg)₄-NH₂;

### Ac-hCha-c(Asp-His-D-Phe-Arg-Trp-Gaba-Lys)-Doc-Tyr-Gly-(Arg)₅-Gln-(Arg)₄-NH₂;

### Ac-hCha-c(Asp-His-D-Phe-Arg-Trp-Gaba-Lys)-Doc-(Arg)₅-Gln-(Arg)₄-NH₂;

### Ac-hCha-c(Asp-His-D-Phe-Arg-Trp-Gaba-Lys)-(Doc)₂-Tyr-Gly-(Arg)₅-Gln-(Arg)₄-NH₂;

### Ac-hCha-c(Asp-His-D-Phe-Arg-Trp-Gaba-Lys)-(Doc)₂-(Arg)₅-Gln-(Arg)₄-NH₂;

Ac-D-Chg-c(Asp-His-D-Phe-Arg-Trp-Gab a-Lys)-β-Ala-Tyr-Gly-(Arg)₅-Gln-(Arg)₃-NH₂;

### Ac-D-Chg-c(Asp-His-D-Phe-Arg-Trp-Gaba-Lys)-β-Ala-(Arg)₅-Gln-(Arg)₃-NH₂;

### Ac-hPhe-c(Asp-His-D-Phe-Arg-Trp-Gaba-Lys)-β-Ala-Tyr-Gly-(Arg)₅-Gln-(Arg)₃-NH₂;

### Ac-hPhe-c(Asp-His-D-Phe-Arg-Trp-Gaba-Lys)-β-Ala-(Arg)₅-Gln-(Arg)₃-NH₂;

### Ac-Nle-c(Cys-His-D-Phe-Arg-D-Trp-Apn-Cys)-β-Ala-Tyr-Gly-(Arg)₅-Gln-(Arg)₃-NH₂;

### Ac-Nle-c(Cys-His-D-Phe-Arg-D-Trp-Apn-Cys)-β-Ala-(Arg)₅-Gln-(Arg)₃-NH₂;

### Ac-Nle-c(Cys-His-D-Phe-Arg-D-Trp-Ahx-Cys)-β-Ala-Tyr-Gly-(Arg)₅-Gln-(Arg)₃-NH₂;

### Ac-Nle-c(Cys-His-D-Phe-Arg-D-Trp-Ahx-Cys)-β-Ala-(Arg)₅-Gln-(Arg)₃-NH₂;

### Ac-Nle-c(Cys-His-D-Phe-Arg-D-Trp-β-Ala-Cys)-β-Ala-Tyr-Gly-(Arg)₅-Gln-(Arg)₃-NH₂;

### Ac-Nle-c(Cys-His-D-Phe-Arg-D-Trp-β-Ala-Cys)-β-Ala-(Arg)₅-Gln-(Arg)₃-NH₂;

### Ac-Nle-c(Cys-D-Ala-His-D-Phe-Arg-Trp-Pen)-β-Ala-Tyr-Gly-(Arg)₅-Gln-(Arg)₃-NH₂;

### Ac-Nle-c(Cys-D-Ala-His-D-Phe-Arg-Trp-Pen)-β-Ala-Gly-(Arg)₅-Gln-(Arg)₃-NH₂;

### Ac-Nle-c(Cys-D-Ala-His-D-Phe-Arg-Trp-Pen)-β-Ala-(Arg)₅-Gln-(Arg)₃-NH₂;

### Ac-Nle-c(Cys-D-Ala-His-D-Phe-Arg-Trp-Pen)-(β-Ala)₂-Tyr-Gly-(Arg)₅-Gln-(Arg)₃-NH₂;

### Ac-Nle-c(Cys-D-Ala-His-D-Phe-Arg-Trp-Pen)-(β-Ala)₂-Gly-(Arg)₅-Gln-(Arg)₃-NH₂;

### Ac-Nle-c(Cys-D-Ala-His-D-Phe-Arg-Trp-Pen)-(β-Ala)₂-(Arg)₅-Gln-(Arg)₃-NH₂;

### Ac-Nle-c(Cys-D-Ala-His-D-Phe-Arg-Trp-Pen)-Doc-Tyr-Gly-(Arg)₅-Gln-(Arg)₃-NH₂;

### Ac-Nle-c(Cys-D-Ala-His-D-Phe-Arg-Trp-Pen)-Doc-Gly-(Arg)₅-Gln-(Arg)₃-NH₂;

### Ac-Nle-c(Cys-D-Ala-His-D-Phe-Arg-Trp-Pen)-Doc-(Arg)₅-Gln-(Arg)₃-NH₂;

### Ac-Nle-c(Cys-D-Ala-His-D-Phe-Arg-Trp-Pen)-(Doc)₂-Tyr-Gly-(Arg)₅-Gln-(Arg)₃-NH₂;

### Ac-Nle-c(Cys-D-Ala-His-D-Phe-Arg-Trp-Pen)-(Doc)₂-Gly-(Arg)₅-Gln-(Arg)₃-NH₂;

### Ac-Nle-c(Cys-D-Ala-His-D-Phe-Arg-Trp-Pen)-(Doc)₂-(Arg)₅-Gln-(Arg)₃-NH₂;

### D-Phe-c(Cys-His-D-(Et)Tyr-Arg-Trp-β-Ala-D-Cys)-β-Ala-Tyr-Gly-(Arg)₅-Gln-(Arg)₃-NH₂;

### D-Phe-c(Cys-His-D-(Et)Tyr-Arg-Trp-β-Ala-D-Cys)-β-Ala-(Arg)₅-Gln-(Arg)₃-NH₂;

### D-Phe-c(Cys-His-D-(Et)Tyr-Arg-Trp-β-Ala-D-Cys)-β-Ala-Gly-(Arg)₅-Gln-(Arg)₃-NH₂;

### D-Phe-c(Cys-His-D-(Et)Tyr-Arg-Trp-β-Ala-D-Cys)-β-Ala-(Arg)₅-Gln-(Arg)₄-NH₂;

### D-Phe-c(Cys-His-D-(Et)Tyr-Arg-Trp-β-Ala-D-Cys)-(β-Ala)₂-Tyr-Gly-(Arg)₅-Gln-(Arg)₃-NH₂;

### D-Phe-c(Cys-His-D-(Et)Tyr-Arg-Trp-β-Ala-D-Cys)-(β-Ala)₂-(Arg)₅-Gln-(Arg)₃-NH₂;

### D-Phe-c(Cys-His-D-(Et)Tyr-Arg-Trp-β-Ala-D-Cys)-(β-Ala)₂-Gly-(Arg)₅-Gln-(Arg)₃-NH₂;

### D-Phe-c(Cys-His-D-(Et)Tyr-Arg-Trp-β-Ala-D-Cys)-(β-Ala)₂-(Arg)₅-Gln-(Arg)₄-NH₂;

### D-Phe-c(Cys-His-D-(Et)Tyr-Arg-Trp-β-Ala-D-Cys)-Doc-Tyr-Gly-(Arg)₅-Gln-(Arg)₃-NH₂;

### D-Phe-c(Cys-His-D-(Et)Tyr-Arg-Trp-β-Ala-D-Cys)-Doc-(Arg)₅-Gln-(Arg)₃-NH₂;

### D-Phe-c(Cys-His-D-(Et)Tyr-Arg-Trp-β-Ala-D-Cys)-Doc-Gly-(Arg)₅-Gln-(Arg)₃-NH₂;

### D-Phe-c(Cys-His-D-(Et)Tyr-Arg-Trp-β-Ala-D-Cys)-Doc-(Arg)₅-Gln-(Arg)₄-NH₂;

### D-Phe-c(Cys-His-D-(Et)Tyr-Arg-Trp-β-Ala-D-Cys)-(Doc)₂-Tyr-Gly-(Arg)₅-Gln-(Arg)₃-NH₂;

### D-Phe-c(Cys-His-D-(Et)Tyr-Arg-Trp-β-Ala-D-Cys)-(Doc)₂-(Arg)₅-Gln-(Arg)₃-NH₂;

### D-Phe-c(Cys-His-D-(Et)Tyr-Arg-Trp-β-Ala-D-Cys)-(Doc)₂-Gly-(Arg)₅-Gln-(Arg)₃-NH₂;

### D-Phe-c(Cys-His-D-(Et)Tyr-Arg-Trp-β-Ala-D-Cys)-(Doc)₂-(Arg)₅-Gln-(Arg)₄-NH₂;

### D-Phe-c(Cys-His-D-(Et)Tyr-hArg-Trp-β-Ala-D-Cys)-Thr-β-Ala-Tyr-Gly-(Arg)₅-Gln-(Arg)₃-NH₂;

### D-Phe-c(Cys-His-D-(Et)Tyr-hArg-Trp-β-Ala-D-Cys)-Thr-β-Ala-(Arg)₅-Gln-(Arg)₃-NH₂;

### D-Phe-c(Cys-His-D-(Et)Tyr-hArg-Trp-β-Ala-D-Cys)-Thr-(β-Ala)₂-Tyr-Gly-(Arg)₅-Gln-(Arg)₃-NH₂;

### D-Phe-c(Cys-His-D-(Et)Tyr-hArg-Trp-β-Ala-D-Cys)-Thr-(β-Ala)₂-(Arg)₅-Gln-(Arg)₃-NH₂;

### D-Phe-c(Cys-His-D-(Et)Tyr-hArg-Trp-β-Ala-D-Cys)-Thr-Doc-Tyr-Gly-(Arg)₅-Gln-(Arg)₃-NH₂;

### D-Phe-c(Cys-His-D-(Et)Tyr-hArg-Trp-β-Ala-D-Cys)-Thr-Doc-(Arg)₅-Gln-(Arg)₃-NH₂;

### D-Phe-c(Cys-His-D-(Et)Tyr-hArg-Trp-β-Ala-D-Cys)-Thr-(Doc)₂-Tyr-Gly-(Arg)₅-Gln-(Arg)₃-NH₂;

### D-Phe-c(Cys-His-D-(Et)Tyr-hArg-Trp-β-Ala-D-Cys)-Thr-β-Ala-Tyr-Gly-(Arg)₅-Gln-(Arg)₄-NH₂;

### D-Phe-c(Cys-His-D-(Et)Tyr-hArg-Trp-β-Ala-D-Cys)-Thr-β-Ala-(Arg)₅-Gln-(Arg)₄-NH₂;

### D-Phe-c(Cys-His-D-(Et)Tyr-hArg-Trp-β-Ala-D-Cys)-Thr-(β-Ala)₂-Tyr-Gly-(Arg)₅-Gln-(Arg)₄-NH₂;

### D-Phe-c(Cys-His-D-(Et)Tyr-hArg-Trp-β-Ala-D-Cys)-Thr-(β-Ala)₂-(Arg)₅-Gln-(Arg)₄-NH₂;

### D-Phe-c(Cys-His-D-(Et)Tyr-hArg-Trp-β-Ala-D-Cys)-Thr-Doc-Tyr-Gly-(Arg)₅-Gln-(Arg)₄-NH₂;

### D-Phe-c(Cys-His-D-(Et)Tyr-hArg-Trp-β-Ala-D-Cys)-Thr-Doc-(Arg)₅-Gln-(Arg)₄-NH₂;

### D-Phe-c(Cys-His-D-(Et)Tyr-hArg-Trp-β-Ala-D-Cys)-Thr-(Doc)₂-Tyr-Gly-(Arg)₅-Gln-(Arg)₄-NH₂;

### D-Phe-c(Cys-His-D-(Et)Tyr-hArg-Trp-β-Ala-D-Cys)-Thr-(Doc)₂-(Arg)₅-Gln-(Arg)₄-NH₂;

### D-Phe-c(Cys-His-D-(Et)Tyr-hArg-Bip-β-Ala-D-Cys)-Thr-β-Ala-Tyr-Gly-(Arg)₅-Gln-(Arg)₃-NH₂;

### D-Phe-c(Cys-His-D-(Et)Tyr-hArg-Bip-β-Ala-D-Cys)-Thr-β-Ala-Tyr-Gly-(Arg)₅-Gln-(Arg)₄-NH₂;

### D-Phe-c(Cys-His-D-(Et)Tyr-hArg-Bip-β-Ala-D-Cys)-Thr-β-Ala-(Arg)₅-Gln-(Arg)₃-NH₂;

### D-Phe-c(Cys-His-D-(Et)Tyr-hArg-Bip-β-Ala-D-Cys)-Thr-(β-Ala)₂-Tyr-Gly-(Arg)₅-Gln-(Arg)₃-NH₂;

### D-Phe-c(Cys-His-D-(Et)Tyr-hArg-Bip-β-Ala-D-Cys)-Thr-(β-Ala)₂-(Arg)₅-Gln-(Arg)₃-NH₂;

### D-Phe-c(Cys-His-D-(Et)Tyr-hArg-Bip-β-Ala-D-Cys)-Thr-Doc-Tyr-Gly-(Arg)₅-Gln-(Arg)₃-NH₂;

### D-Phe-c(Cys-His-D-(Et)Tyr-hArg-Bip-β-Ala-D-Cys)-Thr-Doc-Tyr-Gly-(Arg)₅-Gln-(Arg)₄-NH₂;

### D-Phe-c(Cys-His-D-(Et)Tyr-hArg-Bip-β-Ala-D-Cys)-Thr-Doc-(Arg)₅-Gln-(Arg)₃-NH₂;

### D-Phe-c(Cys-His-D-(Et)Tyr-hArg-Bip-β-Ala-D-Cys)-Thr-(Doc)₂-Tyr-Gly-(Arg)₅-Gln-(Arg)₃-NH₂;

### D-Phe-c(Cys-His-D-(Et)Tyr-hArg-Bip-β-Ala-D-Cys)-Thr-(Doc)₂-(Arg)₅-Gln-(Arg)₃-NH₂;

### Ac-Nle-c(Cys-D-Ala-His-D-Phe-Arg-Trp-Gly-Cys)-β-Ala-Tyr-Gly-(Arg)₅-Gln-(Arg)₃-NH₂;

### Ac-Nle-c(Cys-D-Ala-His-D-Phe-Arg-Trp-Gly-Cys)-β-Ala-(Arg)₅-Gln-(Arg)₃-NH₂;

### Nle-c(Cys-His-D-Phe-Arg-Trp-Apn-Cys)-β-Ala-Tyr-Gly-(Arg)₅-Gln-(Arg)₃-NH₂;

### Nle-c(Cys-His-D-Phe-Arg-Trp-Apn-Cys)-β-Ala-(Arg)₅-Gln-(Arg)₃-NH₂;

### Nle-c(Cys-His-D-Phe-Arg-Trp-Apn-Cys)-(β-Ala)₂-Tyr-Gly-(Arg)₅-Gln-(Arg)₃-NH₂;

### Nle-c(Cys-His-D-Phe-Arg-Trp-Apn-Cys)-(β-Ala)₂-(Arg)₅-Gln-(Arg)₃-NH₂;

### Nle-c(Cys-His-D-Phe-Arg-Trp-Apn-Cys)-β-Ala-Tyr-Gly-(Arg)₅-Gln-(Arg)₄-NH₂;

### Nle-c(Cys-His-D-Phe-Arg-Trp-Apn-Cys)-β-Ala-(Arg)₅-Gln-(Arg)₄-NH₂;

### Nle-c(Cys-His-D-Phe-Arg-Trp-Apn-Cys)-(β-Ala)₂-Tyr-Gly-(Arg)₅-Gln-(Arg)₄-NH₂;

### Nle-c(Cys-His-D-Phe-Arg-Trp-Apn-Cys)-(β-Ala)₂-(Arg)₅-Gln-(Arg)₄-NH₂;

### Nle-c(Cys-His-D-Phe-Arg-Trp-Apn-Cys)-Doc-Tyr-Gly-(Arg)₅-Gln-(Arg)₃-NH₂;

### Nle-c(Cys-His-D-Phe-Arg-Trp-Apn-Cys)-Doc-(Arg)₅-Gln-(Arg)₃-NH₂;

### Nle-c(Cys-His-D-Phe-Arg-Trp-Apn-Cys)-(Doc)₂-Tyr-Gly-(Arg)₅-Gln-(Arg)₃-NH₂;

### Nle-c(Cys-His-D-Phe-Arg-Trp-Apn-Cys)-(Doc)₂-(Arg)₅-Gln-(Arg)₃-NH₂;

### Nle-c(Cys-His-D-Phe-Arg-Trp-Apn-Cys)-Doc-Tyr-Gly-(Arg)₅-Gln-(Arg)₄-NH₂;

### Nle-c(Cys-His-D-Phe-Arg-Trp-Apn-Cys)-Doc-(Arg)₅-Gln-(Arg)₄-NH₂;

### Nle-c(Cys-His-D-Phe-Arg-Trp-Apn-Cys)-(Doc)₂-Tyr-Gly-(Arg)₅-Gln-(Arg)₄-NH₂;

### Nle-c(Cys-His-D-Phe-Arg-Trp-Apn-Cys)-(Doc)₂-(Arg)₅-Gln-(Arg)₄-NH₂;

### Ac-Nle-c(Cys-D-Leu-His-D-Phe-Arg-Trp-Cys)-β-Ala-Tyr-Gly-(Arg)₅-Gln-(Arg)₃-NH₂;

### Ac-Nle-c(Cys-D-Leu-His-D-Phe-Arg-Trp-Cys)-β-Ala-(Arg)₅-Gln-(Arg)₃-NH₂;

### Ac-Nle-c(Cys-D-Leu-His-D-Phe-Arg-Trp-Cys)-(β-Ala)₂-Tyr-Gly-(Arg)₅-Gln-(Arg)₃-NH₂;

### Ac-Nle-c(Cys-D-Leu-His-D-Phe-Arg-Trp-Cys)-(β-Ala)₂-(Arg)₅-Gln-(Arg)₃-NH₂;

### Ac-Nle-c(Cys-D-Leu-His-D-Phe-Arg-Trp-Cys)-Doc-Tyr-Gly-(Arg)₅-Gln-(Arg)₃-NH₂;

### Ac-Nle-c(Cys-D-Leu-His-D-Phe-Arg-Trp-Cys)-Doc-(Arg)₅-Gln-(Arg)₃-NH₂;

### Ac-Nle-c(Cys-D-Leu-His-D-Phe-Arg-Trp-Cys)-(Doc)₂-Tyr-Gly-(Arg)₅-Gln-(Arg)₃-NH₂;

### Ac-Nle-c(Cys-D-Leu-His-D-Phe-Arg-Trp-Cys)-(Doc)₂-(Arg)₅-Gln-(Arg)₃-NH₂;

### Ac-Nle-c(Cys-D-Leu-His-D-Phe-Arg-Trp-Cys)-β-Ala-Tyr-Gly-(Arg)₅-Gln-(Arg)₄-NH₂;

### Ac-Nle-c(Cys-D-Leu-His-D-Phe-Arg-Trp-Cys)-β-Ala-(Arg)₅-Gln-(Arg)₄-NH₂;

### Ac-Nle-c(Cys-D-Leu-His-D-Phe-Arg-Trp-Cys)-(β-Ala)₂-Tyr-Gly-(Arg)₅-Gln-(Arg)₄-NH₂;

### Ac-Nle-c(Cys-D-Leu-His-D-Phe-Arg-Trp-Cys)-(β-Ala)₂-(Arg)₅-Gln-(Arg)₄-NH₂;

### Ac-Nle-c(Cys-D-Leu-His-D-Phe-Arg-Trp-Cys)-Doc-Tyr-Gly-(Arg)₅-Gln-(Arg)₄-NH₂;

### Ac-Nle-c(Cys-D-Leu-His-D-Phe-Arg-Trp-Cys)-Doc-(Arg)₅-Gln-(Arg)₄-NH₂;

### Ac-Nle-c(Cys-D-Leu-His-D-Phe-Arg-Trp-Cys)-(Doc)₂-Tyr-Gly-(Arg)₅-Gln-(Arg)₄-NH₂;

### Ac-Nle-c(Cys-D-Leu-His-D-Phe-Arg-Trp-Cys)-(Doc)₂-(Arg)₅-Gln-(Arg)₄-NH₂;

### Ac-Nle-c(Cys-D-Cha-His-D-Phe-Arg-Trp-Cys)-β-Ala-Tyr-Gly-(Arg)₅-Gln-(Arg)₃-NH₂;

### Ac-Nle-c(Cys-D-Cha-His-D-Phe-Arg-Trp-Cys)-β-Ala-(Arg)₅-Gln-(Arg)₃-NH₂;

### Ac-Nle-c(Cys-D-Cha-His-D-Phe-Arg-Trp-Cys)-(β-Ala)₂-Tyr-Gly-(Arg)₅-Gln-(Arg)₃-NH₂;

### Ac-Nle-c(Cys-D-Cha-His-D-Phe-Arg-Trp-Cys)-(β-Ala)₂-(Arg)₅-Gln-(Arg)₃-NH₂;

### Ac-Nle-c(Cys-D-Cha-His-D-Phe-Arg-Trp-Cys)-Doc-Tyr-Gly-(Arg)₅-Gln-(Arg)₃-NH₂;

### Ac-Nle-c(Cys-D-Cha-His-D-Phe-Arg-Trp-Cys)-Doc-(Arg)₅-Gln-(Arg)₃-NH₂;

### Ac-Nle-c(Cys-D-Cha-His-D-Phe-Arg-Trp-Cys)-(Doc)₂-Tyr-Gly-(Arg)₅-Gln-(Arg)₃-NH₂;

### Ac-Nle-c(Cys-D-Cha-His-D-Phe-Arg-Trp-Cys)-(Doc)₂-(Arg)₅-Gln-(Arg)₃-NH₂;

### Ac-Nle-c(Cys-D-Cha-His-D-Phe-Arg-Trp-Cys)-β-Ala-Tyr-Gly-(Arg)₅-Gln-(Arg)₄-NH₂;

### Ac-Nle-c(Cys-D-Cha-His-D-Phe-Arg-Trp-Cys)-β-Ala-(Arg)₅-Gln-(Arg)₄-NH₂;

### Ac-Nle-c(Cys-D-Cha-His-D-Phe-Arg-Trp-Cys)-(β-Ala)₂-Tyr-Gly-(Arg)₅-Gln-(Arg)₄-NH₂;

### Ac-Nle-c(Cys-D-Cha-His-D-Phe-Arg-Trp-Cys)-(β-Ala)₂-(Arg)₅-Gln-(Arg)₄-NH₂;

### Ac-Nle-c(Cys-D-Cha-His-D-Phe-Arg-Trp-Cys)-Doc-Tyr-Gly-(Arg)₅-Gln-(Arg)₄-NH₂;

### Ac-Nle-c(Cys-D-Cha-His-D-Phe-Arg-Trp-Cys)-Doc-(Arg)₅-Gln-(Arg)₄-NH₂;

### Ac-Nle-c(Cys-D-Cha-His-D-Phe-Arg-Trp-Cys)-(Doc)₂-Tyr-Gly-(Arg)₅-Gln-(Arg)₄-NH₂;

### Ac-Nle-c(Cys-D-Cha-His-D-Phe-Arg-Trp-Cys)-(Doc)₂-(Arg)₅-Gln-(Arg)₄-NH₂;

### Nle-c(Cys-His-D-Phe-Arg-Trp-Gaba-Cys)-β-Ala-Tyr-Gly-(Arg)₅-Gln-(Arg)₃-NH₂;

### Nle-c(Cys-His-D-Phe-Arg-Trp-Gaba-Cys)-β-Ala-(Arg)₅-Gln-(Arg)₃-NH₂;

### Nle-c(Cys-His-D-Phe-Arg-Trp-Gaba-Cys)-(β-Ala)₂-Tyr-Gly-(Arg)₅-Gln-(Arg)₃-NH₂;

### Nle-c(Cys-His-D-Phe-Arg-Trp-Gaba-Cys)-(β-Ala)₂-(Arg)₅-Gln-(Arg)₃-NH₂;

### Nle-c(Cys-His-D-Phe-Arg-Trp-Gaba-Cys)-β-Ala-Tyr-Gly-(Arg)₅-Gln-(Arg)₄-NH₂;

### Nle-c(Cys-His-D-Phe-Arg-Trp-Gaba-Cys)-β-Ala-(Arg)₅-Gln-(Arg)₄-NH₂;

### Nle-c(Cys-His-D-Phe-Arg-Trp-Gaba-Cys)-(β-Ala)₂-Tyr-Gly-(Arg)₅-Gln-(Arg)₄-NH₂;

### Nle-c(Cys-His-D-Phe-Arg-Trp-Gaba-Cys)-(β-Ala)₂-(Arg)₅-Gln-(Arg)₄-NH₂;

### Nle-c(Cys-His-D-Phe-Arg-Trp-Gaba-Cys)-Doc-Tyr-Gly-(Arg)₅-Gln-(Arg)₃-NH₂;

### Nle-c(Cys-His-D-Phe-Arg-Trp-Gaba-Cys)-Doc-(Arg)₅-Gln-(Arg)₃-NH₂;

### Nle-c(Cys-His-D-Phe-Arg-Trp-Gaba-Cys)-(Doc)₂-Tyr-Gly-(Arg)₅-Gln-(Arg)₃-NH₂;

### Nle-c(Cys-His-D-Phe-Arg-Trp-Gaba-Cys)-(Doc)₂-(Arg)₅-Gln-(Arg)₃-NH₂;

### Nle-c(Cys-His-D-Phe-Arg-Trp-Gaba-Cys)-Doc-Tyr-Gly-(Arg)₅-Gln-(Arg)₄-NH₂;

### Nle-c(Cys-His-D-Phe-Arg-Trp-Gaba-Cys)-Doc-(Arg)₅-Gln-(Arg)₄-NH₂;

### Nle-c(Cys-His-D-Phe-Arg-Trp-Gaba-Cys)-(Doc)₂-Tyr-Gly-(Arg)₅-Gln-(Arg)₄-NH₂; or

### Nle-c(Cys-His-D-Phe-Arg-Trp-Gaba-Cys)-(Doc)₂-(Arg)₅-Gln-(Arg)₄-NH₂;

or pharmaceutically acceptable salts thereof.

In a fourth embodiment, the invention provides a method to treat dyslipidemia in a mammalian subject by the administration of a therapeutically effective amount of a melanocortin receptor compound according to Formula (IV), and pharmaceutically acceptable salts, hydrates, solvates and prodrugs thereof, with a compound having the following formula (formula (IV)):

Ac-c(Cys-Glu-His-A¹-Arg-A²-A³-Cys)-(Pro)₂-Lys-Asp-NH₂ (IV)

wherein:
A¹ is the D-isomer of X-Phe or 2-Nal where X is halogen;

### A² is Bal, 1-Nal, 2-Nal, or Trp; and

### A³ is Aib, Ala, β-Ala or Gly,

or pharmaceutically acceptable salts thereof.

Preferred compounds of the immediately foregoing formula discovered to treat dyslipidemia in a mammalian subject include the following:

### Ac-c(Cys-Glu-His-D-4-Br-Phe-Arg-Trp-Gly-Cys)-(Pro)₂-Lys-Asp-NH₂;

### Ac-c(Cys-Glu-His-D-2-Nal-Arg-Trp-Ala-Cys)-(Pro)₂-Lys-Asp-NH₂;

### Ac-c(Cys-Glu-His-D-2-Nal-Arg-2-Nal-Ala-Cys)-(Pro)₂-Lys-Asp-NH₂;

Ac-c (Cys-Glu-His-D-2-Nal-Arg-1-Nal-Ala-Cys)-(Pr o)₂-Lys-Asp-NH₂;

### Ac-c(Cys-Glu-His-D-2-Nal-Arg-Bal-Ala-Cys)-(Pro)₂-Lys-Asp-NH₂;

### Ac-c(Cys-Glu-His-D-2-Nal-Arg-2-Nal-β-Ala-Cys)-(Pro)₂-Lys-Asp-NH₂; or

### Ac-c(Cys-Glu-His-D-2-Nal-Arg-2-Nal-Aib-Cys)-(Pro)₂-Lys-Asp-NH₂;

or pharmaceutically acceptable salts thereof.

The invention additionally provides a method to treat dyslipidemia in a mammalian subject by the administration of a therapeutically effective amount of a melanocortin receptor compound modified with a hydantoin moiety according to Formula (V), (VI) or (VII), and pharmaceutically acceptable salts, hydrates, solvates or prodrugs thereof.

According to a fifth embodiment, the invention provides a method to treat dyslipidemia in a mammalian subject by the administration of a therapeutically effective amount of a melanocortin receptor ligand according to the following formula (Formula (V)), pharmaceutically-acceptable salts, hydrates, solvates and/or prodrugs thereof (see International Patent Application Number PCT/US08/06675 incorporated herein by reference in its entirety):

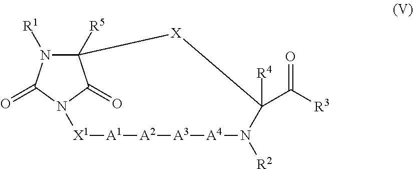

wherein

X is selected from the group consisting of —CH₂—S—S—CH₂—, —C(CH₃)₂—S—S—CH₂—, —CH₂—S—S—C(CH₃)₂—, —C(CH₃)₂—S—S—C(CH₃)₂—, —(CH₂)₂—S—S—CH₂—, —CH₂—S—S—(CH₂)₂—, —(CH₂)₂—S—S—(CH₂)₂—, —C(CH₃)₂—S—S—(CH₂)₂—, —(CH₂)₂—S—S—C(CH₃)₂—, —(CH₂)ₜ—C(O)—NR⁸—(CH₂)ᵣ— and —(CH₂)ᵣ—NR⁸—C(O)—(CH₂)ₜ—;

R¹ and R² each is, independently, H, (C₁-C₁₀)alkyl or substituted (C₁-C₁₀)alkyl;

R³ is —OH or —NH₂;

R⁴ and R⁵ each is, independently, H, (C₁-C₁₀)alkyl or substituted (C₁-C₁₀)alkyl;

X¹ is

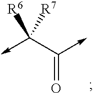

A¹ is H is, 2-Pal, 3-Pal, 4-Pal, (X¹,X²,X³,X⁴,X⁵)Phe, Taz, 2-Thi, 3-Thi or is deleted;

A² is D-Bal, D-1-Nal, D-2-Nal, D-Phe or D-(X¹,X²,X³,X⁴,X⁵)Phe;

A³ is Arg, hArg, Dab, Dap, Lys or Orn;

A⁴ is Bal, 1-Nal, 2-Nal, (X¹,X²,X³,X⁴,X⁵)Phe or Trp;

R⁶ and R⁷ each is, independently for each occurrence thereof, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl provided that R⁶ and R⁷ may be joined together to form a ring;

R⁸ is H, (C₁-C₁₀)alkyl or substituted (C₁-C₁₀)alkyl;

r is, independently for each occurrence thereof, 1, 2, 3, 4 or 5; and

t is, independently for each occurrence thereof, 1 or 2.

Preferrably, a compound according the foregoing fourmula found useful to treat dyslipidemia in a mammalian subject include compounds wherein X¹ is selected from the group consisting of:

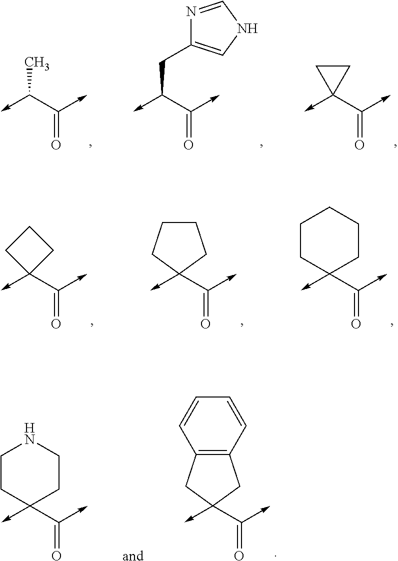

Representative embodiments of the foregoing class of compounds useful to treat dyslipidemia in a mammalian subject are as follows:

c[Hydantoin(C(O)-(Cys-D-Ala))-His-D-Phe-Arg-Trp-Cys]-NH₂;

c[Hydantoin(C(O)-(hCys-D-Ala))-His-D-Phe-Arg-Trp-Cys]-NH₂;

c[Hydantoin(C(O)-(Cys-D-Ala))-His-D-2-Nal-Arg-Trp-Cys]-NH₂;

c[Hydantoin(C(O)-(hCys-D-Ala))-His-D-2-Nal-Arg-Trp-Cys]-NH₂;

c[Hydantoin(C(O)-(Asp-D-Ala))-His-D-Phe-Arg-Trp-Lys]-NH₂;

c[Hydantoin(C(O)-(Asp-D-Ala))-His-D-Phe-Arg-Trp-Orn]-NH₂;

c[Hydantoin(C(O)-(Asp-D-Ala))-His-D-Phe-Arg-Trp-Dab]-NH₂;

c[Hydantoin(C(O)-(Asp-D-Ala))-His-D-Phe-Arg-Trp-Dap]-NH₂;

c[Hydantoin(C(O)-(Asp-His))-D-2-Nal-Arg-Trp-Lys]-NH₂;

c[Hydantoin(C(O)-(Asp-His))-D-Phe-Arg-Trp-Lys]-NH₂

c[Hydantoin(C(O)-(Asp-A3c))-D-Phe-Arg-Trp-Lys]-NH₂;

c[Hydantoin(C(O)-(Asp-A5c))-D-Phe-Arg-Trp-Lys]-NH₂;

c[Hydantoin(C(O)-(Asp-A6c))-D-Phe-Arg-Trp-Lys]-NH₂;

c[Hydantoin(C(O)-(Asp-A3c))-D-2-Nal-Arg-Trp-Lys]-NH₂;

c[Hydantoin(C(O)-(Asp-A5c))-D-2-Nal-Arg-Trp-Lys]-NH₂;

c[Hydantoin(C(O)-(Asp-A6c))-D-2-Nal-Arg-Trp-Lys]-NH₂;

c[Hydantoin(C(O)-(Asp-Aic))-D-Phe-Arg-Trp-Lys]-NH₂;

c[Hydantoin(C(O)-(Asp-Apc))-D-Phe-Arg-Trp-Lys]-NH₂;

c[Hydantoin(C(O)-(Asp-Aic))-D-2-Nal-Arg-Trp-Lys]-NH₂;

c[Hydantoin(C(O)-(Asp-Apc))-D-2-Nal-Arg-Trp-Lys]-NH₂;

c[Hydantoin-(C(O)-(Asp-Aic))-D-2-Nal-Arg-Trp-Lys]-NH₂,

c[Hydantoin-(C(O)-(Asp-Apc))-D-2-Nal-Arg-Trp-Lys]-NH₂

c[Hydantoin(C(O)-(Glu-D-Ala))-His-D-Phe-Arg-Trp-Orn]-NH₂;

c[Hydantoin(C(O)-(Glu-D-Ala))-His-D-Phe-Arg-Trp-Dab]-NH₂;

c[Hydantoin(C(O)-(Glu-D-Ala))-His-D-Phe-Arg-Trp-Dap]-NH₂, or

c[Hydantoin(C(O)-(Glu-His))-D-Phe-Arg-Trp-Dap]-NH₂

According to a sixth embodiment, the invention provides a method to treat dyslipidemia in a mammalian subject by the administration of a therapeutically effective amount of a melanocortin receptor compound according to Formula (VI), pharmaceutically-acceptable salts, hydrates, solvates and/or prodrugs thereof (see International Patent Application Number PCT/US08/06675 which is incorporated herein by reference in its entirety):

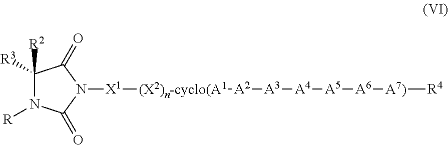

wherein

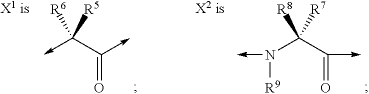

A¹ is Asp, Cys, D-Cys, Dab, Dap, Glu, Lys, Orn, Pen or D-Pen;

A² is an L- or D-amino acid;

A³ is H is, 2-Pal, 3-Pal, 4-Pal, (X¹,X²,X³,X⁴,X⁵)Phe, Taz, 2-Thi or 3-Thi;

A⁴ is D-Bal, D-1-Nal, D-2-Nal, D-Phe or D-(X¹,X²,X³,X⁴,X⁵)Phe;

A⁵ is Arg, hArg, Dab, Dap, Lys or Orn;

A⁶ is Bal, 1-Nal, 2-Nal, (X¹,X²,X³,X⁴,X⁵)Phe or Trp,

A⁷ is Asp, Cys, D-Cys, Dab, Dap, Glu, Lys, Orn, Pen or D-Pen;

R¹ is H, (C₁-C₁₀)alkyl or substituted (C₁-C₁₀)alkyl;

R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety;

R⁴ is CO₂H or C(O)NH₂;

R⁵ and R⁶ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety;

R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl; or R⁷ and R⁸ may be fused together form a cyclic moiety;

R⁹ is H, (C₁-C₁₀)alkyl or substituted (C₁-C₁₀)alkyl; and

n is, independently for each occurrence thereof, 1, 2, 3, 4, 5, 6 or 7;

or a pharmaceutically acceptable salt thereof.

A preferred class of compounds according to Formula (VI) useful to treat dyslipidemia in a mammalian subject are those compounds wherein:

A¹ is Cys;

A² is D-Ala, Asn, Asp, Gln, Glu or D-Phe;

A³ is H is;

A⁴ is D-2-Nal or D-Phe;

A⁵ is Arg;

A⁶ is Trp; and

A⁷ is Cys or Pen;

each of R¹, R², R³, and R⁹ is, independently, H;

R⁴ is C(O)NH₂;

each of R⁵ and R⁶ is, independently, H, (C₁-C₁₀)alkyl, (C₁-C₁₀)heteroalkyl, substituted (C₁-C₁₀)alkyl or substituted (C₁-C₁₀)heteroalkyl or R⁵ and R⁶ may be fused together form a cyclic moiety; and
each of R⁷ and R⁸ is, independently, H, (C₁-C₁₀)alkyl, (C₁-C₁₀)heteroalkyl, substituted (C₁-C₁₀)alkyl or substituted (C₁-C₁₀)heteroalkyl;
   or pharmaceutically acceptable salts thereof.

Preferred compounds of the immediately foregoing formula (Formula (VI)) useful to treat dyslipidemia in a mammalian subject include:

Hydantoin(C(O)-(Arg-Gly))-c(Cys-Glu-His-D-Phe-Arg-Trp-Cys)-NH₂;

Hydantoin(C(O)-(Nle-Gly))-c(Cys-Glu-His-D-Phe-Arg-Trp-Cys)-NH₂;

Hydantoin(C(O)-(Gly-Gly))-c(Cys-Glu-His-D-Phe-Arg-Trp-Cys)-NH₂;

Hydantoin(C(O)-(Nle-Gly))-c(Cys-D-Ala-His-D-Phe-Arg-Trp-Cys)-NH₂;

Hydantoin(C(O)-(Gly-Gly))-c(Cys-D-Ala-His-D-Phe-Arg-Trp-Cys)-NH₂;

Hydantoin(C(O)-(Nle-Gly))-c(Cys-D-Ala-His-D-Phe-Arg-Trp-Pen)-NH₂;

Hydantoin(C(O)-(Gly-Gly))-c(Cys-D-Ala-His-D-Phe-Arg-Trp-Pen)-NH₂;

Hydantoin(C(O)-(Ala-Gly))-c(Cys-D-Ala-His-D-Phe-Arg-Trp-Cys)-NH₂;

Hydantoin(C(O)-(D-Ala-Gly))-c(Cys-D-Ala-His-D-Phe-Arg-Trp-Cys)-NH₂;

Hydantoin(C(O)-(Aib-Gly))-c(Cys-D-Ala-His-D-Phe-Arg-Trp-Cys)-NH₂;

Hydantoin(C(O)-(Val-Gly))-c(Cys-D-Ala-His-D-Phe-Arg-Trp-Cys)-NH₂;

Hydantoin(C(O)-(Ile-Gly))-c(Cys-D-Ala-His-D-Phe-Arg-Trp-Cys)-NH₂;

Hydantoin(C(O)-(Leu-Gly))-c(Cys-D-Ala-His-D-Phe-Arg-Trp-Cys)-NH₂;

Hydantoin(C(O)-(Gly-Gly))-c(Cys-Glu-His-D-2-Nal-Arg-Trp-Cys)-NH₂;

Hydantoin(C(O)-(Nle-Gly))-c(Cys-Glu-His-D-2-Nal-Arg-Trp-Cys)-NH₂;

Hydantoin(C(O)-(D-Arg-Gly))-c(Cys-Glu-His-D-Phe-Arg-Trp-Cys)-NH₂;

Hydantoin(C(O)-(Arg-Gly))-c(Cys-Glu-His-D-Phe-Arg-Trp-Cys)-NH₂;

Hydantoin(C(O)-(D-Arg-Gly))-c(Cys-D-Ala-His-D-Phe-Arg-Trp-Cys)-NH₂;

Hydantoin(C(O)-(Arg-Gly))-c(Cys-D-Ala-His-D-Phe-Arg-Trp-Cys)-NH₂;

Hydantoin(C(O)-(D-Arg-Gly))-c(Cys-D-Ala-His-D-2-Nal-Arg-Trp-Cys)-NH₂;

Hydantoin(C(O)-(Arg-Gly))-c(Cys-D-Ala-His-D-2-Nal-Arg-Trp-Cys)-NH₂;

Hydantoin(C(O)-(Nle-Gly))-c(Cys-Glu-His-D-Phe-Arg-Trp-Cys)-NH₂;

Hydantoin(C(O)-(Gly-Gly))-c(Cys-Glu-His-D-Phe-Arg-Trp-Cys)-NH₂;

Hydantoin(C(O)-(Nle-Gly))-c(Cys-D-Ala-His-D-Phe-Arg-Trp-Cys)-NH₂;

Hydantoin(C(O)-(Gly-Gly))-c(Cys-D-Ala-His-D-Phe-Arg-Trp-Cys)-NH₂;

Hydantoin(C(O)-(Nle-Gly))-c(Cys-D-Ala-His-D-Phe-Arg-Trp-Pen)-NH₂;

Hydantoin(C(O)-(Gly-Gly))-c(Cys-D-Ala-His-D-Phe-Arg-Trp-Pen)-NH₂;

Hydantoin(C(O)-(Ala-Gly))-c(Cys-D-Ala-His-D-Phe-Arg-Trp-Cys)-NH₂;

Hydantoin(C(O)-(D-Ala-Gly))-c(Cys-D-Ala-His-D-Phe-Arg-Trp-Cys)-NH₂;

Hydantoin(C(O)-(Aib-Gly))-c(Cys-D-Ala-His-D-Phe-Arg-Trp-Cys)-NH₂;

Hydantoin(C(O)-(Val-Gly))-c(Cys-D-Ala-His-D-Phe-Arg-Trp-Cys)-NH₂;

Hydantoin(C(O)-(Ile-Gly))-c(Cys-D-Ala-His-D-Phe-Arg-Trp-Cys)-NH₂;

Hydantoin(C(O)-(Leu-Gly))-c(Cys-D-Ala-His-D-Phe-Arg-Trp-Cys)-NH₂;

Hydantoin(C(O)-(D-Arg-Gly))-c(Cys-Glu-His-D-Phe-Arg-Trp-Cys)-NH₂;

Hydantoin(C(O)-(Arg-Gly))-c(Cys-Glu-His-D-Phe-Arg-Trp-Cys)-NH₂;

Hydantoin(C(O)-(Arg-Gly))-c(Cys-D-Ala-His-D-Phe-Arg-Trp-Cys)-NH₂;

Hydantoin(C(O)-(D-Arg-Gly))-c(Cys-D-Ala-His-D-Phe-Arg-Trp-Cys)-NH₂;

Hydantoin(C(O)-(Ala-Nle))-c(Cys-Glu-His-D-Phe-Arg-Trp-Cys)-NH₂;

Hydantoin(C(O)-(Val-Nle))-c(Cys-Glu-His-D-Phe-Arg-Trp-Cys)-NH₂;

Hydantoin(C(O)-(Gly-Nle))-c(Cys-Glu-His-D-Phe-Arg-Trp-Cys)-NH₂;

Hydantoin(C(O)-(A6c-Nle))-c(Cys-D-Ala-His-D-Phe-Arg-Trp-Cys)-NH₂;

Hydantoin(C(O)-(Gly-Nle))-c(Cys-D-Ala-His-D-Phe-Arg-Trp-Cys)-NH₂;

Hydantoin(C(O)-(Ala-Nle))-c(Cys-D-Ala-His-D-Phe-Arg-Trp-Cys)-NH₂;

Hydantoin(C(O)-(D-Ala-Nle))-c(Cys-D-Ala-His-D-Phe-Arg-Trp-Cys)-NH₂;

Hydantoin(C(O)-(Val-Nle))-c(Cys-D-Ala-His-D-Phe-Arg-Trp-Cys)-NH₂;

Hydantoin(C(O)-(Leu-Nle))-c(Cys-D-Ala-His-D-Phe-Arg-Trp-Cys)-NH₂;

Hydantoin(C(O)-(Cha-Nle))-c(Cys-D-Ala-His-D-Phe-Arg-Trp-Cys)-NH₂;

Hydantoin(C(O)-(Aib-Nle))-c(Cys-D-Ala-His-D-Phe-Arg-Trp-Cys)-NH₂;

Hydantoin(C(O)-(Gly-Arg))-c(Cys-Glu-His-D-Phe-Arg-Trp-Cys)-NH₂;

Hydantoin(C(O)-(Gly-Arg))-c(Cys-Glu-His-D-2-Nal-Arg-Trp-Cys)-NH₂;

Hydantoin(C(O)-(Gly-Arg))-c(Cys-D-Ala-His-D-Phe-Arg-Trp-Cys)-NH₂;

Hydantoin(C(O)-(Gly-Arg))-c(Cys-D-Ala-His-D-2-Nal-Arg-Trp-Cys)-NH₂;

Hydantoin(C(O)-(Gly-D-Arg))-c(Cys-Glu-His-D-Phe-Arg-Trp-Cys)-NH₂;

Hydantoin(C(O)-(Gly-D-Arg))-c(Cys-Glu-His-D-Phe-Arg-Trp-Cys)-NH₂;

Hydantoin(C(O)-(Gly-D-Arg))-c(Cys-D-Ala-His-D-Phe-Arg-Trp-Cys)-NH₂;

Hydantoin(C(O)-(Gly-D-Arg))-c(Cys-D-Ala-His-D-2-Nal-Arg-Trp-Cys)-NH₂; or

Hydantoin(C(O)-(Nle-Ala))-c(Cys-Glu-His-D-Phe-Arg-Trp-Cys)-NH₂;

or pharmaceutically acceptable salts thereof.

In a seventh embodiment, the invention provides a method to treat dyslipidemia in a mammalian subject by the administration of a therapeutically effective amount of a melanocortin receptor ligand belonging to a class of cyclic peptide analogs that are ligands for the melanocortin receptors having a structure according to Formula (VII) as depicted below (see International Patent Application Number PCT/US08/06675 which is incorporated herein by reference in its entirety):

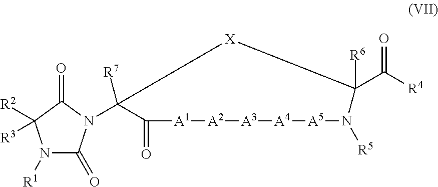

wherein

X is selected from the group consisting of —CH₂—S—S—CH₂—, —C(CH₃)₂—S—S—CH₂—, —CH₂—S—S—C(CH₃)₂—, —C(CH₃)₂—S—S—C(CH₃)₂—, —(CH₂)₂—S—S—CH₂—, —CH₂—S—S—(CH₂)₂, —(CH₂)₂—S—S—(CH₂)₂—, —C(CH₃)₂—S—S—(CH₂)₂—, —(CH₂)₂—S—S—C(CH₃)₂—, —(CH₂)ₜ—C(O)—NR⁸—(CH₂)ᵣ—, and —(CH₂)ᵣ—NR⁸—C(O)—(CH₂)ₜ—;

each of R¹ and R⁵ is, independently, H, (C₁-C₁₀)alkyl or substituted (C₁-C₁₀)alkyl;

each of R² and R³ is, independently, H, (C₁-C₁₀)alkyl, (C₁-C₁₀)heteroalkyl, aryl(C₁₀₅)alkyl, substituted (C₁-C₁₀)alkyl, substituted (C₁-C₁₀)heteroalkyl or substituted aryl(C₁-C₅)alkyl or R² and R³ may be fused together to form a ring;

R⁴ is OH or NH₂;

each of R⁶ and R⁷ is, independently, H, (C₁-C₁₀)alkyl or substituted (C₁-C₁₀)alkyl;

A¹ is an L- or D-amino acid or deleted;

A² is H is, 2-Pal, 3-Pal, 4-Pal, (X¹,X²,X³,X⁴,X⁵)Phe, Taz, 2-Thi or 3-Thi;

A³ is D-Bal, D-1-Nal, D-2-Nal, D-Phe or D-(X¹,X²,X³,X⁴,X⁵)Phe;

A⁴ is Arg, hArg, Dab, Dap, Lys or Orn;

A⁵ is Bal, 1-Nal, 2-Nal, (X¹,X²,X³,X⁴,X⁵)Phe or Trp;

r is, independently for each occurrence thereof, 1, 2, 3, 4 or 5; and

t is, independently for each occurrence thereof, 1 or 2;

or pharmaceutically acceptable salts thereof.

In the preferred aspect of the compounds according to Formula (VII) useful to treat dyslipidemia in a mammalian subject,

A¹ is Ala, D-Ala, Asn, Asp, Gln, Glu or Gly;

or pharmaceutically acceptable salts thereof.

Preferred compounds according to Formula (VII) useful in the treatment of dyslipidemia in a mammalian subject, include the following compounds:

c[Hydantoin(C(O)-(Nle-Cys))-D-Ala-His-D-Phe-Arg-Trp-Cys]-NH₂;

c[Hydantoin(C(O)-(Ala-Cys))-D-Ala-His-D-Phe-Arg-Trp-Cys]-NH₂;

c[Hydantoin(C(O)-(D-Ala-Cys))-D-Ala-His-D-Phe-Arg-Trp-Cys]-NH₂;

c[Hydantoin(C(O)-(Aib-Cys))-D-Ala-His-D-Phe-Arg-Trp-Cys]-NH₂;

c[Hydantoin(C(O)-(Val-Cys))-D-Ala-His-D-Phe-Arg-Trp-Cys]-NH₂;

c[Hydantoin(C(O)-(Abu-Cys))-D-Ala-His-D-Phe-Arg-Trp-Cys]-NH₂;

c[Hydantoin(C(O)-(Leu-Cys))-D-Ala-His-D-Phe-Arg-Trp-Cys]-NH₂;

c[Hydantoin(C(O)-(Ile-Cys))-D-Ala-His-D-Phe-Arg-Trp-Cys]-NH₂;

c[Hydantoin(C(O)-(Cha-Cys))-D-Ala-His-D-Phe-Arg-Trp-Cys]-NH₂;

c[Hydantoin(C(O)-(A6c-Cys))-D-Ala-His-D-Phe-Arg-Trp-Cys]-NH₂;

c[Hydantoin(C(O)-(Phe-Cys))-D-Ala-His-D-Phe-Arg-Trp-Cys]-NH₂;

c[Hydantoin(C(O)-(Gly-Cys))-D-Ala-His-D-Phe-Arg-Trp-Cys]-NH₂;

c[Hydantoin(C(O)-(Gly-Cys))-Glu-His-D-Phe-Arg-Trp-Cys]-NH₂; or

c[Hydantoin(C(O)-(Gly-Cys))-Glu-His-D-Phe-Arg-Trp-Cys]-NH₂;

or pharmaceutically acceptable salts thereof.

In an eighth embodiment, the present invention is directed to a method to treat dyslipidemia in a mammalian subject by the administration of a therapeutically effective amount of a melanocortin receptor ligand according to Formula (VIII) (see International Patent Application Number PCT/US08/07411, incorporated herein by reference in its entirety):

(R²R³)-A⁰-A¹-c(A²-A³-A⁴-A⁵-A⁶-A⁷-A⁸-A⁹)-A¹⁰-R¹ (VIII)

wherein:

A⁰ is an aromatic amino acid

A¹ is Acc, HN—(CH₂)ₘ—C(O), an L- or D-amino acid;

A² is Asp, Cys, D-Cys, hCys, D-hCys, Glu, Pen, or D-Pen;

A³ is Aib, Ala, β-Ala, Gaba, Gly or a D-amino acid;

A⁴ is H is, 2-Pal, 3-Pal, 4-Pal, (X¹,X²,X³,X⁴,X⁵)Phe, Taz, 2-Thi, or 3-Thi;

A⁵ is D-Bal, D-1-Nal, D-2-Nal, D-Phe, L-Phe, D-(X¹,X²,X³,X⁴,X⁵)Phe, L-Phe, D-Trp or D-(Et)Tyr;

A⁶ is Arg, hArg, Dab, Dap, Lys, Orn, or HN—CH((CH₂)ₙ—N(R⁴R⁵))—C(O);

A⁷ is Bal, D-Bal, Bip, D-Bip, 1-Nal, D-1-Nal, 2-Nal, D-2-Nal, or D-Trp;

A⁸ is Acc, Aha, Ahx, Ala, D-Ala, β-Ala, Apn, Gaba, Gly, HN—(CH₂)ₛ—C(O), or deleted;

A⁹ is Cys, D-Cys, hCys, D-hCys, Dab, Dap, Lys, Orn, Pen, or D-Pen;

A¹⁰ is Acc, HN—(CH₂)ₜ—C(O), L- or D-amino acid, or deleted;

R¹ is OH, or NH₂;

each of R² and R³ is, independently for each occurrence selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;

each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;

m is, independently for each occurrence, 1, 2, 3, 4, 5, 6 or 7;

n is, independently for each occurrence, 1, 2, 3, 4 or 5;

s is, independently for each occurrence, 1, 2, 3, 4, 5, 6, or 7;

t is, independently for each occurrence, 1, 2, 3, 4, 5, 6, or 7;

X¹, X², X³, X⁴, and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN; provided that

(I). when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;

(II). when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;

(III). when A² is Cys, D-Cys, hCys, D-hCys, Pen, or D-Pen, then A⁹ is Cys, D-Cys, hCys, D-hCys, Pen, or D-Pen;

(IV). when A² is Asp or Glu, then A⁹ is Dab, Dap, Orn, or Lys;

(V). when A⁸ is Ala or Gly, then A¹ is not Nle; or

pharmaceutically acceptable salts thereof.

A preferred group of compounds of the immediate foregoing formula useful to treat dyslipidemia in a mammalian subject is wherein

A⁰ is 1-Nal, 2-Nal, H is, Pff, Phe, Trp, or Tyr;

A¹ is Arg;

A² is Cys;

A³ is D-Ala;

A⁴ is H is;

A⁵ is D-Phe

A⁶ is Arg;

A⁷ is Trp;

A⁸ is deleted;

A⁹ is Cys; and

A¹⁰ is deleted;

or pharmaceutically acceptable salts thereof.

Preferred compounds of the immediately foregoing group of compounds is which are useful to treat dyslipidemia in a mammalian subject of the formula:

### Ac-Tyr-Arg-c(Cys-D-Ala-His-D-Phe-Arg-Trp-Cys)-NH₂;

### Ac-2-Nal-Arg-c(Cys-D-Ala-His-D-Phe-Arg-Trp-Cys)-NH₂;

### Ac-1-Nal-Arg-c(Cys-D-Ala-His-DPhe-Arg-Trp-Cys)-NH₂;

### Ac-Phe-Arg-c(Cys-D-Ala-His-D-Phe-Arg-Trp-Cys)-NH₂;

### Ac-Trp-Arg-c(Cys-D-Ala-His-D-Phe-Arg-Trp-Cys)-NH₂;

### Ac-Pff-Arg-c(Cys-D-Ala-His-D-Phe-Arg-Trp-Cys)-NH₂;

### H-His-Arg-c(Cys-D-Ala-His-D-Phe-Arg-Trp-Cys)-NH₂; or

### Ac-His-Arg-c(Cys-D-Ala-His-D-Phe-Arg-Trp-Cys)-NH₂;

or pharmaceutically acceptable salts thereof.

In yet another preferred embodiment, the compound or compounds of Formula (I), (II), (III), (IV), (V), (VI), (VII) or (VIII) as defined hereinabove, which are useful to treat dyslipidemia in a mammalian subject or a pharmaceutically acceptable salt thereof, are provided to said subject in need in a composition with a pharmaceutically acceptable carrier or diluent.

In preferred embodiment, the invention provides a method of treating dyslipidemia in a subject in need thereof, comprising peripheral administration of an effective amount of a melanocortin receptor 4 agonist to treat the dyslipidemic subject in need thereof.

In one aspect, the melanocortin receptor 4 agonist according to any one of the compound or compounds of Formula (I), (II), (III), (IV), (V), (VI), (VII) or (VIII), or a pharmaceutically acceptable salt thereof, as defined herein are useful to treat dyslipidemia in the subject in need thereof.

In one preferred aspect, the melanocortin receptor 4 agonist useful to treat dyslipidemia in the subject in need thereof, is Ac-Arg-c(Cys-D-Ala-His-D-Phe-Arg-Trp-Cys)-NH₂ or a pharmaceutically acceptable salt thereof.

In one preferred aspect, the melanocortin receptor 4 agonist useful to treat dyslipidemia in the subject in need thereof, Hydantoin(C(O)-(Arg-Gly))-c(Cys-Glu-His-D-Phe-Arg-Trp-Cys)-NH₂ or a pharmaceutically acceptable salt thereof.

Other melanocortin ligands suitable for use in the practice of the invention include compounds, compositions or combinations thereof disclosed in:

U.S. Pat. No. 7,517,854 (incorporated herein by reference in its entirety, particularly for compounds, compositions or combinations thereof disclosed within);

U.S. Pat. No. 7,501,525 (incorporated herein by reference in its entirety, particularly for compounds, compositions or combinations thereof disclosed within);

U.S. Pat. No. 7,495,009 (incorporated herein by reference in its entirety, particularly for compounds, compositions or combinations thereof disclosed within);

U.S. Pat. No. 7,473,760 (incorporated herein by reference in its entirety, particularly for compounds, compositions or combinations thereof disclosed within);

U.S. Pat. No. 7,456,184 (incorporated herein by reference in its entirety, particularly for compounds, compositions or combinations thereof disclosed within);

U.S. Pat. No. 7,419,990 (incorporated herein by reference in its entirety, particularly for compounds, compositions or combinations thereof disclosed within);

U.S. Pat. No. 7,417,027 (incorporated herein by reference in its entirety, particularly for compounds, compositions or combinations thereof disclosed within);

U.S. Pat. No. 7,414,057 (incorporated herein by reference in its entirety, particularly for compounds, compositions or combinations thereof disclosed within);

U.S. Pat. No. 7,368,453 (incorporated herein by reference in its entirety, particularly for compounds, compositions or combinations thereof disclosed within);

U.S. Pat. No. 7,354,923 (incorporated herein by reference in its entirety, particularly for compounds, compositions or combinations thereof disclosed within);

U.S. Pat. No. 7,345,144 (incorporated herein by reference in its entirety, particularly for compounds, compositions or combinations thereof disclosed within);

U.S. Pat. No. 7,342,089 (incorporated herein by reference in its entirety, particularly for compounds, compositions or combinations thereof disclosed within);

U.S. Pat. No. 7,329,673 (incorporated herein by reference in its entirety, particularly for compounds, compositions or combinations thereof disclosed within);

U.S. Pat. No. 7,326,707 (incorporated herein by reference in its entirety, particularly for compounds, compositions or combinations thereof disclosed within);

U.S. Pat. No. 7,314,879 (incorporated herein by reference in its entirety, particularly for compounds, compositions or combinations thereof disclosed within);

U.S. Pat. No. 7,307,063 (incorporated herein by reference in its entirety, particularly for compounds, compositions or combinations thereof disclosed within);

U.S. Pat. No. 7,291,619 (incorporated herein by reference in its entirety, particularly for compounds, compositions or combinations thereof disclosed within);

U.S. Pat. No. 7,276,520 (incorporated herein by reference in its entirety, particularly for compounds, compositions or combinations thereof disclosed within);

U.S. Pat. No. 7,189,755 (incorporated herein by reference in its entirety, particularly for compounds, compositions or combinations thereof disclosed within);

U.S. Pat. No. 7,189,727 (incorporated herein by reference in its entirety, particularly for compounds, compositions or combinations thereof disclosed within);

U.S. Pat. No. 7,186,715 (incorporated herein by reference in its entirety, particularly for compounds, compositions or combinations thereof disclosed within);

U.S. Pat. No. 7,169,777 (incorporated herein by reference in its entirety, particularly for compounds, compositions or combinations thereof disclosed within);

U.S. Pat. No. 7,160,886 (incorporated herein by reference in its entirety, particularly for compounds, compositions or combinations thereof disclosed within);

U.S. Pat. No. 7,157,463 (incorporated herein by reference in its entirety, particularly for compounds, compositions or combinations thereof disclosed within);

U.S. Pat. No. 7,115,607 (incorporated herein by reference in its entirety, particularly for compounds, compositions or combinations thereof disclosed within);

U.S. Pat. No. 7,049,398 (incorporated herein by reference in its entirety, particularly for compounds, compositions or combinations thereof disclosed within);

U.S. Pat. No. 7,034,033 (incorporated herein by reference in its entirety, particularly for compounds, compositions or combinations thereof disclosed within);

U.S. Pat. No. 6,977,264 (incorporated herein by reference in its entirety, particularly for compounds, compositions or combinations thereof disclosed within);

U.S. Pat. No. 6,960,582 (incorporated herein by reference in its entirety, particularly for compounds, compositions or combinations thereof disclosed within);

U.S. Pat. No. 6,794,489 (incorporated herein by reference in its entirety, particularly for compounds, compositions or combinations thereof disclosed within);

U.S. Pat. No. 6,699,873 (incorporated herein by reference in its entirety, particularly for compounds, compositions or combinations thereof disclosed within);

U.S. Pat. No. 6,579,968 (incorporated herein by reference in its entirety, particularly for compounds, compositions or combinations thereof disclosed within);

U.S. Pat. No. 5,731,408 (incorporated herein by reference in its entirety, particularly for compounds, compositions or combinations thereof disclosed within);

U.S. Patent Application Publication No. 20090069224 (incorporated herein by reference in its entirety, particularly for compounds, compositions or combinations thereof disclosed within);

U.S. Patent Application Publication No. 20080234289 (incorporated herein by reference in its entirety, particularly for compounds, compositions or combinations thereof disclosed within);

U.S. Patent Application Publication No. 20080070921 (incorporated herein by reference in its entirety, particularly for compounds, compositions or combinations thereof disclosed within);

U.S. Patent Application Publication No. 20070155670 (incorporated herein by reference in its entirety, particularly for compounds, compositions or combinations thereof disclosed within);

U.S. Patent Application Publication No. 20060287332 (incorporated herein by reference in its entirety, particularly for compounds, compositions or combinations thereof disclosed within);

U.S. Patent Application Publication No. 20060287331 (incorporated herein by reference in its entirety, particularly for compounds, compositions or combinations thereof disclosed within);

U.S. Patent Application Publication No. 20060287330 (incorporated herein by reference in its entirety, particularly for compounds, compositions or combinations thereof disclosed within);

U.S. Patent Application Publication No. 20060281784 (incorporated herein by reference in its entirety, particularly for compounds, compositions or combinations thereof disclosed within);

U.S. Patent Application Publication No. 20060173036 (incorporated herein by reference in its entirety, particularly for compounds, compositions or combinations thereof disclosed within);

U.S. Patent Application Publication No. 20060111281 (incorporated herein by reference in its entirety, particularly for compounds, compositions or combinations thereof disclosed within);

U.S. Patent Application Publication No. 20060014676 (incorporated herein by reference in its entirety, particularly for compounds, compositions or combinations thereof disclosed within);

U.S. Patent Application Publication No. 20060014194 (incorporated herein by reference in its entirety, particularly for compounds, compositions or combinations thereof disclosed within);

U.S. Patent Application Publication No. 20050176728 (incorporated herein by reference in its entirety, particularly for compounds, compositions or combinations thereof disclosed within);

U.S. Patent Application Publication No. 20050164914 (incorporated herein by reference in its entirety, particularly for compounds, compositions or combinations thereof disclosed within);

U.S. Patent Application Publication No. 20050124636 (incorporated herein by reference in its entirety, particularly for compounds, compositions or combinations thereof disclosed within);

U.S. Patent Application Publication No. 20050038230 (incorporated herein by reference in its entirety, particularly for compounds, compositions or combinations thereof disclosed within);

U.S. Patent Application Publication No. 20050037951 (incorporated herein by reference in its entirety, particularly for compounds, compositions or combinations thereof disclosed within);

U.S. Patent Application Publication No. 20040106682 (incorporated herein by reference in its entirety, particularly for compounds, compositions or combinations thereof disclosed within);

U.S. Patent Application Publication No. 20040224957 (incorporated herein by reference in its entirety, particularly for compounds, compositions or combinations thereof disclosed within);

U.S. Patent Application Publication No. 20040167201 (incorporated herein by reference in its entirety, particularly for compounds, compositions or combinations thereof disclosed within);

U.S. Patent Application Publication No. 20040157264 (incorporated herein by reference in its entirety, particularly for compounds, compositions or combinations thereof disclosed within);

U.S. Patent Application Publication No. 20040152134 (incorporated herein by reference in its entirety, particularly for compounds, compositions or combinations thereof disclosed within);

U.S. Patent Application Publication No. 20040024211 (incorporated herein by reference in its entirety, particularly for compounds, compositions or combinations thereof disclosed within);

U.S. Patent Application Publication No. 20020143141 (incorporated herein by reference in its entirety, particularly for compounds, compositions or combinations thereof disclosed within).

In other aspects of the invention, administration of a compound or compostion comprising a compound or pharmaceutical salt of a compound of the invention useful to treat dyslipidemia, is continuous, hourly, four times daily, three time daily, twice daily, once daily, once every other day, twice weekly, once weekly, once every two weeks, once a month, or once every two months, or longer.

The dyslipidemic subject in need of treatment may be obese, overweight, of normal weight or lean. The obese, overweight, normal weight or lean subject may suffer from type II diabetes. The preferred administration of a compound or compostion comprising a compound or pharmaceutical salt of a compound of the invention useful to treat dyslipidemia is peripheral administration. Examples of peripheral administration include oral, subcutaneous, intraperitoneal, intramuscular, intravenous, rectal, transdermal or intranasal forms of administration.

### BRIEF DESCRIPTION OF THE DRAWINGS

FIG. 1: Dose-related decrease in body weight gain induced by sub-cutaneous (sc) infusion of Compound A in obese Zucker rats. Data is presented as the difference in mean body weight gain from vehicle treated group (g)±standard error of the mean (shaded area is the mean standard error of the vehicle group).

FIG. 2. Dose-related decrease in food intake induced by sc infusion of Compound A in obese Zucker rats. Data is presented as the difference in mean body weight gain from vehicle treated group (g)±standard error of the mean (shaded area is the mean standard error of the vehicle group).

FIG. 3. Dose-related decrease in triglycerides induced by sc infusion of Compound A. Data is presented as serum triglyceride level (mg/dL)±standard error of the mean.

FIG. 4. Dose-related decrease in cholesterol induced by sc infusion of Compound A. Data is presented as serum cholesterol level (g/dL)±standard error of the mean.

FIG. 5. Decrease in free fatty acids induced by sc infusion of Compound A. Data is presented as serum free fatty acid level (mmole/L)±standard error of the mean.

### DETAILED DESCRIPTION OF THE INVENTION

Recent studies have reported that staggering numbers of people world wide are overweight and suffering a wide variety of serious and expensive health problems. According the World Health Organization (as reported in Kouris-Blazos et al., Asia Pac. J. Clin. Nutr., 2007, 16:329-338), an estimated 1 billion people throughout the world are overweight and an estimated 300 million of these are obese. An estimated 22 million children under the age of 5 are severely overweight and in the European Union alone, the number of children who are overweight is expected to rise by 1.3 million children per year (Kosti et al., 2006, Cent. Eur. J. Public Health, 14:151-159). Obesity, as defined by the Statistical Bulletin provided by the Metropolitan Life Insurance Co., (1959, 40:1), is a condition in which a person is approximately 20-25% over normal body weight. Alternatively, an individual is considered obese if the person has a body mass index of greater than 25% over normal or greater than 30% over normal with risk factors (see Bray et al., Diabetes/Metabolism Review, 1988, 4:653-679 or Flynn et al., Proc. Nutritional Society, 1991, 50:413). One of the main causes for obesity is the consumption of a high caloric diet (Riccardi et al., Clin. Nutr., 2004, 23:447-456).

Diabetes is a chronic, debilitating disease afflicting many overweight and obese people. It is estimated that 20.8 million people in the United States alone have diabetes and more than 6 million more additional cases remain undiagnosed (Cornell, Manag. Care Pharm., 2007, 13:S11-5). Type 2 diabetes (also referred to herein as type II diabetes) is a chronic disease characterized by insulin resistance, impaired insulin secretion and hyperglycemia. Worldwide, type II diabetes is believed to affect approximately 171 million people, imparting numerous microvascular and macrovascular complications resulting in morbidity and mortality (Mudaliar, Indian J. Med. Res., 2007, 125:275-296). Mudaliar further notes that despite the availability of anti-hyperglycaemic agents available, control of glucose remains elusive in many patients.

Dyslipidemia is a condition in which may also result when energy consumption far exceeds the expenditure of energy. The unused energy is conserved in the form of fat (i.e., triacylglycerol (TG)) which accumulates in adipose tissue leading to the accumulation of excess body weight. Often times, the excess TGs accumulate in large vacuoles in the liver cells, a condition known as fatty liver disease (FLD) or heptatic steatosis. In the early stages of FLD, the vesicles are small (microvesicular) but can enlarge and crowd the cell (macrovesicular). In the past, the majority of FLD cases were associated with alcohol consumption but FLD is becoming more common without this factor (non-alcoholic fatty liver disease or NAFLD). FLD is now categorized into two broad groups ALFD (alcoholic FLD) and NAFLD, typically associated with overweight and obese subjects (see Reddy et al., Am. J. Physiol. Gastrointest. Liver Physiol., 2006, 290:G852-G858).

It is estimated that 20-35% of the general adult population in the US have hepatic steatosis and that approximately 10% of these cases will advance to NAFLD. In contrast, in the obese population, it is estimated that 75% have steatosis and that about 35% or more of this population will advance to full NAFLD. Other causes of NAFLD include parenteral nutrition, gastric bypass surgery and certain disorders associated with fatty acid metabolism. NAFLD typically worsens and progresses from the early stages of simple fat accumulation in the liver (hepatic steatosis) to nonalcoholic steatohepatitis (NASH) to steatonecrosis to steatonecrosis complicated by fibrosis leading to cirrhosis of the liver.

Additional complications of NAFLD or AFLD include but are not limited to, cell death, inflammation, lobular inflammation, ballooning degeneration of liver tissue, hepatocellular regeneration, stellate cell activation, fibrogenesis, cirrhosis and hepatocellular carcinoma. In essence, excess energy consumption coupled with reduced energy combustion (due, for example, to defective fatty acid oxidation in the liver), can trigger hepatic steatosis which can ultimately lead to cirrhosis, liver cancer and death.

Melanocortins are proposed to play a large role in energy metabolism and homeostasis. Melanocortins cleaved from the POMC precursor exert their effects by binding to members of the melanocortin receptor family located in the brain. The major effect of melanocortin in the brain is to reduce food intake however, it has also been shown that melanocortin agonists or antagonists injected directly into the cerebral ventricle affect insulin actions in the periphery while food was withdrawn or while food intake was kept constant (see Schwartz et al., Nature, 2000, 404:661-671; Seeley et al., Ann. Rev. Nutr., 2004, 24:133-149; Cone et al., Recent Prog. Horm. Res., 1996, 51:287-317; Heijbor et al., Diabetologia, 2005, 48:1621-1626; Obici et al., J. Clin. Inv., 2001, 108:1079-1085). Banno et al., (FEBS letters, 2007, 581:1131-1136) demonstrated that intracerebral injections of a melanocortin agonist to DIO rats decreased the size of and increased the number of adipocytes in white adipose tissue and decreased triglycerides content in the liver.

Considering the large numbers of overweight subjects in need of treatment, intracerebral administration is an unlikely means to disperse medicaments to patients. There is a need in the art, therefore, to identify melanocortin agonists and antagonists suitable for peripheral administration to affect parameters of insulin action and energy metabolism such as cellular characteristics of white adipose tissue, triglyceride levels and the like.

### Nomenclature and Abbreviations

As used herein, an “obese subject” or mammal is characterized as having a body weight approximately 20% or greater than the normal body weight for said subject. Normal body weight may be determined by a comparison of the weight of the subject at a prior point in time or by a comparison of the weight of the subject as compared to averages of other subjects of a similar age and/or condition.

As used herein, an “overweight subject” or mammal is characterized as having a body weight approximately 5% greater to approximately 20% greater than the normal body weight for said subject. Normal body weight may be determined by a comparison of the weight of the subject at a prior point in time or by a comparison of the weight of the subject as compared to averages of other subjects of a similar age and/or condition.

As used herein, a “normal subject” or mammal is characterized as having a body weight up to approximately 5% greater than to approximately 5% less than the normal body weight for said subject. Normal body weight may be determined by a comparison of the weight of the subject at a prior point in time or by a comparison of the weight of the subject as compared to averages of other subjects of a similar age and/or condition.

As used herein, a “lean subject” or mammal is characterized as having a body weight approximately 5% to 30% or even to 50% less than the normal body weight for said subject. Normal body weight may be determined by a comparison of the weight of the subject at a prior point in time or by a comparison of the weight of the subject as compared to averages of other subjects of a similar age and/or condition.

As used herein, the terms “treat”, “treating” and “treatment” include palliative, curative and prophylactic treatment.

As used herein, “measurable” means the biologic effect is both reproducible and significantly different from the baseline variability of the assay.

As used herein, “dyslipidemia” refers to a biological condition in which lipid metabolism is abnormal, including lipoprotein overproduction or underproduction. Dyslipidemia in which lipoproteins are over-produced typically results in an elevation of total cholesterol, low-density lipoprotein (LDL) cholesterol and triglycerides concentrations, with a concomitant decrease in high-density lipoprotein (HDL) cholesterol concentration in the blood.

As used herein, “fatty liver disease” or “hepatic steatosis” refers to a condition in which the liver has accumulated greater than normal levels of triglycerides in the hepatocytes of the liver. The triglycerides are contained in either or both micro- or macrovesicular vacuoles within the hepatocyte cells. The diagnosis is made when the lipid content of the liver exceeds 5010% by weight. FLD may or may not be associated with consumption of alcohol (see Reddy et al., Am. J. Physiol. Gastrointest. Liver Physiol., 2006, 290:G852-G858).

As used herein, “alcoholic fatty liver disease” refers to a condition of fatty liver disease in which the subject consumes on average, greater than 20 grams per day of alcohol. AFLD develops in essentially all individuals who consume approximately 60 or more grams of alcohol per day. AFLD can occur after the ingestion of moderate to large amounts of alcohol for even a short period of time. The subject may or may not be overweight or obese.

As used herein, “non-alcoholic fatty liver disease” refers to a condition of fatty liver disease in which the subject consumes on average, less than 20 grams per day of alcohol. The subject may or may not be overweight or obese.

As used herein, “nonalcoholic steatohepatitis” or NASH refers to that stage of the development of NA fatty liver disease in which macrovesicles of fat have developed accompanied by lobular inflammation in the liver. Steatohepatitis, in which macrovesicles of fat have developed accompanied by lobular inflammation in the liver, may also occur in alcoholic fatty liver disease.

As used herein, “steatonecrosis” refers to that stage of NA fatty liver disease in which macrovesicles of fat have developed accompanied by lobular inflammation and ballooning degeneration in the liver. Further development of NAFLD from the level of steatonecrosis includes the development of fibrosis in addition to the presence of macrovesicles of fat, inflammation and ballooning degeneration in the liver. Steatonecrosis, in which macrovesicles of fat have developed accompanied by lobular inflammation and ballooning degeneration in the liver, we well as the development of fibrosis in addition to the presence of macrovesicles of fat, inflammation and ballooning degeneration in the liver may also occur in alcoholic fatty liver disease.

As used herein, peripherial administration includes all forms of administration of a compound or a composition comprising a compound of the instant invention which excludes intracranial administration. Examples of peripheral administration include, but are not limited to, oral, parenteral (e.g., intramuscular, intraperitoneal, intravenous or subcutaneous injection, implant and the like), nasal, vaginal, rectal, sublingual or topical routes of administration, including transdermal patch applications and the like.

A “subject”, as used herein and throughout this application, refers to a mammalian or non-mammalian animal including, for example and without limitation, a human, a rat, a mouse or farm animal. Reference to a subject does not necessarily indicate the presence of a disease or disorder. The term “subject” includes, for example, a mammalian or non-mammalian animal being dosed with a melanocortin analog as part of an experiment, a mammalian or non-mammalian animal being treated to help alleviate a disease or disorder, and a mammalian or non-mammalian animal being treated prophylactically to retard or prevent the onset of a disease or disorder. Subject mammals may be human subjects of any age, such as an infant, a child, an adult or an elderly adult.

A “therapeutically acceptable amount” of a compound or composition of the invention, regardless of the formulation or route of administration, is that amount which elicits a desired biological response in a subject. The biological effect of the therapeutic amount may occur at and be measured at many levels in an organism. For example, the biological effect of the therapeutic amount may occur at and be measured at the cellular level by measuring the response at a receptor which binds melanocortin and/or a melanocortin analog, or the biological effect of the therapeutic amount may occur at and be measured at the system level, such as effecting an increase/decrease in the levels of insulin. The biological effect of the therapeutic amount may occur at and be measured at the organism level, such as the alleviation of a symptom(s) or progression of a disease or condition in a subject. A therapeutically acceptable amount of a compound or composition of the invention, regardless of the formulation or route of administration, may result in one or more biological responses in a subject. In the event that the compound or composition of the invention is subject to testing in an in vitro system, a therapeutically acceptable amount of the compound or composition may be viewed as that amount which gives a measurable response in the in vitro system of choice.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

The compounds of the invention useful for the treatment of dyslipidemia may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The compounds of the invention useful for the treatment of dyslipidemia may exist in one or more tautomeric forms. All tautomers and mixtures thereof are included in the scope of the present invention. For example, a claim to 2-hydroxypyridinyl would also cover its tautomeric form, a-pyridonyl.

Unless defined otherwise, all technical and scientific terms used herein have the same meaning as commonly understood by one of ordinary skill in the art to which this invention belongs. Also, all publications, patent applications, patents and other references mentioned herein are incorporated by reference in their entirety.

| Symbol | Meaning |
|---|---|
| Abu | α-aminobutyric acid |
| Ac | acyl group |
| Acc | 1-amino-1-cyclo(C₃-C₉)alkyl carboxylic acid |
| A3c | 1-amino-1-cyclopropanecarboxylic acid |
| A4c | 1-amino-1-cyclobutanecarboxylic acid |
| A5c | 1-amino-1-cyclopentanecarboxylic acid |
| A6c | 1-amino-1-cyclohexanecarboxylic acid |
| Aha | 7-aminoheptanoic acid |
| Ahx | 6-aminohexanoic acid |
| Aib | α-aminoisobutyric acid |
| Aic | 2-aminoindan-2-carboxylic acid |
| Ala or A | alanine |
| β-Ala | β-alanine |
| Apc | denotes the structure: |
| | 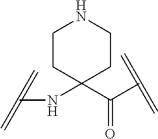 |
| Apn | 5-aminopentanoic acid (HN—(CH2)₄—C(O) |
| Arg or R | arginine |
| hArg | homoarginine |
| Asn or N | asparagine |
| Asp or D | aspartic acid |
| Bal | 3-benzothienylalanine |
| Bip | 4,4′-biphenylalanine, represented by the structure |
| | 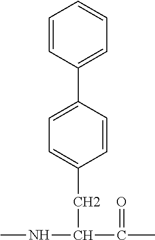 |
| Bpa | 4-benzoylphenylalanine |
| 4-Br-Phe | 4-bromo-phenylalanine |
| Cha | β-cyclohexylalanine |
| hCha | homo-cyclohexylalanine |
| Chg | cyclohexylglycine |
| Cys or C | cysteine |
| hCys | homocysteine |
| Dab | 2,4-diaminobutyric acid |
| Dap | 2,3-diaminopropionic acid |
| Dip | β,β-diphenylalanine |
| Doc | 8-amino-3,6-dioxaoctanoic acid with the structure of: |
| | 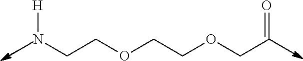 |
| 2-Fua | β-(2-furyl)-alanine |
| Gaba | 4-aminobutyric acid |
| Gln or Q | glutamine |
| Glu or E | glutamic acid |
| Gly or G | glycine |
| His or H | histidine |
| 3-Hyp | trans-3-hydroxy-L-proline, i.e., |
| | (2S, 3S)-3-hydroxypyrrolidine-2-carboxylic acid |
| 4-Hyp | 4-hydroxyproline, i.e., (2S, 4R)-4- |
| | hydroxypyrrolidine-2-carboxylic acid |
| Ile or I | isoleucine |
| Leu or L | leucine |
| hLeu | homoleucine |
| Lys or K | lysine |
| Met or M | methionine |
| β-hMet | β-homomethionine |
| 1-Nal | β-(1-naphthyl)alanine: |
| 2-Nal | β-(2-naphthyl)alanine |
| Nip | nipecotic acid |
| Nle | norleucine |
| Oic | octahydroindole-2-carboxylic acid |
| Orn | ornithine |
| 2-Pal | β-(2-pyridiyl)alanine |
| 3-Pal | β-(3-pyridiyl)alanine |
| 4-Pal | β-(4-pyridiyl)alanine |
| Pen | penicillamine |
| Pff | (S)-pentafluorophenylalanine |
| Phe or F | phenylalanine |
| hPhe | homophenylalanine |
| Pro or P | proline |
| hPro | homoproline |
| Ser or S | serine |
| Tle | tert-Leucine |
| Taz | β-(4-thiazolyl)alanine |
| 2-Thi | β-(2-thienyl)alanine |
| 3-Thi | β-(3-thienyl)alanine |
| Thr or T | threonine |
| Trp or W | tryptophan |
| Tyr or Y | tyrosine |
| D-(Et)Tyr | has a structure of |
| | 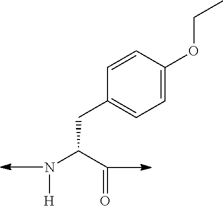 |
| Val or V | valine |

Certain other abbreviations used herein are defined as follows:

| | |
|---|---|
| Boc: | tert-butyloxycarbonyl |
| Bzl: | benzyl |
| DCM: | dichloromethane |
| DIC: | N, N-diisopropylcarbodiimide |
| DIEA: | diisopropylethyl amine |
| Dmab: | 4-{N-(1-(4,4-dimethyl-2,6-dioxocyclohexylidene)- |
| | 3-methylbutyl)-amino}benzyl |
| DMAP: | 4-(dimethylamino)pyridine |
| DMF | dimethylformamide |
| DNP: | 2,4-dinitrophenyl |
| Fm: | fluorenylmethyl |
| Fmoc: | fluorenylmethyloxycarbonyl |
| For: | formyl |
| HBTU: | 2-(1H-benzotriazole-1-yl)-1,1,3,3- |
| | tetramethyluronium hexafluorophosphate |
| cHex | cyclohexyl |
| HOAT: | O-(7-azabenzotriazol-1-yl)-1,1,3,3- |
| | tetramethyluronium hexafluorophosphate |
| HOBt: | 1-hydroxy-benzotriazole |
| MBHA | 4-methylbenzhydrylamine |
| Mmt: | 4-methoxytrityl |
| NMP: | N-methylpyrrolidone |
| O-tBu | oxy-tert-butyl |
| Pbf: | 2,2,4,6,7-pentamethyldihydrobenzofuran-5-sulfonyl |
| PyBroP | bromo-tris-pyrrolidino-phosphonium |
| | hexafluorophosphate |
| tBu: | tert-butyl |
| TIS: | triisopropylsilane |
| TOS: | tosyl |
| Trt | trityl |
| TFA: | trifluoro acetic acid |
| TFFH: | tetramethylfluoroforamidinium hexafluorophosphate |
| Z: | benzyloxycarbonyl |

Unless otherwise indicated, with the exception of the N-terminal amino acid, all abbreviations (e.g. Ala) of amino acids in this disclosure stand for the structure of —NH—C(R)(R′)—CO—, wherein R and R′ each is, independently, hydrogen or the side chain of an amino acid (e.g., R═CH₃ and R′═H for Ala), or R and R′ may be joined to form a ring system.

For the N-terminal amino acid, the abbreviation stands for the structure of:

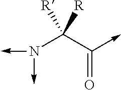

The designation “NH₂” in e.g., Ac-Nle-c(Cys-D-Ala-His-D-Phe-Arg-Trp-Cys)-NH₂, indicates that the C-terminus of the peptide is amidated. Ac-Nle-c(Cys-D-Ala-His-D-Phe-Arg-Trp-Cys), or alternatively Ac-Nle-c(Cys-D-Ala-His-D-Phe-Arg-Trp-Cys)-OH, indicates that the C-terminus is the free acid.

“-c(Cys-Cys)-” or “-cyclo(Cys-Cys)-” denotes the structure:

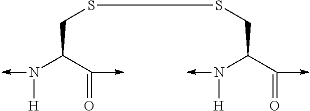

“-c(Cys-Pen)-” or “-cyclo(Cys-Pen)-” denotes the structure:

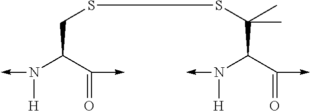

“-c(Asp-Lys)-” or “-cyclo(Asp-Lys)-” denotes the structure:

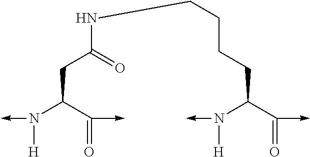

Applicants have devised the following shorthand used in naming the specific embodiments and/or species:

“HydantoinC(O)-(A^{a}-A^{b})” denotes the structure:

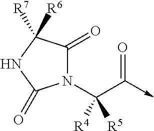

wherein amino acid “A^{a}” has the structure:

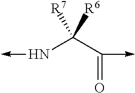

and

amino acid “Ab” the structure:

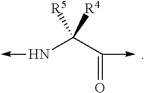

For example, a compound represented as “c[Hydantoin(C(O)-(Cys-A^{b})) A¹-A²-A³-A⁴-Cys]-” would have the following the structure:

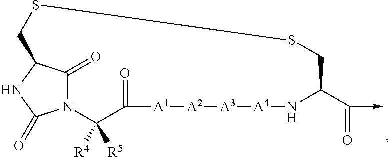

whereas a compound represented as “c[Hydantoin(C(O)-(A^{b}-Cys)) A¹-A²-A³-A⁴-Cys]-” would have the structure:

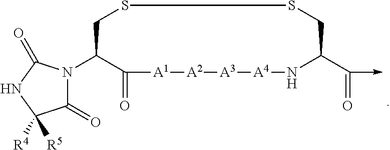

For further guidance, “c[Hydantoin(C(O)-(Asp-A^{b})) A¹-A²-A³-A⁴-Lys]-” represents the following compound:

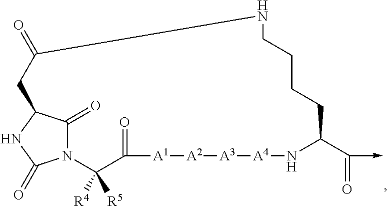

whereas “c[Hydantoin(C(O)-(Dap-A^{b})) A¹-A²-A³-A⁴-Asp]-” has the following formula:

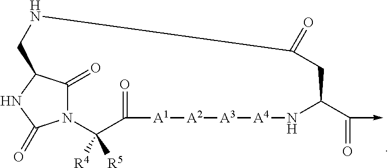

“Acyl” refers to R″—C(O)—, where R″ is H, alkyl, substituted alkyl, heteroalkyl, substituted heteroalkyl, alkenyl, substituted alkenyl, aryl, alkylaryl, or substituted alklyaryl, and is indicated in the general formula of a particular embodiment as “Ac”.

“Alkyl” refers to a hydrocarbon group containing one or more carbon atoms, where multiple carbon atoms if present are joined by single bonds. The alkyl hydrocarbon group may be straight-chain or contain one or more branches or cyclic groups.

“Hydroxyalkyl” refers to an alkyl group wherein one or more hydrogen atoms of the hydrocarbon group are substituted with one or more hydroxy radicals, such as hydroxymethyl, hydroxyethyl, hydroxypropyl, hydroxybutyl, hydroxypentyl, hydroxyhexyl and the like.

“Substituted alkyl” refers to an alkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present. The presence of —(CH₂)₀₋₂₀—COOH results in the production of an alkyl acid. Non-limiting examples of alkyl acids containing, or consisting of, —(CH₂)₀₋₂₀—COOH include 2-norbornane acetic acid, tert-butyric acid, 3-cyclopentyl propionic acid, and the like.

The term “halo” encompasses fluoro, chloro, bromo and iodo.

“Heteroalkyl” refers to an alkyl wherein one of more of the carbon atoms in the hydrocarbon group is replaced with one or more of the following groups: amino, amido, —O—, —S— or carbonyl. In different embodiments 1 or 2 heteroatoms are present.

“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Alkenyl” refers to a hydrocarbon group made up of two or more carbons where one or more carbon-carbon double bonds are present. The alkenyl hydrocarbon group may be straight-chain or contain one or more branches or cyclic groups.

“Substituted alkenyl” refers to an alkenyl wherein one or more hydrogens are replaced with one or more substituents selected from the group consisting of halogen (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Aryl” refers to an optionally substituted aromatic group with at least one ring having a conjugated pi-electron system, containing up to three conjugated or fused ring systems. Aryl includes carbocyclic aryl, heterocyclic aryl and biaryl groups. Preferably, the aryl is a 5- or 6-membered ring. Preferred atoms for a heterocyclic aryl are one or more sulfur, oxygen, and/or nitrogen. Non-limiting examples of aryl include phenyl, 1-naphthyl, 2-naphthyl, indole, quinoline, 2-imidazole, 9-anthracene, and the like. Aryl substituents are selected from the group consisting of —C₁₋₂₀ alkyl, —C₁₋₂₀ alkoxy, halogen (i.e., fluorine, chlorine, bromine, and iodine), —OH, —CN, —SH, —NH₂, —NO₂, —C₁₋₂₀ alkyl substituted with halogens, —CF₃, —OCF₃, and —(CH₂)₀₋₂₀—COOH. In different embodiments the aryl contains 0, 1, 2, 3, or 4 substituents.

“Alkylaryl” refers to an “alkyl” joined to an “aryl”.

The term “(C₁-C₁₂)hydrocarbon moiety” encompasses alkyl, alkenyl and alkynyl and in the case of alkenyl and alkynyl there is C₂-C₁₂.

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

The pharmaceutically acceptable salts of the compounds of the invention which contain a basic center are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically acceptable metal salts, in particular non-toxic alkali and alkaline earth metal salts, with bases. Examples include the sodium, potassium, aluminum, calcium, magnesium, zinc and diethanolamine salts (Berge, S. M. et al., J. Pharm. Sci., 66:1-19 (1977); Gould, P. L., Int'l J. Pharmaceutics, 33:201-17 (1986); and Bighley, L. D. et al., Encyclo. Pharma. Tech., Marcel Dekker Inc, New York, 13:453-97 (1996).

The pharmaceutically acceptable solvates of the compounds of the invention include the hydrates thereof. Also included within the scope of the invention and various salts of the invention are polymorphs thereof. Hereinafter, compounds their pharmaceutically acceptable salts, their solvates or polymorphs, defined in any aspect of the invention (except intermediate compounds in chemical processes) are referred to as “compounds of the invention”.

### EXAMPLES

### In Vitro Studies

Compounds of the present invention can be and were tested for activity as ligands of one or more of the melanocortin receptors according to the following procedures. One skilled in the art would know that procedures similar to those described herein may be used to assay the binding activities of the compounds of the invention to melanocortin receptor molecules.

### Radioligand Binding Assays

Cellular membranes used for the in vitro receptor binding assays were obtained from transgenic CHO-K1 cells stably expressing hMC-R receptor subtypes 1, 3, 4 or 5. The CHO-K1 cells expressing the desired hMC-R receptor type were sonicated (Branson® setting 7, approximately 30 sec) in ice-cold 50 mM Tris-HCl at pH 7.4 and then centrifuged at 39,000 g for 10 minutes at approximately 4° C. The pellets were resuspended in the same buffer and centrifuged at 50,000 g for 10 minutes at approximately 4° C. The washed pellets containing the cellular membranes were stored at approximately −80° C.

Competitive inhibition of [¹²⁵I](Tyr²)-(Nle⁴-D-Phe⁷)α-MSH ([¹²⁵I]-NDP-α-MSH, Amersham Biosciences®) binding was carried out in polypropylene 96 well plates. Cell membranes (1-10 μg protein/well) prepared as described above were incubated in 50 mM Tris-HCl at pH 7.4 containing 0.2% bovine serum albumin (BSA), 5 mM MgCl₂, 1 mM CaCl₂ and 0.1 mg/mL bacitracin, with increasing concentrations of the test compound and 0.1-0.3 nM [²⁵I]-NDP-α-MSH for approximately 90-120 minutes at approximately 37° C. Bound [¹²⁵I]-NDP-α-MSH ligand was separated from free [¹²⁵I]-NDP-α-MSH by filtration through GF/C glass fiber filter plates (Unifilter®; Packard) presoaked with 0.1% (w/v) polyethylenimine (PEI), using a Packard Filtermate® harvester. Filters were washed three times with 50 mM Tris-HCl at pH 7.4 at a temperature of approximately 0-4° C. and then assayed for radioactivity using a Packard Topcount® scintillation counter. Binding data were analyzed by computer-assisted non-linear regression analysis (XL fit; IDBS). A selection of the preferred embodiments was tested using the above-discussed assay and the binding constants (Ki in nM) are reported in Tables 5, 6, 7 and 8.

TABLE 5
Radioligand Binding Assay Data for Selected Compounds

| | | | | | |
|---|---|---|---|---|---|
| | | | | | Ki |
| Compound (according to | Ki | Ki | Ki | Ki | hMC1-R/ |
| Formula I) | hMC1-R | hMC3-R | KMC4-R | hMC5-R | MC4-R |

A

| | | | | | |
|---|---|---|---|---|---|
| Ac-Arg-c(Cys-D-Ala-His-D- | 3.87 | 10.1 | 2.09 | 430 | 1.9 |
| Phe-Arg-Trp-Cys)-NH₂ | | | | | |
| Ac-D-Arg-c(Cys-D-Ala-His- | 4.01 | 12.1 | 1.76 | 352 | 2.3 |
| D-Phe-Arg-Trp-Cys)-NH₂ | | | | | |
| Ac-D-Arg-c(Cys-D-Ala-His- | 8.29 | 13.3 | 2.78 | 816 | 3.0 |
| D-Phe-Arg-Trp-Pen)-NH₂ | | | | | |
| Ac-D-Arg-c(Cys-His-D-Phe- | 3.93 | 172 | 11.0 | 538 | 0.36 |
| Arg-Trp-Gaba-Pen)-NH₂ | | | | | |
| Ac-Arg-c(Cys-His-D-Phe- | 1.81 | 20.5 | 4.57 | 502 | 0.4 |
| Arg-Trp-Gaba-Pen)-NH₂ | | | | | |
| Ac-Arg-c(Cys-D-Ala-His-D- | 9.67 | 22.0 | 4.2 | 1900 | 2.3 |
| Phe-Arg-Trp-Pen)-NH₂ | | | | | |
| Ac-D-Arg-c(Asp-His-D- | 0.79 | 45.5 | 1.21 | 493 | 0.6 |
| Phe-Arg-Trp-Ala-Lys)-NH₂ | | | | | |
| Ac-Arg-c(Asp-His-D-Phe- | 0.68 | 20.7 | 1.01 | 783 | 0.7 |
| Arg-Trp-Ala-Lys)-NH₂ | | | | | |

B

| | | | | | |
|---|---|---|---|---|---|
| Ac-Nle-c(Cys-D-Ala-His-D- | 114 | 63.9 | 3.07 | 1657 | 37.1 |
| 2-Nal-Arg-1-Nal-Cys)-NH₂ | | | | | |
| Ac-Nle-c(Cys-D-Ala-His-D- | 11 | 26 | 7.6 | 1800 | 1.4 |
| Phe-Arg-Trp-Cys)-NH₂ | | | | | |
| D-Phe-c(Cys-His-D-(Et)Tyr- | 0.05 | 9.3 | 1.1 | 2.9 | 0.0 |
| Arg-Trp-β-Ala-D-Cys)-Thr-NH₂ | | | | | |
| Nle-c(Cys-His-D-Phe-Arg- | 0.07 | 4.1 | 0.85 | 8.8 | 0.1 |
| Trp-Apn-Cys)-NH₂ | | | | | |
| Ac-Nle-c(Cys-His-D-Phe- | 0.12 | 10 | 0.43 | 0.42 | 0.3 |
| Arg-Trp-Gaba-Pen)-NH₂ | | | | | |
| Nle-c(Cys-His-D-Phe-Arg- | 0.05 | 1.3 | 0.47 | 0.2 | 0.1 |
| Trp-Gaba-Cys)-NH₂ | | | | | |
| Ac-Nle-c(Asp-His-D-Phe- | 0.0996 | 9318 | 0.617 | 10.9 | 0.16 |
| Arg-Trp-β-Ala-Lys)-NH₂ | | | | | |
| Ac-Nle-c(Cys-His-D-Phe- | .0132 | 16.1 | 1.23 | 0.359 | 0.11 |
| Arg-Trp-Ahx-Cys)-NH₂ | | | | | |
| D-Phe-c(Cys-His-D-Phe- | 0.207 | 43.2 | 2.58 | 344 | 0.08 |
| Arg-Trp-β-Ala-D-Cys)-Thr-NH₂ | | | | | |
| D-Phe-c(Cys-His-D-Phe- | 0.420 | 106 | 4.75 | 1260 | 0.09 |
| Arg-Trp-Gaba-D-Cys)-Thr-NH₂ | | | | | |
| Ac-Nle-c(Cys-His-D-Phe- | 0.0951 | 9.33 | 0.894 | 13.4 | 0.11 |
| Arg-Trp-Apn-Cys)-NH₂ | | | | | |
| Ac-Nle-c(Asp-His-D-Phe- | 0.999 | 300 | 11.1 | 431 | 0.09 |
| Arg-Trp-Apn-Lys)-NH₂ | | | | | |
| Ac-Cha-c(Asp-His-D-Phe- | 0.106 | 11.8 | 1.49 | 110 | 0.07 |
| Arg-Trp-Gaba-Lys)-NH₂ | | | | | |
| Ac-Nle-c(Asp-His-D-Phe- | 0.0506 | 9.89 | 1.04 | 16.3 | 0.05 |
| Arg-Trp-Gaba-Lys)-NH₂ | | | | | |
| Ac-Chg-c(Asp-His-D-Phe- | 0.884 | 223 | 22.5 | 609 | 0.04 |
| Arg-Trp-Gaba-Lys)-NH₂ | | | | | |
| Ac-hCha-c(Asp-His-D-Phe- | 0.721 | 93.5 | 56.0 | 747 | 0.01 |
| Arg-Trp-Gaba-Lys)-NH₂ | | | | | |
| Ac-D-Chg-c(Asp-His-D- | 0.227 | 14.5 | 2.99 | 164 | 0.08 |
| Phe-Arg-Trp-Gaba-Lys)-NH₂ | | | | | |
| Ac-hPhe-c(Asp-His-D-Phe- | 0.277 | 25.2 | 3.37 | 203 | 0.08 |
| Arg-Trp-Gaba-Lys)-NH₂ | | | | | |
| Ac-Nle-c(Cys-His-D-Phe- | 0.323 | 14.1 | 1.96 | 24.0 | 0.16 |
| Arg-D-Trp-β-Ala-Cys)-NH₂ | | | | | |
| Ac-Nle-c(Pen-D-Ala-His-D- | 34.1 | 118 | 17.0 | 5560 | 2.01 |
| Phe-Arg-Trp-Cys)-NH₂ | | | | | |
| Ac-Nle-c(Cys-D-Ala-His-D- | 29.1 | 22.8 | 3.84 | 2550 | 7.58 |
| Phe-Arg-Trp-Pen)-NH₂ | | | | | |
| D-Phe-c(Cys-His-D-Phe- | 0.442 | 123 | 10.3 | 521 | 0.04 |
| hArg-Trp-β-Ala-D-Cys)- | | | | | |
| Thr-NH₂ | | | | | |
| D-Phe-c(Cys-His-D-Phe- | 5.80 | 3370 | 583 | 1130 | 0.01 |
| Arg-Bip-β-Ala-D-Cys)-Thr-NH₂ | | | | | |
| D-Phe-c(Cys-His-D-(Et)Tyr- | 0.0567 | 31.4 | 14.7 | 9.27 | 0 |
| hArg-Trp-β-Ala-D-Cys)- | | | | | |
| Thr-NH₂ | | | | | |
| D-Phe-c(Cys-His-D-Phe- | 1.68 | 1260 | 172 | 1220 | 0.01 |
| hArg-Bip-β-Ala-D-Cys)- | | | | | |
| Thr-NH₂ | | | | | |
| D-Phe-c(Cys-His-D-(Et)Tyr- | 0.128 | 85.6 | 36.9 | 38.0 | 0 |
| hArg-Bip-β-Ala-D-Cys)- | | | | | |
| Thr-NH₂ | | | | | |
| Ac-Nle-c(Cys-D-Ala-His-D- | 0.352 | 149 | 3.01 | 339 | 0.12 |
| Phe-Arg-Trp-Gly-Cys)-NH₂ | | | | | |
| Ac-Nle-c(Cys-D-Ala-His-D- | 3.93 | 876 | 48.0 | 4940 | 0.08 |
| Phe-Arg-Trp-D-Ala-Cys)-NH₂ | | | | | |
| Ac-Nle-c(Cys-D-Ala-His-D- | 0.995 | 287 | 4.80 | 766 | 0.21 |
| Phe-Arg-Trp-β-Ala-Cys)-NH₂ | | | | | |
| Ac-Nle-c(Cys-D-Ala-His-D- | 0.848 | 184 | 3.76 | 956 | 0.23 |
| Phe-Arg-Trp-Gaba-Cys)-NH₂ | | | | | |
| Ac-Nle-c(Cys-D-Ala-His-D- | 1.10 | 228 | 7.58 | 859 | 0.15 |
| Phe-Arg-Trp-Apn-Cys)-NH₂ | | | | | |
| Ac-Nle-c(Asp-D-Ala-His-D- | 0.659 | 98.9 | 2.55 | 4.19 | 0.26 |
| Phe-Arg-Trp-Lys)-NH₂ | | | | | |
| Ac-Nle-c(Asp-D-Ala-His-D- | 4.12 | 445 | 50.6 | 4300 | 0.08 |
| Phe-Arg-Bal-Lys)-NH₂ | | | | | |
| Ac-c(Cys-Glu-His-D-Phe- | 111 | 1710 | 47.7 | 694 | 2.33 |
| Arg-Trp-Ala-Cys)-NH₂ | | | | | |
| Ac-c(Cys-Glu-His-D-Phe- | 262 | 2500 | 96.4 | 1460 | 2.72 |
| Arg-2-Nal-Ala-Cys)-NH₂ | | | | | |
| Ac-c(Cys-D-Ala-His-D-Phe- | 199 | 5990 | 96.7 | >10000 | 2.06 |
| Arg-Trp-Ala-Cys)-NH₂ | | | | | |
| Ac-c(Cys-D-Ala-His-D-Phe- | 132 | 4560 | 40.7 | 8810 | 3.24 |
| Arg-2-Nal-Ala-Cys)-NH₂ | | | | | |
| Ac-Nle-c(Cys-D-Ala-His-D- | 9.12 | 1130 | 22.1 | 2860 | 0.41 |
| Phe-Arg-Trp-Ala-Cys)-NH₂ | | | | | |
| Ac-Nle-c(Cys-D-Ala-His-D- | 1.00 | 227 | 5.55 | 496 | 0.18 |
| Phe-Arg-Trp-β-Ala-Cys)-NH₂ | | | | | |
| Ac-Nle-c(Cys-D-Ala-His-D- | 0.536 | 169 | 3.12 | 358 | 0.17 |
| Phe-Arg-Trp-Gaba-Cys)-NH₂ | | | | | |
| Ac-Nle-c(Cys-D-Ala-His-D- | 32.1 | 330 | 17.4 | 165 | 1.84 |
| Phe-Arg-Trp-Pen)-OH | | | | | |
| Ac-Nle-c(Cys-D-Abu-His- | 10.6 | 41.1 | 7.69 | 54.9 | 1.38 |
| D-Phe-Arg-Trp-Cys)-NH₂ | | | | | |
| Ac-Nle-c(Cys-D-Val-His-D- | 13.0 | 104 | 10.1 | 40 | 1.29 |
| Phe-Arg-Trp-Cys)-NH₂ | | | | | |
| Ac-Nle-c(Cys-D-Ile-His-D- | 4.28 | 38.5 | 9.0 | 12.5 | 0.48 |
| Phe-Arg-Trp-Cys)-NH₂ | | | | | |
| Ac-Nle-c(Cys-D-Leu-His-D- | 1.60 | 6.82 | 4.13 | 5.57 | 0.39 |
| Phe-Arg-Trp-Cys)-NH₂ | | | | | |
| Ac-Nle-c(Cys-D-Tle-His-D- | 12.0 | 85.8 | 11.2 | 40 | 1.07 |
| Phe-Arg-Trp-Cys)-NH₂ | | | | | |
| Ac-Nle-c(Cys-D-Cha-His-D- | 0.353 | 2.08 | 1.41 | 0.857 | 0.25 |
| Phe-Arg-Trp-Cys)-NH₂ | | | | | |
| Ac-Nle-c(Pen-His-D-Phe- | 0.537 | 86.1 | 5.89 | 2.56 | 0.09 |
| Arg-Trp-Gaba-Cys)-NH₂ | | | | | |
| Ac-Nle-c(Pen-His-D-Phe- | 0.744 | 178 | 3.51 | 2.69 | 0.21 |
| Arg-Trp-Gaba-Pen)-NH₂ | | | | | |
| Ac-Leu-c(Cys-His-D-Phe- | 0.216 | 17.4 | 0.995 | 0.486 | 0.22 |
| Arg-Trp-Gaba-Cys)-NH₂ | | | | | |
| Ac-Cha-c(Cys-His-D-Phe- | 0.107 | 9.11 | 0.884 | 0.354 | 0.12 |
| Arg-Trp-Gaba-Cys)-NH₂ | | | | | |
| Ac-Ile-c(Cys-His-D-Phe- | 0.148 | 13.9 | 1.06 | 0.423 | 0.14 |
| Arg-Trp-Gaba-Cys)-NH₂ | | | | | |
| Ac-Phe-c(Cys-His-D-Phe- | 0.254 | 18.5 | 2.13 | 0.714 | 0.12 |
| Arg-Trp-Gaba-Cys)-NH₂ | | | | | |
| Ac-Val-c(Cys-His-D-Phe- | 0.256 | 29.9 | 1.98 | 0.864 | 0.13 |
| Arg-Trp-Gaba-Cys)-NH₂ | | | | | |
| Ac-2-Nal-c(Cys-His-D-Phe- | 0.560 | 39.2 | 2.94 | 2.73 | 0.19 |
| Arg-Trp-Gaba-Cys)-NH₂ | | | | | |
| Phe-c(Cys-His-D-Phe-Arg- | 0.186 | 15.2 | 4.93 | 0.537 | 0.04 |
| Trp-Gaba-Cys)-NH₂ | | | | | |
| Ac-Nle-c(Cys-3-Pal-D-Phe- | 21.1 | 151 | 10.4 | 92.6 | 2.03 |
| Arg-Trp-Gaba-Cys)-NH₂ | | | | | |
| Ac-Nle-c(Cys-D-Ala-His-D- | 30.7 | 152 | 15.6 | 114 | 1.97 |
| Phe-Arg-Trp-Cys)-OH | | | | | |
| Ac-Nle-c(Cys-His-Phe-Arg- | 5.20 | 150 | 138 | 20.3 | 0.04 |
| D-Trp-Gaba-Cys)-NH₂ | | | | | |
| Ac-Nle-c(Asp-D-Ala-His-D- | 4.89 | 290 | 21.3 | 11.1 | 0.23 |
| Phe-Arg-Bal-Ala-Lys)-NH₂ | | | | | |
| Ac-Nle-c(Cys-D-Ala-His-D- | 25.5 | 3.82 | 7.61 | 102 | 3.35 |
| 2-Nal-Arg-Trp-Cys)-NH₂ | | | | | |
| Ac-Nle-c(Cys-D-Ala-His-D- | 32.5 | 5.85 | 2.53 | 94.6 | 12.85 |
| 2-Nal-Arg-2-Nal-Cys)-NH₂ | | | | | |
| Ac-Nle-c(Cys-D-Ala-His-D- | 22.2 | 12.7 | 16.6 | 125 | 1.34 |
| 2-Nal-Arg-Bal-Cys)-NH₂ | | | | | |
| Ac-Nle-c(Asp-His-D-2-Nal- | 1.17 | 1.56 | 0.277 | 3.24 | 4.22 |
| Arg-Trp-Ala-Lys)-NH₂ | | | | | |
| Ac-Nle-c(Asp-His-D-2-Nal- | 0.648 | 2.78 | 0.329 | 1.4 | 1.97 |
| Arg-Trp-β-Ala-Lys)-NH₂ | | | | | |
| Ac-Nle-c(Cys-His-D-2-Nal- | 0.393 | 1.86 | 0.375 | 1.11 | 1.05 |
| Arg-Trp-Gaba-Cys)-NH₂ | | | | | |
| Ac-Nle-c(Cys-His-D-2-Nal- | 0.333 | 2.91 | 0.998 | 0.366 | 0.33 |
| Arg-Trp-Ahx-Cys)-NH₂ | | | | | |
| Ac-hPhe-c(Asp-His-D-2- | 0.461 | 2.45 | 0.931 | 1.37 | 0.50 |
| Nal-Arg-Trp-Gaba-Lys)-NH₂ | | | | | |
| Ac-Cha-c(Asp-His-D-2-Nal- | 0.576 | 3.98 | 2.82 | 3.91 | 0.20 |
| Arg-Trp-Gaba-Lys)-NH₂ | | | | | |

C

| | | | | | |
|---|---|---|---|---|---|
| Ac-Arg-c(Cys-D-Ala-His-D- | 17.9 | 1.68 | 0.256 | 23.4 | 69.9 |
| 2-Nal-Arg-Trp-Cys)-NH₂ | | | | | |

TABLE 6
Radioligand Binding Assay Data for Selected Compounds

| | | | |
|---|---|---|---|
| Ki | Ki | Ki | Ki |
| hMC1-R | hMC3-R | hMC4-R | hMC5-R |

Table 6A

| | | | | |
|---|---|---|---|---|
| Compound (according to Formula III) | | | | |
| Ac-c(Cys-Glu-His-D-2-Nal-Arg-Trp-Ala- | 49.9 | 9.00 | 0.569 | 218 |
| Cys)-Pro-Pro-Lys-Asp-NH₂ | | | | |
| Tyr-Gly-Arg-Lys-Lys-Arg-Arg-Gln-Arg-Arg- | 11.9 | 38.1 | 5.70 | 11.8 |
| Arg-Nle-c(Asp-His-D-2-Nal-Arg-Trp-Lys)- | | | | |
| NH₂ | | | | |
| Tyr-Gly-Arg-Lys-Lys-Arg-Arg-Gln-Arg-Arg- | 3.46 | 16.6 | 6.65 | 4.88 |
| Arg-Doc-Nle-c(Asp-His-D-2-Nal-Arg-Trp- | | | | |
| Lys)-NH₂ | | | | |
| Nle-c(Asp-His-D-2-Nal-Arg-Trp-Lys)-β-Ala- | 0.614 | 5.09 | 2.31 | 3.23 |
| Tyr-Gly-Arg-Lys-Lys-Arg-Arg-Gln-Arg-Arg- | | | | |
| Arg-NH₂ | | | | |
| Ac-Nle-c(Asp-His-D-2-Nal-Arg-Trp-Lys)-β- | 1.56 | 14.1 | 5.17 | 7.12 |
| Ala-Tyr-Gly-Arg-Lys-Lys-Arg-Arg-Gln-Arg- | | | | |
| Arg-Arg-NH₂ | | | | |
| H-Nle-c(Asp-His-D-2-Nal-Arg-Trp-Lys)-Doc- | 1.10 | 1.58 | 6.00 | 0.629 |
| Doc-Tyr-Gly-Arg-Lys-Lys-Arg-Arg-Gln-Arg- | | | | |
| Arg-Arg-NH₂ | | | | |
| Ac-Nle-c(Asp-His-D-2-Nal-Arg-Trp-Lys)- | 0.0868 | 0.751 | 0.0944 | 0.147 |
| Pro-Pro-Lys-Asp-Tyr-Gly-Arg-Lys-Lys-Arg- | | | | |
| Arg-Gln-Arg-Arg-Arg-NH₂ | | | | |
| Ac-c(Cys-Glu-His-D-2-Nal-Arg-Trp-Gly- | 1.66 | 4.80 | 0.250 | 9.62 |
| Cys)-Pro-Pro-Lys-Asp-Tyr-Gly-Arg-Lys-Lys- | | | | |
| Arg-Arg-Gln-Arg-Arg-Arg-NH₂ | | | | |
| Ac-Nle-c(Asp-His-D-2-Nal-Arg-Trp-Lys)-β- | 0.0452 | 0.298 | 0.169 | 0.386 |
| Ala-β-Ala-Tyr-Gly-Arg-Lys-Lys-Arg-Arg- | | | | |
| Gln-Arg-Arg-Arg-NH₂ | | | | |
| Ac-Nle-c(Asp-His-D-2-Nal-Arg-Trp-Lys)- | 0.0808 | 0.396 | 0.0747 | 0.311 |
| Pro-Pro-Lys-Asp-Doc-Tyr-Gly-Arg-Lys-Lys- | | | | |
| Arg-Arg-Gln-Arg-Arg-Arg-NH₂ | | | | |
| Ac-c(Cys-Glu-His-D-2-Nal-Arg-Trp-Gly- | 4.41 | 4.23 | 0.455 | 12.9 |
| Cys)-Pro-Pro-Lys-Asp-Doc-Tyr-Gly-Arg-Lys- | | | | |
| Lys-Arg-Arg-Gln-Arg-Arg-Arg-NH₂ | | | | |
| Ac-c(Cys-Glu-His-D-2-Nal-Arg-Trp-Ala- | 1.25 | 0.661 | 0.292 | 5.94 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Trp-Ala- | 1.89 | 0.546 | 0.166 | 6.06 |
| Cys)-Pro-Pro-Lys-Asp-Doc-Tyr-Gly-Arg-Lys- | | | | |
| Lys-Arg-Arg-Gln-Arg-Arg-Arg-NH₂ | | | | |
| Ac-c(Cys-Glu-His-D-2-Nal-Arg-2-Nal-Ala- | 87.8 | 9.08 | 1.20 | 359 |
| Cys)-Pro-Pro-Lys-Asp-NH₂ | | | | |
| Ac-c(Cys-Glu-His-D-2-Nal-Arg-1-Nal-Ala- | 124 | 17.8 | 1.11 | 348 |
| Cys)-Pro-Pro-Lys-Asp-NH₂ | | | | |
| Ac-c(Cys-Glu-His-D-2-Nal-Arg-Bal-Ala-Cys)- | 163 | 23.0 | 0.586 | 844 |
| Pro-Pro-Lys-Asp-NH₂ | | | | |
| Ac-Nle-c(Asp-His-D-2-Nal-Arg-Trp-Lys)- | 0.144 | 0.352 | 0.0845 | 0.415 |
| Doc-Doc-Tyr-Gly-Arg-Lys-Lys-Arg-Arg-Gln- | | | | |
| Arg-Arg-Arg-NH₂ | | | | |
| Ac-c(Cys-Glu-His-D-2-Nal-Arg-2-Nal-Ala- | 1.74 | 0.590 | 0.170 | 4.38 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Bal-Ala-Cys)- | 3.86 | 4.97 | 0.192 | 38.3 |
| Pro-Pro-Lys-Asp-β-Ala-Arg-Arg-Arg-Arg- | | | | |
| Arg-Gln-Arg-Arg-Arg-NH₂ | | | | |
| Ac-c(Cys-Glu-His-D-2-Nal-Arg-Bal-Ala-Cys)- | 12.8 | 15.9 | 0.950 | 165 |
| Pro-Pro-Lys-Asp-β-Ala-Gly-Arg-Arg-Arg- | | | | |
| Arg-Arg-Gln-Arg-Arg-Arg-NH₂ | | | | |
| Ac-c(Cys-Glu-His-D-2-Nal-Arg-Bal-Ala-Cys)- | 3.07 | 4.05 | 0.498 | 31.1 |
| Pro-Pro-Lys-Asp-β-Ala-Tyr-Gly-Arg-Arg- | | | | |
| Arg-Arg-Arg-Gln-Arg-Arg-Arg-NH₂ | | | | |
| Ac-c(Cys-Glu-His-D-2-Nal-Arg-Trp-Ala- | 0.792 | 0.570 | 0.162 | 4.18 |
| Cys)-Pro-Pro-Lys-Asp-βAla-Tyr-Gly-Arg- | | | | |
| Lys-Lys-Arg-Gln-Arg-Arg-Arg-Arg-NH₂ | | | | |
| Ac-c(Cys-Glu-His-D-2-Nal-Arg-Trp-Ala- | 0.726 | 0.474 | 0.209 | 5.12 |
| Cys)-Pro-Pro-Lys-Asp-β-Ala-Tyr-Gly-Arg- | | | | |
| Lys-Lys-Gln-Arg-Arg-Arg-Arg-Arg-NH₂ | | | | |
| Ac-c(Cys-Glu-His-D-2-Nal-Arg-Trp-Ala- | 0.857 | 0.580 | 0.209 | 4.42 |
| Cys)-Pro-Pro-Lys-Asp-β-Ala-Tyr-Gly-Arg- | | | | |
| Lys-Gln-Lys-Arg-Arg-Arg-Arg-Arg-NH₂ | | | | |
| Ac-c(Cys-Glu-His-D-2-Nal-Arg-Trp-Ala- | 0.813 | 0.675 | 0.269 | 4.20 |
| Cys)-Pro-Pro-Lys-Asp-β-Ala-Tyr-Gly-Arg- | | | | |
| Lys-Lys-Arg-Arg-Arg-Arg-Gln-Arg-NH₂ | | | | |
| Ac-c(Cys-Glu-His-D-2-Nal-Arg-Bal-Ala-Cys)- | 7.84 | 10.2 | 0.783 | 91.8 |
| Pro-Pro-Lys-Asp-β-Ala-Tyr-Aib-Arg-Lys- | | | | |
| Lys-Arg-Arg-Gln-Arg-Arg-Arg-NH₂ | | | | |
| Ac-c(Cys-Glu-His-D-2-Nal-Arg-1-Nal-Ala- | 2.93 | 9.07 | 0.293 | 59.0 |
| Cys)-Pro-Pro-Arg-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-1-Nal-Ala- | 2.42 | 6.56 | 0.238 | 41.7 |
| Cys)-Pro-Pro-Lys-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-1-Nal-Ala- | 6.66 | 19.3 | 0.819 | 88.8 |
| Cys)-Pro-Pro-Lys-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-2-Nal-Ala- | 2.63 | 2.09 | 0.0737 | 11.6 |
| Cys)-Pro-Pro-Arg-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-2-Nal-Ala- | 2.48 | 1.21 | 0.209 | 9.17 |
| Cys)-Pro-Pro-Lys-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-2-Nal-Ala- | 3.65 | 2.26 | 0.261 | 12.1 |
| Cys)-Pro-Pro-Lys-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-Bal-Ala-Cys)- | 7.32 | 11.0 | 0.659 | 78.0 |
| Pro-Pro-Arg-Asp-β-Ala-Arg-Arg-Arg-Arg- | | | | |
| Arg-Arg-Gln-Arg-Arg-Arg-NH₂ | | | | |
| Ac-c(Cys-Glu-His-D-2-Nal-Arg-Bal-Ala-Cys)- | 4.11 | 7.26 | 0.302 | 48.3 |
| Pro-Pro-Arg-Asp-β-Ala-Arg-Arg-Arg-Arg- | | | | |
| Arg-Gln-Arg-Arg-Arg-NH₂ | | | | |
| Ac-c(Cys-Glu-His-D-2-Nal-Arg-Bal-Ala-Cys)- | 6.77 | 14.3 | 0.781 | 84.0 |
| Pro-Pro-Lys-Asp-β-Ala-Arg-Arg-Arg-Arg- | | | | |
| Arg-Arg-Gln-Arg-Arg-Arg-NH₂ | | | | |
| Ac-c(Cys-Glu-His-D-2-Nal-Arg-Trp-Ala- | 3.04 | 3.22 | 0.230 | 3.85 |
| Cys)-Pro-Pro-Lys-Asp-β-Ala-Tyr-Gly-Arg- | | | | |
| Lys-Lys-Arg-Arg-Arg-Gln-Arg-Arg-NH₂ (85) | | | | |
| Ac-c(Cys-Glu-His-D-2-Nal-Arg-Trp-Ala- | 3.24 | 2.66 | 0.208 | 5.96 |
| Cys)-Pro-Pro-Lys-Asp-β-Ala-Tyr-Gly-Arg- | | | | |
| Gln-Lys-Lys-Arg-Arg-Arg-Arg-Arg-NH₂ | | | | |
| Ac-c(Cys-Glu-His-D-2-Nal-Arg-Trp-Ala- | 1.58 | 1.43 | 0.275 | 2.97 |
| Cys)-Pro-Pro-Lys-Asp-β-Ala-Tyr-Gly-Arg- | | | | |
| Lys-Lys-Arg-Arg-Arg-Arg-Arg-Gln-NH₂ | | | | |
| Ac-c(Cys-Glu-His-D-2-Nal-Arg-1-Nal-Ala- | 4.59 | 6.28 | 0.588 | 22.6 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Bal-Ala-Cys)- | 6.46 | 5.22 | 0.380 | 15.3 |
| Pro-Pro-Lys-Asp-β-Ala-Tyr-Gly-Arg-Lys- | | | | |
| Lys-Arg-Arg-Gln-Arg-Arg-Arg-NH₂ | | | | |
| Ac-c(Cys-Glu-His-D-2-Nal-Arg-1-Nal-Ala- | 4.62 | 5.68 | 0.505 | 45.3 |
| Cys)-Pro-Pro-Lys-Asp-β-Ala-Arg-Arg-Lys- | | | | |
| Arg-Arg-Gln-Arg-Arg-Arg-NH₂ | | | | |
| Ac-c(Cys-Glu-His-D-2-Nal-Arg-1-Nal-Ala- | 2.12 | 3.99 | 0.352 | 27.5 |
| Cys)-Pro-Pro-Lys-Asp-β-Ala-Arg-Lys-Arg- | | | | |
| Arg-Arg-Gln-Arg-Arg-Arg-NH₂ | | | | |
| Ac-c(Cys-Glu-His-D-2-Nal-Arg-2-Nal-Ala- | 3.41 | 0.975 | 0.549 | 11.3 |
| Cys)-Pro-Pro-Lys-Asp-β-Ala-Arg-Arg-Lys- | | | | |
| Arg-Arg-Gln-Arg-Arg-Arg-NH₂ | | | | |
| Ac-c(Cys-Glu-His-D-2-Nal-Arg-2-Nal-Ala- | 4.18 | 1.12 | 0.223 | 15.3 |
| Cys)-Pro-Pro-Lys-Asp-β-Ala-Tyr-Gly-Arg- | | | | |
| Arg-Lys-Arg-Arg-Gln-Arg-Arg-Arg-NH₂ | | | | |
| Ac-c(Cys-Glu-His-D-2-Nal-Arg-2-Nal-Ala- | 2.71 | 0.732 | 0.202 | 5.53 |
| Cys)-Pro-Pro-Lys-Asp-β-Ala-Gly-Arg-Arg- | | | | |
| Lys-Arg-Arg-Gln-Arg-Arg-Arg-NH₂ | | | | |
| Ac-c(Cys-Glu-His-D-2-Nal-Arg-2-Nal-Ala- | 5.66 | 1.40 | 0.446 | 6.23 |
| Cys)-Pro-Pro-Lys-Asp-β-Ala-Gly-Arg-Lys- | | | | |
| Arg-Arg-Arg-Gln-Arg-Arg-Arg-NH₂ | | | | |
| Ac-Nle-c(Cys-D-Ala-His-D-Phe-Arg-Trp- | 0.211 | 0.665 | 0.635 | 118 |
| Cys)-(Doc)2-Tyr-Gly-Arg-Lys-Lys-Arg-Arg- | | | | |
| Gln-Arg-Arg-Arg-NH₂ | | | | |
| Ac-Nle-c(Cys-D-Ala-His-D-Phe-Arg-Trp- | 0.351 | 0.891 | 0.503 | 102 |
| Cys)-β-Ala-Tyr-Gly-Arg-Lys-Lys-Arg-Gln- | | | | |
| Arg-Arg-Arg-Arg-NH₂ | | | | |
| Ac-Nle-c(Cys-D-Ala-His-D-Phe-Arg-Trp- | 0.209 | 0.699 | 0.596 | 137 |
| Cys)-Doc-Tyr-Gly-Arg-Lys-Lys-Arg-Arg- | | | | |
| Gln-Arg-Arg-Arg-NH₂ | | | | |
| Ac-Nle-c(Cys-D-Ala-His-D-Phe-Arg-Trp- | 0.439 | 1.52 | 0.476 | 115 |
| Cys)-β-Ala-Gly-Arg-Arg-Arg-Arg-Arg-Gln- | | | | |
| Arg-Arg-Arg-NH₂ | | | | |
| Ac-Nle-c(Cys-D-Ala-His-D-Phe-Arg-Trp- | 0.821 | 2.50 | 0.700 | 148 |
| Cys)-β-Ala-Arg-Arg-Arg-Arg-Arg-Gln-Arg- | | | | |
| Arg-Arg-NH₂ | | | | |
| Ac-Nle-c(Cys-D-Ala-His-D-Phe-Arg-Trp- | 0.406 | 1.11 | 0.602 | 131 |
| Cys)-β-Ala-Tyr-Gly-Arg-Arg-Arg-Arg-Arg- | | | | |
| Gln-Arg-Arg-Arg-NH₂ | | | | |
| Ac-Nle-c(Cys-D-Ala-His-D-Phe-Arg-Trp- | 1.27 | 4.63 | 1.51 | 220 |
| Cys)-β-Ala-Gly-Arg-Arg-Arg-Arg-Arg-Gln- | | | | |
| Arg-Arg-Arg-Arg-NH₂ | | | | |

Table 6B

| | | | | |
|---|---|---|---|---|
| Compound (according to Formula IV) | | | | |
| Ac-c(Cys-Glu-His-D-2-Nal-Arg-2-Nal-β-Ala- | 2058 | 113 | 10.7 | 239 |
| Cys)-Pro-Pro-Lys-Asp-NH₂ | | | | |
| Ac-c(Cys-Glu-His-D-2-Nal-Arg-2-Nal-Aib- | 1818 | 306 | 5.87 | 979 |
| Cys)-Pro-Pro-Lys-Asp-NH₂ | | | | |
| Ac-c(Cys-Glu-His-D-2-Nal-Arg-1-Nal-Ala- | 1.75 | 1.74 | 0.15 | 16.8 |
| Cys)-Pro-Pro-Lys-Asp-β-Ala-Tyr-Gly-Arg- | | | | |
| Arg-Lys-Arg-Arg-Gln-Arg-Arg-Arg-NH₂ | | | | |
| Ac-c(Cys-Glu-His-D-2-Nal-Arg-1-Nal-Ala- | 1.50 | 1.61 | 0.301 | 10.4 |
| Cys)-Pro-Pro-Lys-Asp-β-Ala-Tyr-Gly-Arg- | | | | |
| Lys-Arg-Arg-Arg-Gln-Arg-Arg-Arg-NH₂ | | | | |
| Ac-c(Cys-Glu-His-D-2-Nal-Arg-1-Nal-Ala- | 1.81 | 2.08 | 0.305 | 19.3 |
| Cys)-Pro-Pro-Lys-Asp-β-Ala-Gly-Arg-Arg- | | | | |
| Lys-Arg-Arg-Gln-Arg-Arg-Arg-NH₂ | | | | |
| Ac-c(Cys-Glu-His-D-2-Nal-Arg-1-Nal-Ala- | 2.69 | 2.59 | 0.243 | 19.2 |
| Cys)-Pro-Pro-Lys-Asp-β-Ala-Gly-Arg-Lys- | | | | |
| Arg-Arg-Arg-Gln-Arg-Arg-Arg-NH₂ | | | | |
| Ac-c(Cys-Glu-His-D-2-Nal-Arg-2-Nal-Ala- | 2.25 | 0.62 | 0.303 | 2.77 |
| Cys)-Pro-Pro-Lys-Asp-β-Ala-Arg-Lys-Arg- | | | | |
| Arg-Arg-Gln-Arg-Arg-Arg-NH₂ | | | | |
| Ac-c(Cys-Glu-His-D-2-Nal-Arg-2-Nal-Ala- | 1.49 | 0.604 | 0.865 | 3.13 |
| Cys)-Pro-Pro-Lys-Asp-β-Ala-Tyr-Gly-Arg- | | | | |
| Lys-Arg-Arg-Arg-Gln-Arg-Arg-Arg-NH₂ | | | | |
| Ac-c(Cys-Glu-His-D-2-Nal-Arg-Bal-Ala-Cys)- | 3.28 | 1.95 | 0.575 | 15.5 |
| Pro-Pro-Lys-Asp-β-Ala-Arg-Lys-Arg-Arg- | | | | |
| Arg-Gln-Arg-Arg-Arg-NH₂ | | | | |
| Ac-c(Cys-Glu-His-D-2-Nal-Arg-Bal-Ala-Cys)- | 2.24 | 1.57 | 0.437 | 16.4 |
| Pro-Pro-Lys-Asp-β-Ala-Tyr-Gly-Arg-Arg- | | | | |
| Lys-Arg-Arg-Gln-Arg-Arg-Arg-NH₂ | | | | |
| Ac-c(Cys-Glu-His-D-2-Nal-Arg-Bal-Ala-Cys)- | 2.14 | 1.12 | 0.624 | 11.9 |
| Pro-Pro-Lys-Asp-β-Ala-Tyr-Gly-Arg-Lys- | | | | |
| Arg-Arg-Arg-Gln-Arg-Arg-Arg-NH₂ | | | | |
| Ac-c(Cys-Glu-His-D-2-Nal-Arg-Bal-Ala-Cys)- | 2.50 | 1.59 | 0.573 | 15.7 |
| Pro-Pro-Lys-Asp-β-Ala-Gly-Arg-Arg-Lys- | | | | |
| Arg-Arg-Gln-Arg-Arg-Arg-NH₂ | | | | |
| Ac-c(Cys-Glu-His-D-2-Nal-Arg-Bal-Ala-Cys)- | 3.00 | 1.70 | 0.442 | 15.5 |
| Pro-Pro-Lys-Asp-β-Ala-Arg-Arg-Lys-Arg- | | | | |
| Arg-Gln-Arg-Arg-Arg-NH₂ | | | | |
| Ac-c(Cys-Glu-His-D-2-Nal-Arg-Bal-Ala-Cys)- | 4.29 | 2.15 | 0.425 | 15.5 |
| Pro-Pro-Lys-Asp-β-Ala-Gly-Arg-Lys-Arg- | | | | |
| Arg-Arg-Gln-Arg-Arg-Arg-NH₂ | | | | |
| Ac-Nle-c(Cys-D-Ala-His-D-Phe-Arg-Trp- | 0.410 | 0.837 | 0.246 | 56.3 |
| Cys)-β-Ala-Tyr-Gly-Arg-Arg-Lys-Arg-Arg- | | | | |
| Gln-Arg-Arg-Arg-NH₂ | | | | |
| Ac-Nle-c(Cys-D-Ala-His-D-Phe-Arg-Trp- | 0.572 | 1.07 | 0.210 | 63.6 |
| Cys)-β-Ala-Tyr-Gly-Arg-Lys-Arg-Arg-Arg- | | | | |
| Gln-Arg-Arg-Arg-NH₂ | | | | |
| Ac-Nle-c(Cys-D-Ala-His-D-Phe-Arg-Trp- | 0.475 | 0.800 | 0.196 | 53.8 |
| Cys)-β-Ala-Gly-Arg-Arg-Lys-Arg-Arg-Gln- | | | | |
| Arg-Arg-Arg-NH₂ | | | | |
| Ac-Nle-c(Cys-D-Ala-His-D-Phe-Arg-Trp- | 0.779 | 1.21 | 0.293 | 56.0 |
| Cys)-β-Ala-Gly-Arg-Lys-Arg-Arg-Arg-Gln- | | | | |
| Arg-Arg-Arg-NH₂ | | | | |
| Ac-Nle-c(Cys-D-Ala-His-D-Phe-Arg-Trp- | 0.212 | 1.23 | 0.484 | 58.5 |
| Cys)-β-Ala-Arg-Arg-Lys-Arg-Arg-Gln-Arg- | | | | |
| Arg-Arg-NH₂ | | | | |
| Ac-Nle-c(Cys-D-Ala-His-D-Phe-Arg-Trp- | 0.778 | 1.22 | 0.468 | 47.0 |
| Cys)-β-Ala-Arg-Lys-Arg-Arg-Arg-Gln-Arg- | | | | |
| Arg-Arg-NH₂ | | | | |

TABLE 7

| | | | |
|---|---|---|---|
| Ki | Ki | Ki | Ki |
| hMC1 | hMC3 | hMC4 | hMC5 |

Binding Constants for Formula (V) Examples
Table 7A

| | | | | |
|---|---|---|---|---|
| Formula (V) Compounds | | | | |
| c[Hydantoin(C(O)-(Cys-D-Ala))-His-D-Phe- | 230 | 7590 | 126 | 7020 |
| Arg-Trp-Cys]-NH₂ | | | | |
| c[Hydantoin(C(O)-(Glu-D-Ala))-His-D-Phe- | 72.6 | 1920 | 45.2 | >10000 |
| Arg-Trp-Lys]-NH₂ | | | | |
| c[Hydantoin(C(O)-(Glu-D-Ala))-His-D-Phe- | 60.4 | 2840 | 52.4 | >10000 |
| Arg-Trp-Orn]-NH₂ | | | | |
| c[Hydantoin(C(O)-(Glu-D-Ala))-His-D-Phe- | 28 | 90.5 | 12.7 | 877 |
| Arg-Trp-Dab]-NH₂ | | | | |
| c[Hydantoin(C(O)-(Glu-D-Ala))-His-D-Phe- | 16.4 | 863 | 4.97 | >10000 |
| Arg-Trp-Dap]-NH₂ | | | | |
| c[Hydantoin(C(O)-(Asp-D-Ala))-His-D-Phe- | 37.7 | 576 | 7.81 | 6400 |
| Arg-Trp-Orn]-NH₂ | | | | |
| c[Hydantoin(C(O)-(Asp-D-Ala))-His-D-Phe- | 66.6 | 1820 | 19.9 | >10000 |
| Arg-Trp-Dap]-NH₂ | | | | |
| c[Hydantoin(C(O)-(Asp-His))-D-2-Nal-Arg- | 200 | 68.8 | 6.63 | 142 |
| Trp-Lys]-NH₂ | | | | |
| c[Hydantoin(C(O)-(Asp-Aic))-D-2-Nal-Arg- | 9028 | 2628 | 35.8 | 1156 |
| Trp-Lys]-NH₂ | | | | |
| c[Hydantoin(C(O)-(Asp-A5c))-D-2-Nal-Arg- | 9938 | 2390 | 44.6 | 1103 |
| Trp-Lys]-NH₂ | | | | |
| c[Hydantoin(C(O)-(Asp-A6c))-D-2-Nal-Arg- | 2170 | 1479 | 16.5 | 451 |
| Trp-Lys]-NH₂ | | | | |
| c[Hydantoin(C(O)-(Asp-Apc))-D-2-Nal-Arg- | 1276 | 2756 | 266 | 1096 |
| Trp-Lys]-NH₂ | | | | |
| c[Hydantoin(C(O)-(Asp-A3c))-D-2-Nal-Arg- | 7567 | 1922 | 420 | 2879 |
| Trp-Lys]-NH₂ | | | | |

TABLE 7B - Binding Constants for Formula (VI) Examples

| | | | | |
|---|---|---|---|---|
| Formula (VI) Compounds | | | | |
| Hydantoin(C(O)-(Nle-Gly))-c(Cys-Glu-His-D- | 14.3 | 198 | 5.76 | 67.8 |
| Phe-Arg-Trp-Cys)-NH₂ | | | | |
| Hydantoin(C(O)-(Gly-Gly))-c(Cys-Glu-His-D- | 11.9 | 311 | 5.41 | 73.9 |
| Phe-Arg-Trp-Cys)-NH₂ | | | | |
| Hydantoin(C(O)-(A6c-Nle))-c(Cys-D-Ala-His- | 31.6 | 224 | 19.6 | 2500 |
| D-Phe-Arg-Trp-Cys)-NH₂ | | | | |
| Hydantoin(C(O)-(D-Ala-Nle))-c(Cys-D-Ala- | 16.0 | 63.9 | 8.64 | 1820 |
| His-D-Phe-Arg-Trp-Cys)-NH₂ | | | | |
| Hydantoin(C(O)-(Val-Nle))-c(Cys-D-Ala-His- | 33.7 | 132 | 40.2 | 3210 |
| D-Phe-Arg-Trp-Cys)-NH₂ | | | | |
| Hydantoin(C(O)-(Leu-Nle))-c(Cys-D-Ala-His- | 48.3 | 534 | 74.1 | 3290 |
| D-Phe-Arg-Trp-Cys)-NH₂ | | | | |
| Hydantoin(C(O)-(Cha-Nle))-c(Cys-D-Ala-His- | 40.8 | 870 | 137 | 3560 |
| D-Phe-Arg-Trp-Cys)-NH₂ | | | | |
| Hydantoin(C(O)-(Aib-Nle))-c(Cys-D-Ala-His- | 17.7 | 73.6 | 8.40 | 2120 |
| D-Phe-Arg-Trp-Cys)-NH₂ | | | | |
| Hydantoin(C(O)-(Nle-Gly))-c(Cys-D-Ala-His- | 7.92 | 46.4 | 6.70 | 21.3 |
| D-Phe-Arg-Trp-Cys)-NH₂ | | | | |
| Hydantoin(C(O)-(Gly-Gly))-c(Cys-D-Ala-His- | 20.9 | 69.7 | 8.32 | 50.0 |
| D-Phe-Arg-Trp-Cys)-NH₂ | | | | |
| Hydantoin(C(O)-(Nle-Gly))-c(Cys-D-Ala-His- | 12.9 | 38.5 | 3.53 | 27.1 |
| D-Phe-Arg-Trp-Pen)-NH₂ | | | | |
| Hydantoin(C(O)-(Gly-Gly))-c(Cys-D-Ala-His- | 127 | 811 | 10.4 | 381 |
| D-Phe-Arg-Trp-Pen)-NH₂ | | | | |
| Hydantoin(C(O)-(Ala-Gly))-c(Cys-D-Ala-His- | 13.9 | 38.4 | 5.73 | 18.9 |
| D-Phe-Arg-Trp-Cys)-NH₂ | | | | |
| Hydantoin(C(O)-(D-Ala-Gly))-c(Cys-D-Ala- | 11.7 | 73.1 | 4.28 | 34.7 |
| His-D-Phe-Arg-Trp-Cys)-NH₂ | | | | |
| Hydantoin(C(O)-(Aib-Gly))-c(Cys-D-Ala-His- | 36.8 | 290 | 13.7 | 133 |
| D-Phe-Arg-Trp-Cys)-NH₂ | | | | |
| Hydantoin(C(O)-(Val-Gly))-c(Cys-D-Ala-His- | 15.3 | 160 | 8.66 | 33.4 |
| D-Phe-Arg-Trp-Cys)-NH₂ | | | | |
| Hydantoin(C(O)-(Ile-Gly))-c(Cys-D-Ala-His- | 11.6 | 194 | 11.5 | 28.9 |
| D-Phe-Arg-Trp-Cys)-NH₂ | | | | |
| Hydantoin(C(O)-(Leu-Gly))-c(Cys-D-Ala-His- | 19.3 | 331 | 26.7 | 44.6 |
| D-Phe-Arg-Trp-Cys)-NH₂ | | | | |
| Hydantoin(C(O)-(D-Arg-Gly))-c(Cys-Glu-His- | 9.49 | 124 | 2.95 | 2260 |
| D-Phe-Arg-Trp-Cys)-NH₂ | | | | |
| Hydantoin(C(O)-(Gly-D-Arg))-c(Cys-Glu-His- | 4.30 | 78.0 | 1.77 | 4540 |
| D-Phe-Arg-Trp-Cys)-NH₂ | | | | |
| Hydantoin(C(O)-(Arg-Gly))-c(Cys-Glu-His-D- | 8.59 | 94.1 | 2.44 | 7760 |
| Phe-Arg-Trp-Cys)-NH₂ | | | | |
| Hydantoin(C(O)-(Gly-Arg))-c(Cys-Glu-His-D- | 5.68 | 55.5 | 2.44 | 4220 |
| Phe-Arg-Trp-Cys)-NH₂ | | | | |
| Hydantoin(C(O)-(Arg-Gly))-c(Cys-D-Ala-His- | 2.65 | 41.3 | 4.17 | 650 |
| D-Phe-Arg-Trp-Cys)-NH₂ | | | | |
| Hydantoin(C(O)-(D-Arg-Gly))-c(Cys-D-Ala- | 3.52 | 48.7 | 5.78 | 872 |
| His-D-Phe-Arg-Trp-Cys)-NH₂ | | | | |
| Hydantoin(C(O)-(Gly-D-Arg))-c(Cys-D-Ala- | 3.51 | 29.2 | 6.04 | 914 |
| His-D-Phe-Arg-Trp-Cys)-NH₂ (SEQ ID | | | | |
| NO288) | | | | |
| Hydantoin(C(O)-(Gly-Arg))-c(Cys-D-Ala-His- | 1.14 | 01.7 | 4.53 | 783 |
| D-Phe-Arg-Trp-Cys)-NH₂ | | | | |
| Hydantoin(C(O)-(Arg-Gly))-c(Cys-D-Ala-His- | 11.9 | 7.43 | 0.195 | 14.6 |
| D-2-Nal-Arg-Trp-Cys)-NH₂ | | | | |

TABLE 7C - Binding Constants for Formula (VII) Examples

| | | | | |
|---|---|---|---|---|
| Formula (VII) Compounds | | | | |
| c[Hydantoin(C(O)-(Aib-Cys))-D-Ala-His-D- | 47.6 | 1100 | 47.1 | >10000 |
| Phe-Arg-Trp-Cys]-NH₂ | | | | |
| c[Hydantoin(C(O)-(Val-Cys))-D-Ala-His-D- | 21.2 | 730 | 34.5 | >10000 |
| Phe-Arg-Trp-Cys]-NH₂ | | | | |
| c[Hydantoin(C(O)-(Leu-Cys))-D-Ala-His-D- | 47.4 | 1550 | 27.9 | >10000 |
| Phe-Arg-Trp-Cys]-NH₂ | | | | |
| c[Hydantoin(C(O)-(Ile-Cys))-D-Ala-His-D-Phe- | 53.4 | 1760 | 41.6 | >10000 |
| Arg-Trp-Cys]-NH₂ | | | | |
| c[Hydantoin(C(O)-(A6c-Cys))-D-Ala-His-D- | 38.5 | 1760 | 53.2 | 9270 |
| Phe-Arg-Trp-Cys]-NH₂ | | | | |
| c[Hydantoin(C(O)-(Gly-Cys))-Glu-His-D-Phe- | 15.6 | 305 | 8.92 | 3070 |
| Arg-Trp-Cys]-NH₂ | | | | |

TABLE 8
Radioligand Binding Assay Data for Selected Compounds

| | Ki | Ki | Ki | Ki |
|---|---|---|---|---|
| Compound | hMC1-R | hMC3-R | hMC4-R | hMC5-R |
| Ac-Tyr-Arg-c(Cys-D-Ala-His- | 8.53 | 21.2 | 3.72 | 714 |
| D-Phe-Arg-Trp-Cys)-NH₂ | | | | |
| Ac-2-Nal-Arg-c(Cys-D-Ala-His- | 6.09 | 34.9 | 2.02 | 864 |
| D-Phe-Arg-Trp-Cys)-NH₂ | | | | |
| Ac-1-Nal-Arg-c(Cys-D-Ala-His- | 6.27 | 36.4 | 1.53 | 888 |
| D-Phe-Arg-Trp-Cys)-NH₂ | | | | |
| Ac-Phe-Arg-c(Cys-D-Ala-His- | 1.48 | 14.8 | 2.34 | 491 |
| D-Phe-Arg-Trp-Cys)-NH₂ | | | | |
| Ac-Trp-Arg-c(Cys-D-Ala-His- | 4.7 | 42 | 2.25 | 1470 |
| D-Phe-Arg-Trp-Cys)-NH₂ | | | | |
| Ac-Pff-Arg-c(Cys-D-Ala-His- | 0.323 | 1.33 | 1.95 | 786 |
| D-Phe-Arg-Trp-Cys)-NH₂ | | | | |

### Melanocortin Functional Activity and Selectivity

The compounds of the present invention will interact preferentially (i.e., selectively) to MC-4 relative to the other melanocortin receptors. Selectivity is particularly important when the compounds are administered to humans or other animals to minimize the number of side effects associated with their administration. MC-4 selectivity of a compound is defined herein as the ratio of the EC₅₀ of the compound for an MC-1 receptor (EC₅₀-MC-1) over the EC₅₀ of the compound for the MC-3 (EC₅₀-MC-3)/MC-4 (EC₅₀-MC-4) receptor, the EC₅₀ values being measured as described above. The formulas are as follows:

MC-3 selectivity=[EC₅₀-MC-1]/[EC₅₀-MC-3]

MC-4 selectivity=[EC₅₀-MC-1]/[EC₅₀-MC-4]

A compound is defined herein as being “selective for the MC-3 receptor” when the above mentioned ratio “MC-3-selectivity” is at least about 10, preferably at least about 100, and more preferably at least about 500.

A compound is defined herein as being “selective for the MC-4 receptor” when the above mentioned ratio “MC-4-selectivity” is at least about 10, preferably at least about 100, and more preferably at least about 500.

One skilled in the art would know that procedures similar to those described herein may be used to assay the binding activities of the compounds of the invention to melanocortin receptor molecules.

cyclic AMP Bioassay

Intracellular cyclic AMP (cAMP) levels were determined by an electrochemiluminescence (ECL) assay (Meso Scale Discovery®, Gaithersburg, Md.; referred to hereinafter as MSD). CHO-K1 cells stably expressing the hMC receptor subtypes were suspended in RMPI 1640® assay buffer (RMPI 1640 buffer contains 0.5 mM isobutylmethylxanthine (IBMX), and 0.2% protein cocktail (MSD blocker A)). Transgenic CHO-K1 cells stably expressing hMC receptor subtypes 1, 3, 4 or 5 were dispensed at a density of approximately 7,000 cells/well in 384-well Multi-Array®plates (MSD) containing integrated carbon electrodes and coated with anti-cAMP antibody. Increasing concentrations of the test compounds were added and the cells were incubated for approximately 40 minutes at approximately 37° C. Following this incubation, lysis buffer (HEPES-buffered saline solution with MgCl₂ and Triton X-100® at ph 7.3) containing 0.2% protein cocktail and 2.5 nM TAG™ ruthenium-labeled cAMP (MSD) was added and the cells were incubated for approximately 90 minutes at room temperature. At the end of the second incubation period read buffer (Tris-buffered solution containing an ECL co-reactant and Triton X-100 at ph 7.8) was added and the cAMP levels in the cell lysates were immediately determined by ECL detection with a Sector Imager 6000 Reader® (MSD). Data were analyzed using a computer-assisted non-linear regression analysis (XL fit; IDBS) and reported as either an EC₅₀ value or a Kb value.

EC₅₀ represents the concentration of an agonist compound needed to obtain 50% of the maximum reaction response, e.g., 50% of the maximum level of cAMP as determined using the assay described above. The Kb value reflects the potency of an antagonist and is determined by Schild analysis. In brief, concentration-response curves of an agonist are carried out in the presence of increasing concentrations of an antagonist. The Kb value is the concentration of antagonist which would produce a 2-fold shift in the concentration-response curve for an agonist. It is calculated by extrapolating the line on a Schild plot to zero on the y-axis.

A selection of compounds was tested using the above-discussed assays and the results are reported in Tables 9, 10, 11, and 12.

TABLE 9
cAMP Bioassay Data for Selected Compounds

| | | | | | |
|---|---|---|---|---|---|
| | | | | | EC₅₀ |
| Compound | EC₅₀ | EC₅₀ | EC₅₀ | EC₅₀ | hMC1-R/ |
| (according to Formula I) | hMC1-R | hMC3-R | hMC4-R | hMC5-R | MC4-R |

Table 9A

| | | | | | |
|---|---|---|---|---|---|
| Ac-Arg-c(Cys-D- | 5.79 | 5.25 | 0.313 | 1630 | 18.0 |
| Ala-His-D-Phe- | | | | | |
| Arg-Trp-Cys)-NH₂ | | | | | |
| Ac-D-Arg-c(Cys-D- | 6.17 | 5.6 | 0.397 | 1020 | 16.0 |
| Ala-His-D-Phe- | | | | | |
| Arg-Trp-Cys)-NH₂ | | | | | |
| Ac-D-Arg-c(Cys-D- | 26.5 | 10.5 | 0.493 | 2440 | 54.0 |
| Ala-His-D-Phe- | | | | | |
| Arg-Trp-Pen)-NH₂ | | | | | |
| Ac-D-Arg-c(Cys- | 8.43 | 32.4 | 0.959 | 2140 | 9.0 |
| His-D-Phe-Arg- | | | | | |
| Trp-Gaba-Pen)-NH₂ | | | | | |
| Ac-Arg-c(Cys-His- | 4.23 | 8.09 | 0.719 | 23.2 | 6.0 |
| D-Phe-Arg-Trp- | | | | | |
| Gaba-Pen)-NH₂ | | | | | |
| Ac-Arg-c(Cys-D- | 48.3 | 13.3 | 0.79 | 10000 | 61.0 |
| Ala-His-D-Phe- | | | | | |
| Arg-Trp-Pen)-NH₂ | | | | | |
| Ac-D-Arg-c(Asp- | 1.48 | 5.76 | 0.078 | 297 | 19.0 |
| His-D-Phe-Arg- | | | | | |
| Trp-Ala-Lys)-NH₂ | | | | | |
| Ac-Arg-c(Asp-His- | 1.39 | 2.89 | 0.055 | 467 | 25.0 |
| D-Phe-Arg-Trp- | | | | | |
| Ala-Lys)-NH₂ | | | | | |

Table 9B

| | | | | | |
|---|---|---|---|---|---|
| Ac-Nle-c(Cys-D-Ala-His- | 2.4 | 0.33 | 0.078 | 420 | 31 |
| D-Phe-Arg-Trp-Cys)-NH₂ | | | | | |
| D-Phe-c(Cys-His-D- | 0.35 | 1.1 | 0.11 | 0.37 | 3 |
| (Et)Tyr-Arg-Trp-β-Ala-D- | | | | | |
| Cys)-Thr-NH₂ | | | | | |
| Nle-c(Cys-His-D-Phe-Arg- | 0.31 | 0.27 | 0.018 | 3.1 | 17 |
| Trp-Apn-Cys)-NH₂ | | | | | |
| Ac-Nle-c(Cys-His-D-Phe- | 0.28 | 0.24 | 0.028 | 3.9 | 10 |
| Arg-Trp-Gaba-Pen)-NH₂ | | | | | |
| Nle-c(Cys-His-D-Phe-Arg- | 0.37 | 0.1 | 0.021 | 1.7 | 18 |
| Trp-Gaba-Cys)-NH₂ | | | | | |
| Ac-Nle-c(Asp-His-D-Phe- | 0.834 | 0.145 | 0.128 | 2.79 | 6.52 |
| Arg-Trp-β-Ala-Lys)-NH₂ | | | | | |
| Ac-Nle-c(Cys-His-D-Phe- | 0.76 | 0.199 | 0.0492 | 1.73 | 15.45 |
| Arg-Trp-Apn-Cys)-NH₂ | | | | | |
| Ac-Cha-c(Asp-His-D-Phe- | 3.26 | 0.189 | 0.0949 | 30.2 | 34.35 |
| Arg-Trp-Gaba-Lys)-NH₂ | | | | | |
| Ac-Nle-c(Asp-His-D-Phe- | 1.37 | 0.628 | 0.131 | 3.48 | 10.46 |
| Arg-Trp-Gaba-Lys)-NH₂ | | | | | |
| Ac-hCha-c(Asp-His-D-Phe- | 2.27 | 3.32 | 7.24 | 415 | 0.31 |
| Arg-Trp-Gaba-Lys)-NH₂ | | | | | |
| Ac-Nle-c(Pen-D-Ala-His- | ND | 1.89 | 0.531 | ND | ND |
| D-Phe-Arg-Trp-Cys)-NH₂ | | | | | |
| Ac-Nle-c(Cys-D-Ala-His- | 14.3 | 2.03 | 0.183 | 2240 | 78.14 |
| D-Phe-Arg-Trp-Pen)-NH₂ | | | | | |
| D-Phe-c(Cys-His-D- | 0.345 | 2.71 | 5376 | 2.38 | 0.06 |
| (Et)Tyr-hArg-Trp-β-Ala-D- | | | | | |
| Cys)-Thr-NH₂ | | | | | |
| D-Phe-c(Cys-His-D- | 0.685 | 81.8 | 86.9 | 31.8 | 0.01 |
| (Et)Tyr-hArg-Bip-β-Ala-D- | | | | | |
| Cys)-Thr-NH₂ | | | | | |
| Ac-Nle-c(Asp-D-Ala-His- | 0.931 | 3.22 | 1.65 | >10000 | 0.56 |
| D-Phe-Arg-Bal-Lys)-NH₂ | | | | | |
| Ac-Nle-c(Cys-D-Leu-His- | 3.24 | 0.465 | 0.0915 | 78.5 | 35.41 |
| D-Phe-Arg-Trp-Cys)-NH₂ | | | | | |
| Ac-Nle-c(Cys-D-Cha-His- | 0.819 | 0.541 | 0.453 | 45.3 | 1.81 |
| D-Phe-Arg-Trp-Cys)-NH₂ | | | | | |

| | | | | |
|---|---|---|---|---|
| Compound | EC50 | Kb | Kb | EC50 |
| (according to Formula I) | hMC1-R | hMC3-R | MC4-R | hMC5-R |

Table 9C

| | | | | |
|---|---|---|---|---|
| Ac-Nle-c(Cys-D-Ala-His-D-2- | 17.6 | 12.4 | 38.8 | 11.8 |
| Nal-Arg-Trp-Cys)-NH₂ | | | | |
| Ac-Nle-c(Asp-His-D-2-Nal-Arg- | 0.619 | 2.98 | 0.109 | 0.189 |
| Trp-Ala-Lys)-NH₂ | | | | |
| Ac-Nle-c(Asp-His-D-2-Nal-Arg- | 0.913 | 0.536 | 0.346 | 0.489 |
| Trp-β-Ala-Lys)-NH₂ | | | | |
| Ac-Nle-c(Cys-His-D-2-Nal-Arg- | 0.231 | 18.4 | 0.782 | 0.153 |
| Trp-Gaba-Cys)-NH₂ | | | | |
| Ac-Nle-c(Cys-His-D-2-Nal-Arg- | 0.581 | 10.8 | 0.967 | 0.126 |
| Trp-Ahx-Cys)-NH₂ | | | | |
| Ac-hPhe-c(Asp-His-D-2-Nal-Arg- | 0.413 | 9.32 | 0.824 | 0.307 |
| Trp-Gaba-Lys)-NH₂ | | | | |
| Ac-Cha-c(Asp-His-D-2-Nal-Arg- | 1.27 | 3.02 | 0.442 | 0.736 |
| Trp-Gaba-Lys)-NH₂ | | | | |
| Ac-Nle-c(Cys-D-Ala-His-D-2- | 383 | 61.5 | 53.6 | 2842 |
| Nal-Arg-1-Nal-Cys)-NH₂ | | | | |

Table 9D

| | | | | |
|---|---|---|---|---|
| Ac-Arg-c(Cys-D-Ala-His-D-2- | 193 | 5.72 | 1.58 | 1111 |
| Nal-Arg-Trp-Cys)-NH₂ | | | | |

ND = not determined

Table 10A

| | EC₅₀ | Kb | Kb | EC₅₀ |
|---|---|---|---|---|
| Compound (according to Formula III) | hMC1-R | hMC3-R | hMC4-R | hMC5-R |
| Ac-c(Cys-Glu-His-D-2-Nal-Arg-Trp-Ala- | 66.1 | 33.4 | 0.687 | 6.84 |
| Cys)-Pro-Pro-Lys-Asp-NH₂ | | | | |
| Tyr-Gly-Arg-Lys-Lys-Arg-Arg-Gln-Arg- | ND | 4500 | 105 | ND |
| Arg-Arg-Nle-c(Asp-His-D-2-Nal-Arg-Trp- | | | | |
| Lys)-NH₂ | | | | |
| Tyr-Gly-Arg-Lys-Lys-Arg-Arg-Gln-Arg- | ND | 395 | 16.8 | ND |
| Arg-Arg-Doc-Nle-c(Asp-His-D-2-Nal-Arg- | | | | |
| Trp-Lys)-NH₂ | | | | |
| Nle-c(Asp-His-D-2-Nal-Arg-Trp-Lys)-β- | ND | 207 | 18.5 | ND |
| Ala-Tyr-Gly-Arg-Lys-Lys-Arg-Arg-Gln- | | | | |
| Arg-Arg-Arg-NH₂ | | | | |
| Ac-Nle-c(Asp-His-D-2-Nal-Arg-Trp-Lys)-β- | ND | 220 | 4.07 | ND |
| Ala-Tyr-Gly-Arg-Lys-Lys-Arg-Arg-Gln- | | | | |
| Arg-Arg-Arg-NH₂ | | | | |
| H-Nle-c(Asp-His-D-2-Nal-Arg-Trp-Lys)- | ND | 261 | 3.11 | ND |
| Doc-Doc-Tyr-Gly-Arg-Lys-Lys-Arg-Arg- | | | | |
| Gln-Arg-Arg-Arg-NH₂ | | | | |
| Ac-Nle-c(Asp-His-D-2-Nal-Arg-Trp-Lys)- | ND | 14.1 | 22.8 | ND |
| Pro-Pro-Lys-Asp-Tyr-Gly-Arg-Lys-Lys- | | | | |
| Arg-Arg-Gln-Arg-Arg-Arg-NH₂ | | | | |
| Ac-c(Cys-Glu-His-D-2-Nal-Arg-Trp-Gly- | ND | 233 | 26.0 | ND |
| Cys)-Pro-Pro-Lys-Asp-Tyr-Gly-Arg-Lys- | | | | |
| Lys-Arg-Arg-Gln-Arg-Arg-Arg-NH₂ | | | | |
| Ac-Nle-c(Asp-His-D-2-Nal-Arg-Trp-Lys)-β- | 1.39 | 16.2 | 7.94 | 0.839 |
| Ala-β-Ala-Tyr-Gly-Arg-Lys-Lys-Arg-Arg- | | | | |
| Gln-Arg-Arg-Arg-NH₂ | | | | |
| Ac-Nle-c(Asp-His-D-2-Nal-Arg-Trp-Lys)- | 3.65 | 19.4 | 3.73 | 1.61 |
| Pro-Pro-Lys-Asp-Doc-Tyr-Gly-Arg-Lys- | | | | |
| Lys-Arg-Arg-Gln-Arg-Arg-Arg-NH₂ | | | | |
| Ac-c(Cys-Glu-His-D-2-Nal-Arg-Trp-Gly- | ND | 17.7 | 1.49 | ND |
| Cys)-Pro-Pro-Lys-Asp-Doc-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Trp-Ala- | 6.3 | 70.0 | 1.66 | 38.2 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Trp-Ala- | 12.1 | 30.3 | 1.81 | 70.0 |
| Cys)-Pro-Pro-Lys-Asp-Doc-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-2-Nal-Ala- | 33.6 | 140 | 12.2 | 66.9 |
| Cys)-Pro-Pro-Lys-Asp-NH₂ | | | | |
| Ac-c(Cys-Glu-His-D-2-Nal-Arg-1-Nal-Ala- | 269 | 105 | 5.92 | 104 |
| Cys)-Pro-Pro-Lys-Asp-NH₂ | | | | |
| Ac-c Cys-Glu-His-D-2-Nal-Arg-Bal-Ala- | 690 | 70.7 | 4.56 | 177 |
| Cys)-Pro-Pro-Lys-Asp-NH₂ | | | | |
| Ac-Nle-c(Asp-His-D-2-Nal-Arg-Trp-Lys)- | 3.23 | 8.97 | 4.61 | 2.86 |
| Doc-Doc-Tyr-Gly-Arg-Lys-Lys-Arg-Arg- | | | | |
| Gln-Arg-Arg-Arg-NH₂ | | | | |
| Ac-c(Cys-Glu-His-D-2-Nal-Arg-Bal-Ala- | 52.0 | 170 | 6.14 | 328 |
| Cys)-Pro-Pro-Lys-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-Bal-Ala- | 146 | 104 | 32.0 | 1400 |
| Cys)-Pro-Pro-Lys-Asp-β-Ala-Gly-Arg-Arg- | | | | |
| Arg-Arg-Arg-Gln-Arg-Arg-Arg-NH₂ | | | | |
| Ac-c(Cys-Glu-His-D-2-Nal-Arg-Bal-Ala- | 114 | 44.6 | 28.4 | 879 |
| Cys)-Pro-Pro-Lys-Asp-β-Ala-Tyr-Gly-Arg- | | | | |
| Arg-Arg-Arg-Arg-Gln-Arg-Arg-Arg-NH₂ | | | | |
| Ac-c(Cys-Glu-His-D-2-Nal-Arg-Bal-Ala- | 67.1 | 439 | 46.5 | 582 |
| Cys)-Pro-Pro-Lys-Asp-β-Ala-Tyr-Aib-Arg- | | | | |
| Lys-Lys-Arg-Arg-Gln-Arg-Arg-Arg-NH₂ | | | | |
| Ac-c(Cys-Glu-His-D-2-Nal-Arg-1-Nal-Ala- | 144 | 116 | 8.93 | 819 |
| Cys)-Pro-Pro-Arg-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-1-Nal-Ala- | 36.0 | 46.5 | 11.4 | 56.1 |
| Cys)-Pro-Pro-Lys-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-1-Nal-Ala- | 93.0 | 71 | 15.9 | >10000 |
| Cys)-Pro-Pro-Lys-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-2-Nal-Ala- | 39.7 | 30.9 | 6.66 | 501 |
| Cys)-Pro-Pro-Arg-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-2-Nal-Ala- | 35.2 | 22.9 | 12.6 | 199 |
| Cys)-Pro-Pro-Lys-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-2-Nal-Ala- | 29.1 | 13.6 | 13.4 | 204 |
| Cys)-Pro-Pro-Lys-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-Bal-Ala- | 86.1 | 41.7 | 19.4 | 2360 |
| Cys)-Pro-Pro-Arg-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-Bal-Ala- | 38.3 | 20.2 | 21.2 | >10000 |
| Cys)-Pro-Pro-Arg-Asp-β-Ala-Arg-Arg-Arg- | | | | |
| Arg-Arg-Gln-Arg-Arg-Arg-NH₂ | | | | |
| Ac-c(Cys-Glu-His-D-2-Nal-Arg-Bal-Ala- | 68.6 | 153 | 33.2 | >10000 |
| Cys)-Pro-Pro-Lys-Asp-β-Ala-Arg-Arg-Arg- | | | | |
| Arg-Arg-Arg-Gln-Arg-Arg-Arg-NH₂ | | | | |
| Ac-c(Cys-Glu-His-D-2-Nal-Arg-1-Nal-Ala- | 70.4 | 286 | 18.6 | >10000 |
| Cys)-Pro-Pro-Lys-Asp-β-Ala-Arg-Arg-Lys- | | | | |
| Arg-Arg-Gln-Arg-Arg-Arg-NH₂ | | | | |
| Ac-c(Cys-Glu-His-D-2-Nal-Arg-1-Nal-Ala- | 33.1 | 65.1 | 15.3 | 1720 |
| Cys)-Pro-Pro-Lys-Asp-β-Ala-Arg-Lys-Arg- | | | | |
| Arg-Arg-Gln-Arg-Arg-Arg-NH₂ | | | | |
| Ac-c(Cys-Glu-His-D-2-Nal-Arg-2-Nal-Ala- | 88.2 | 10.6 | 17.4 | 514 |
| Cys)-Pro-Pro-Lys-Asp-β-Ala-Arg-Arg-Lys- | | | | |
| Arg-Arg-Gln-Arg-Arg-Arg-NH₂ | | | | |
| Ac-c(Cys-Glu-His-D-2-Nal-Arg-2-Nal-Ala- | 58.7 | 39.3 | 10.3 | 460 |
| Cys)-Pro-Pro-Lys-Asp-β-Ala-Tyr-Gly-Arg- | | | | |
| Arg-Lys-Arg-Arg-Gln-Arg-Arg-Arg-NH₂ | | | | |
| Ac-c(Cys-Glu-His-D-2-Nal-Arg-2-Nal-Ala- | 45.4 | 12.7 | 12.7 | 162 |
| Cys)-Pro-Pro-Lys-Asp-β-Ala-Gly-Arg-Arg- | | | | |
| Lys-Arg-Arg-Gln-Arg-Arg-Arg-NH₂ | | | | |
| Ac-c(Cys-Glu-His-D-2-Nal-Arg-2-Nal-Ala- | 309 | 22.8 | 17.1 | 570 |
| Cys)-Pro-Pro-Lys-Asp-β-Ala-Gly-Arg-Lys- | | | | |
| Arg-Arg-Arg-Gln-Arg-Arg-Arg-NH₂ | | | | |
| Ac-Nle-c(Cys-D-Ala-His-D-Phe-Arg-Trp- | 7.86 | 10.5 | 0.843 | 4900 |
| Cys)-β-Ala-Tyr-Gly-Arg-Arg-Arg-Arg-Arg- | | | | |
| Gln-Arg-Arg-Arg-NH₂ | | | | |
| Ac-c(Cys-Glu-His-D-2-Nal-Arg-2-Nal-Ala- | 29.7 | 25.6 | 7.37 | 82.9 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Trp-Ala- | 15.2 | 14.6 | 4.52 | 36.8 |
| Cys)-Pro-Pro-Lys-Asp-βAla-Tyr-Gly-Arg- | | | | |
| Lys-Lys-Arg-Gln-Arg-Arg-Arg-Arg-NH₂ | | | | |
| Ac-c(Cys-Glu-His-D-2-Nal-Arg-Trp-Ala- | 6.7 | 9.38 | 11.7 | 46.2 |
| Cys)-Pro-Pro-Lys-Asp-β-Ala-Tyr-Gly-Arg- | | | | |
| Lys-Lys-Gln-Arg-Arg-Arg-Arg-Arg-NH₂ | | | | |
| Ac-c(Cys-Glu-His-D-2-Nal-Arg-Trp-Ala- | 7.9 | 41.7 | 10.9 | 62.4 |
| Cys)-Pro-Pro-Lys-Asp-β-Ala-Tyr-Gly-Arg- | | | | |
| Lys-Gln-Lys-Arg-Arg-Arg-Arg-Arg-NH₂ | | | | |
| Ac-c(Cys-Glu-His-D-2-Nal-Arg-Trp-Ala- | 16.9 | 36.0 | 7.12 | 58.9 |
| Cys)-Pro-Pro-Lys-Asp-β-Ala-Tyr-Gly-Arg- | | | | |
| Lys-Lys-Arg-Arg-Arg-Arg-Gln-Arg-NH₂ | | | | |
| Ac-c(Cys-Glu-His-D-2-Nal-Arg-Trp-Ala- | 16.4 | 20.8 | 7.31 | 44.2 |
| Cys)-Pro-Pro-Lys-Asp-β-Ala-Tyr-Gly-Arg- | | | | |
| Lys-Lys-Arg-Arg-Arg-Gln-Arg-Arg-NH₂ | | | | |
| Ac-c(Cys-Glu-His-D-2-Nal-Arg-Trp-Ala- | 12.0 | 13.7 | 9.38 | 54.2 |
| Cys)-Pro-Pro-Lys-Asp-β-Ala-Tyr-Gly-Arg- | | | | |
| Gln-Lys-Lys-Arg-Arg-Arg-Arg-Arg-NH₂ | | | | |
| Ac-c(Cys-Glu-His-D-2-Nal-Arg-Trp-Ala- | 7.5 | 12.2 | 7.61 | 51.7 |
| Cys)-Pro-Pro-Lys-Asp-β-Ala-Tyr-Gly-Arg- | | | | |
| Lys-Lys-Arg-Arg-Arg-Arg-Arg-Gln-NH₂ | | | | |
| Ac-c(Cys-Glu-His-D-2-Nal-Arg-1-Nal-Ala- | 43.3 | 215 | 5.87 | 1286 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |
| Ac-c(Cys-Glu-His-D-2-Nal-Arg-Bal-Ala- | 37.9 | 112 | 41.1 | 1798 |
| Cys)-Pro-Pro-Lys-Asp-β-Ala-Tyr-Gly-Arg- | | | | |
| Lys-Lys-Arg-Arg-Gln-Arg-Arg-Arg-NH₂ | | | | |

Table 10B

| | EC50 | EC50 | EC50 | EC50 |
|---|---|---|---|---|
| Compound (according to Formula III) | hMC1-R | hMC3-R | hMC4-R | hMC5-R |
| Ac-Nle-c(Cys-D-Ala-His-D-Phe-Arg-Trp- | 4.70 | 4.56 | 0.634 | 147 |
| Cys)-(Doc)2-Tyr-Gly-Arg-Lys-Lys-Arg- | | | | |
| Arg-Gln-Arg-Arg-Arg-NH₂ | | | | |
| Ac-Nle-c(Cys-D-Ala-His-D-Phe-Arg-Trp- | 5.90 | 7.73 | 1.02 | 2890 |
| Cys)-β-Ala-Tyr-Gly-Arg-Lys-Lys-Arg-Gln- | | | | |
| Arg-Arg-Arg-Arg-NH₂ | | | | |
| Ac-Nle-c(Cys-D-Ala-His-D-Phe-Arg-Trp- | 0.481 | 7.32 | 0.964 | 2010 |
| Cys)-β-Ala-Gly-Arg-Arg-Arg-Arg-Arg-Gln- | | | | |
| Arg-Arg-Arg-NH₂ | | | | |
| Ac-Nle-c(Cys-D-Ala-His-D-Phe-Arg-Trp- | 7.15 | 9.37 | 1.25 | 1570 |
| Cys)-β-Ala-Arg-Arg-Arg-Arg-Arg-Gln-Arg- | | | | |
| Arg-Arg-NH₂ | | | | |

Table 10C

| | EC₅₀ | Kb | Kb | EC₅₀ |
|---|---|---|---|---|
| Compound (according to Formula IV) | hMC1-R | hMC3-R | hMC4-R | hMC5-R |
| Ac-c(Cys-Glu-His-D-2-Nal-Arg-2-Nal-β- | ND | ND | ND | ND |
| Ala-Cys)-Pro-Pro-Lys-Asp-NH₂ | | | | |
| Ac-c(Cys-Glu-His-D-2-Nal-Arg-2-Nal-Aib- | 770 | 221 | 4.52 | 869 |
| Cys)-Pro-Pro-Lys-Asp-NH₂ | | | | |
| Ac-c(Cys-Glu-His-D-2-Nal-Arg-1-Nal-Ala- | 29 | 22.6 | 16.7 | 173 |
| Cys)-Pro-Pro-Lys-Asp-β-Ala-Tyr-Gly-Arg- | | | | |
| Arg-Lys-Arg-Arg-Gln-Arg-Arg-Arg-NH₂ | | | | |
| Ac-c(Cys-Glu-His-D-2-Nal-Arg-1-Nal-Ala- | 102 | 26.3 | 14.6 | 261 |
| Cys)-Pro-Pro-Lys-Asp-β-Ala-Tyr-Gly-Arg- | | | | |
| Lys-Arg-Arg-Arg-Gln-Arg-Arg-Arg-NH₂ | | | | |
| Ac-c(Cys-Glu-His-D-2-Nal-Arg-1-Nal-Ala- | 26.6 | 101 | 9.34 | 351 |
| Cys)-Pro-Pro-Lys-Asp-β-Ala-Gly-Arg-Arg- | | | | |
| Lys-Arg-Arg-Gln-Arg-Arg-Arg-NH₂ | | | | |
| Ac-c(Cys-Glu-His-D-2-Nal-Arg-1-Nal-Ala- | 45.5 | 181 | 6.35 | 149 |
| Cys)-Pro-Pro-Lys-Asp-β-Ala-Gly-Arg-Lys- | | | | |
| Arg-Arg-Arg-Gln-Arg-Arg-Arg-NH₂ | | | | |
| Ac-c(Cys-Glu-His-D-2-Nal-Arg-2-Nal-Ala- | 23.7 | 9.22 | 5.87 | 39.7 |
| Cys)-Pro-Pro-Lys-Asp-β-Ala-Arg-Lys-Arg- | | | | |
| Arg-Arg-Gln-Arg-Arg-Arg-NH₂ | | | | |
| Ac-c(Cys-Glu-His-D-2-Nal-Arg-2-Nal-Ala- | 34.7 | 15.0 | 8.68 | 28.2 |
| Cys)-Pro-Pro-Lys-Asp-β-Ala-Tyr-Gly-Arg- | | | | |
| Lys-Arg-Arg-Arg-Gln-Arg-Arg-Arg-NH₂ | | | | |
| Ac-c(Cys-Glu-His-D-2-Nal-Arg-Bal-Ala- | 19.1 | 106 | 4.59 | 100 |
| Cys)-Pro-Pro-Lys-Asp-β-Ala-Arg-Lys-Arg- | | | | |
| Arg-Arg-Gln-Arg-Arg-Arg-NH₂ | | | | |
| Ac-c(Cys-Glu-His-D-2-Nal-Arg-Bal-Ala- | 19.8 | 37.8 | 8.43 | 158 |
| Cys)-Pro-Pro-Lys-Asp-β-Ala-Tyr-Gly-Arg- | | | | |
| Arg-Lys-Arg-Arg-Gln-Arg-Arg-Arg-NH₂ | | | | |
| Ac-c(Cys-Glu-His-D-2-Nal-Arg-Bal-Ala- | 11.2 | 52.1 | 9.45 | 95.7 |
| Cys)-Pro-Pro-Lys-Asp-β-Ala-Tyr-Gly-Arg- | | | | |
| Lys-Arg-Arg-Arg-Gln-Arg-Arg-Arg-NH₂ | | | | |
| Ac-c(Cys-Glu-His-D-2-Nal-Arg-Bal-Ala- | 33.8 | 93.6 | 4.42 | 89.5 |
| Cys)-Pro-Pro-Lys-Asp-β-Ala-Gly-Arg-Arg- | | | | |
| Lys-Arg-Arg-Gln-Arg-Arg-Arg-NH₂ | | | | |
| Ac-c(Cys-Glu-His-D-2-Nal-Arg-Bal-Ala- | 232 | 68.8 | 10.0 | 250 |
| Cys)-Pro-Pro-Lys-Asp-β-Ala-Arg-Arg-Lys- | | | | |
| Arg-Arg-Gln-Arg-Arg-Arg-NH₂ | | | | |
| Ac-c(Cys-Glu-His-D-2-Nal-Arg-Bal-Ala- | 32.2 | 98.3 | 5.23 | 194 |
| Cys)-Pro-Pro-Lys-Asp-β-Ala-Gly-Arg-Lys- | | | | |
| Arg-Arg-Arg-Gln-Arg-Arg-Arg-NH₂ | | | | |

Table 10D

| | EC50 | EC50 | EC50 | EC50 |
|---|---|---|---|---|
| Compound (according to Formula IV) | hMC1-R | hMC3-R | hMC4-R | hMC5-R |
| Ac-Nle-c(Cys-D-Ala-His-D-Phe-Arg-Trp- | 5.66 | 4.70 | 0.422 | 1551 |
| Cys)-β-Ala-Tyr-Gly-Arg-Arg-Lys-Arg-Arg- | | | | |
| Gln-Arg-Arg-Arg-NH₂ | | | | |
| Ac-Nle-c(Cys-D-Ala-His-D-Phe-Arg-Trp- | 7.57 | 4.18 | 0.600 | 1792 |
| Cys)-β-Ala-Tyr-Gly-Arg-Lys-Arg-Arg-Arg- | | | | |
| Gln-Arg-Arg-Arg-NH₂ | | | | |
| Ac-Nle-c(Cys-D-Ala-His-D-Phe-Arg-Trp- | 2.36 | 2.74 | 0.260 | 500 |
| Cys)-β-Ala-Gly-Arg-Arg-Lys-Arg-Arg- | | | | |
| Gln-Arg-Arg-Arg-NH₂ | | | | |
| Ac-Nle-c(Cys-D-Ala-His-D-Phe-Arg-Trp- | 2.81 | 3.29 | 0.298 | 566 |
| Cys)-β-Ala-Gly-Arg-Lys-Arg-Arg-Arg- | | | | |
| Gln-Arg-Arg-Arg-NH₂ | | | | |
| Ac-Nle-c(Cys-D-Ala-His-D-Phe-Arg-Trp- | 1.86 | 1.39 | 0.367 | 165 |
| Cys)-β-Ala-Arg-Arg-Lys-Arg-Arg-Gln- | | | | |
| Arg-Arg-Arg-NH₂ | | | | |
| Ac-Nle-c(Cys-D-Ala-His-D-Phe-Arg-Trp- | 2.06 | 1.61 | 0.394 | 199 |
| Cys)-β-Ala-Arg-Lys-Arg-Arg-Arg-Gln- | | | | |
| Arg-Arg-Arg-NH₂ | | | | |

ND = not determined

TABLE 11
Intracellular Cyclic AMP (cAMP) Levels for Formula (I) Examples

| | | | |
|---|---|---|---|
| EC₅₀ | EC₅₀ | EC₅₀ | EC₅₀ |
| hMC1 | hMC3 | hMC4 | hMC5 |

Table 11A

| | | | | |
|---|---|---|---|---|
| Formula (V) Compounds | | | | |
| c[Hydantoin(C(O)-(Cys-D-Ala))- | — | 218 | 5.42 | — |
| His-D-Phe-Arg-Trp-Cys]-NH₂ | | | | |
| c[Hydantoin(C(O)-(Glu-D-Ala))- | — | 22.3 | 3.62 | — |
| His-D-Phe-Arg-Trp-Lys]-NH₂ | | | | |
| c[Hydantoin(C(O)-(Glu-D-Ala))- | — | 39.2 | 4.94 | — |
| His-D-Phe-Arg-Trp-Orn]-NH₂ | | | | |
| c[Hydantoin(C(O)-(Glu-D-Ala))- | 56.7 | 18.2 | 0.182 | >10000 |
| His-D-Phe-Arg-Trp-Dap]-NH₂ | | | | |
| c[Hydantoin(C(O)-(Asp-D-Ala))- | 56.6 | 88.6 | 4.50 | 9300 |
| His-D-Phe-Arg-Trp-Orn]-NH₂ | | | | |
| c[Hydantoin(C(O)-(Asp-D-Ala))- | — | 49.3 | 2.12 | — |
| His-D-Phe-Arg-Trp-Dap]-NH₂ | | | | |

TABLE 11B

| | | | | |
|---|---|---|---|---|
| Formula (VI) Compounds | | | | |
| Hydantoin(C(O)-(Nle-Gly))-c(Cys- | 54.3 | 12.2 | 0.177 | >10000 |
| Glu-His-D-Phe-Arg-Trp-Cys)-NH₂ | | | | |
| Hydantoin(C(O)-(Gly-Gly))-c(Cys- | 124 | 8.05 | 0.214 | >10000 |
| Glu-His-D-Phe-Arg-Trp-Cys)-NH₂ | | | | |
| Hydantoin(C(O)- | — | 4.89 | 1.80 | — |
| (A6c-Nle))-c(Cys-D- | | | | |
| Ala-His-D-Phe-Arg-Trp-Cys)-NH₂ | | | | |
| Hydantoin(C(O)- | — | 2.56 | 1.47 | — |
| (D-Ala-Nle))-c(Cys-D- | | | | |
| Ala-His-D-Phe-Arg-Trp-Cys)-NH₂ | | | | |
| Hydantoin(C(O)- | — | 4.61 | 0.977 | — |
| (Val-Nle))-c(Cys-D- | | | | |
| Ala-His-D-Phe-Arg-Trp-Cys)-NH₂ | | | | |
| Hydantoin(C(O)- | — | 9.68 | 1.83 | — |
| (Leu-Nle))-c(Cys-D- | | | | |
| Ala-His-D-Phe-Arg-Trp-Cys)-NH₂ | | | | |
| Hydantoin(C(O)- | — | 9.97 | 13.9 | — |
| (Cha-Nle))-c(Cys-D- | | | | |
| Ala-His-D-Phe-Arg-Trp-Cys)-NH₂ | | | | |
| Hydantoin(C(O)- | 14.2 | 2.46 | 0.336 | 201 |
| (Gly-Gly))-c(Cys-D- | | | | |
| Ala-His-D-Phe-Arg-Trp-Cys)-NH₂ | | | | |
| Hydantoin(C(O)- | 17.0 | 21.5 | 0.584 | 352 |
| (Nle-Gly))-c(Cys-D- | | | | |
| Ala-His-D-Phe-Arg-Trp-Pen)-NH₂ | | | | |
| Hydantoin(C(O)- | 40.2 | 8.90 | 0.495 | 8300 |
| (Gly-Gly))-c(Cys-D- | | | | |
| Ala-His-D-Phe-Arg-Trp-Pen)-NH₂ | | | | |
| Hydantoin(C(O)- | 17.6 | 2.23 | 0.241 | 516 |
| (Ala-Gly))-c(Cys-D- | | | | |
| Ala-His-D-Phe-Arg-Trp-Cys)-NH₂ | | | | |
| Hydantoin(C(O)- | 4.70 | 2.22 | 0.309 | 355 |
| (D-Ala-Gly))-c(Cys-D- | | | | |
| Ala-His-D-Phe-Arg-Trp-Cys)-NH₂ | | | | |
| Hydantoin(C(O)- | 18.0 | 17.1 | 0.160 | 2710 |
| (D-Arg-Gly))-c(Cys- | | | | |
| Glu-His-D-Phe-Arg-Trp-Cys)-NH₂ | | | | |
| Hydantoin(C(O)- | 12.9 | 10.3 | 0.125 | 7440 |
| (Gly-D-Arg))-c(Cys- | | | | |
| Glu-His-D-Phe-Arg-Trp-Cys)-NH₂ | | | | |
| Hydantoin(C(O)-(Arg-Gly))-c(Cys- | 8.83 | 7.86 | 0.0979 | 4010 |
| Glu-His-D-Phe-Arg-Trp-Cys)-NH₂ | | | | |
| Hydantoin(C(O)-(Gly-Arg))-c(Cys- | 9.97 | 3.63 | 0.0687 | 335 |
| Glu-His-D-Phe-Arg-Trp-Cys)-NH₂ | | | | |
| Hydantoin(C(O)- | 8.81 | 18.2 | 0.503 | 3560 |
| (Arg-Gly))-c(Cys-D- | | | | |
| Ala-His-D-Phe-Arg-Trp-Cys)-NH₂ | | | | |
| Hydantoin(C(O)- | 11.5 | 23.2 | 0.513 | 3950 |
| (D-Arg-Gly))-c(Cys-D-Ala- | | | | |
| His-D-Phe-Arg-Trp-Cys)-NH₂ | | | | |
| Hydantoin(C(O)- | 7.53 | 11.6 | 0.435 | 9840 |
| (Gly-D-Arg))-c(Cys-D-Ala-His- | | | | |
| D-Phe-Arg-Trp-Cys)-NH₂ | | | | |
| Hydantoin(C(O)- | 8.85 | 5.17 | 0.599 | 3610 |
| (Gly-Arg))-c(Cys-D- | | | | |
| Ala-His-D-Phe-Arg-Trp-Cys)-NH₂ | | | | |
| Hydantoin(C(O)- | 96.6 | 13.1 | 21.2 | 103 |
| (Arg-Gly))-c(Cys-D-Ala-His- | | | | |
| D-2-Nal-Arg-Trp-Cys)-NH₂ | | | | |

TABLE 11C

| | | | | |
|---|---|---|---|---|
| Formula (VII) Compounds | | | | |
| c[Hydantoin(C(O)-(Aib-Cys))-D- | — | 6.28 | 0.407 | — |
| Ala-His-D-Phe-Arg-Trp-Cys]-NH₂ | | | | |
| c[Hydantoin(C(O)-(Val-Cys))-D- | — | 3.77 | 0.214 | — |
| Ala-His-D-Phe-Arg-Trp-Cys]-NH₂ | | | | |
| c[Hydantoin(C(O)-(Leu-Cys))-D- | — | 4.72 | 0.428 | — |
| Ala-His-D-Phe-Arg-Trp-Cys]-NH₂ | | | | |
| c[Hydantoin(C(O)-(Ile-Cys))-D- | — | 8.51 | 1.85 | — |
| Ala-His-D-Phe-Arg-Trp-Cys]-NH₂ | | | | |
| c[Hydantoin(C(O)-(A6c-Cys))-D- | — | 5.66 | 1.72 | — |
| Ala-His-D-Phe-Arg-Trp-Cys]-NH₂ | | | | |
| c[Hydantoin(C(O)-(Gly-Cys))-Glu- | 14.5 | 21.8 | 0.576 | 1780 |
| His-D-Phe-Arg-Trp-Cys]-NH₂ | | | | |

TABLE 12
cAMP Bioassay Data for Selected Compounds

| | EC₅₀ | EC₅₀ | EC₅₀ | EC₅₀ |
|---|---|---|---|---|
| | hMC1- | hMC3- | hMC4- | hMC5- |
| Compound | R | R | R | R |
| Ac-Tyr-Arg-c(Cys-D-Ala-His-D- | 6.42 | 2.39 | 0.194 | 1540 |
| Phe-Arg-Trp-Cys)-NH₂ | | | | |
| Ac-2-Nal-Arg-c(Cys-D-Ala-His-D- | 9.66 | 6.11 | 0.275 | 1730 |
| Phe-Arg-Trp-Cys)-NH₂ | | | | |
| Ac-1-Nal-Arg-c(Cys-D-Ala-His-D- | 8.67 | 4.21 | 0.363 | 1320 |
| Phe-Arg-Trp-Cys)-NH₂ | | | | |
| Ac-Trp-Arg-c(Cys-D-Ala-His-D- | 5.78 | 3.95 | 0.219 | 2580 |
| Phe-Arg-Trp-Cys)-NH₂ | | | | |

### In Vivo Studies

Compounds of the present invention can be and were tested for an effect upon dyslipidemia, insulin resistance and/or body weight according to the following procedures. One skilled in the art would know that procedures similar to those described herein may be used to assay the effect of the compounds of the invention upon insulin resistance and/or body weight.

Ligand compounds activating melanocortin receptors tested in the in vivo studies were as follows (Table 13):

TABLE 13

| Ligand Code | Structure |
|---|---|
| Compound A | Ac-Arg-c(Cys-D-Ala-His-D-Phe-Arg-Trp-Cys)-NH₂ |

Compound A was used at a dose of either 50 or 500 nmole/kg/day dissolved at the appropriate concentration in 0.9% NaCl containing 5% DMA, 2% tween-80 and 2% heat inactivated normal rat serum. Sub-cutaneous (sc) infusion pumps (Alzet 2002) were implanted into the rats and used to dose Compound A or the carrier vehicle. The dosage forms were prepared prior to the start of the experiment and used to fill the pumps under sterile conditions. Pumps were primed overnight at 37° C. in sterile saline and implanted on the starting day of the experiment.

Obese Zucker rats were used to study the effects of Compound A upon dyslipidemia, food consumption and body weight. The Zucker rats are obese due to a spontaneous mutation in the leptin gene. In addition to the visible phenotype of obesity, these lep⁻/lep⁻ animals also exhibit hyperinsulinemia, hypertriglyceridemia, hypercholesterolemia and elevated blood pressure.

Three groups of eight obese male Zucker rats (350-400 g) were housed in individual cages and maintained under 12:12 hour light:dark conditions with both food (Harlan Teklad Sterilized Rodent Diet LM-485) and water available ad libitum. Prior to day 1 of the experiment, pumps were primed overnight at 37° C. in sterile saline and Compound A was dissolved at the appropriate concentration in 0.9% NaCl containing 5% DMA, 2% tween-80 and 2% heat inactivated normal rat serum.

On the morning of day 1, the rats were anesthetized using chlorohydrate anesthetic and implanted with a sub-cutaneous infusion pump (Alzet® 2002) just under the skin. Pumps were filled with either Compound A at either 50 or 500 nmole/kg/day, or vehicle (0.9% NaCl containing 5% DMA, 2% tween-80 and 2% heat inactivated normal rat serum). Individual body weight and food and water consumption were measured daily at 0800 hours for 7 days.

On day 7 a right atrial cannula was implanted in the jugular vein under cholorohydrate anesthesia. The animals were fasted overnight and an intravenous (iv) glucose tolerance test was performed. Glucose (1 g/kg) was injected at time 0 and blood samples were taken via the cannula at 0, 2.5, 5, 10, 20 and 40 minutes post-injection. Plasma was collected and assayed for glucose (Glucose (Trinder) Assay; Diagnostic Chemicals Limited, Charlottetown, P.E.I., Canada; Cat #220-32) and insulin (Mercodia Rat Insulin ELISA; Mercodia, Uppsala, Sweden; ALPCO 10-1124-10) content. At the conclusion of the glucose tolerance test and additional serum samples were collected and assayed for triglycerides (Triglyceride L-Type TG H kit: Wako Diagnostics, Richmond, Va.), cholesterol (Cholesterol E assay kit: Wako Diagnostics, Richmond, Va.) and free fatty acids (Fatty Acid HR series NEFA-HR2 kit: Wako Diagnostics, Richmond, Va.).

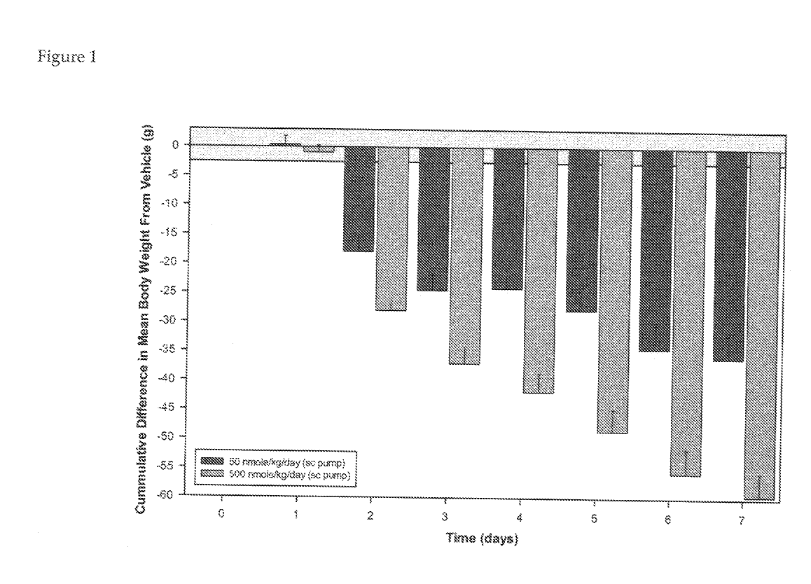

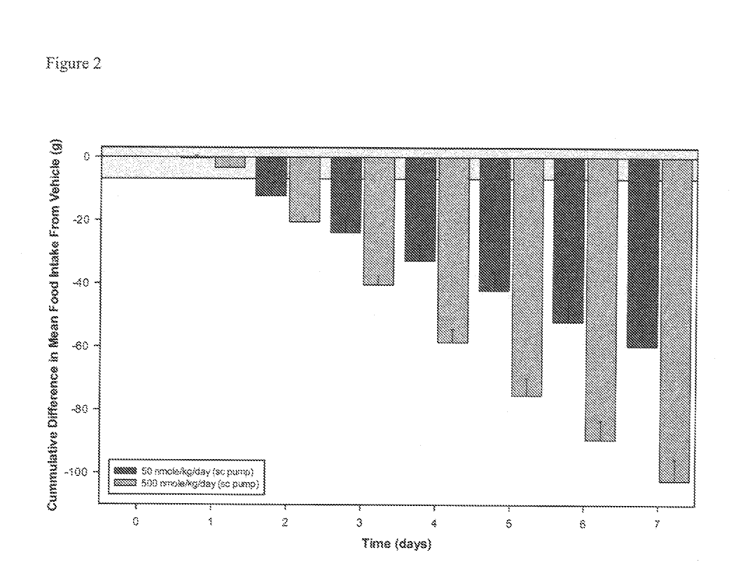

By day 7, treatment with Compound A at a low dose of 50 and a high dose of 500 nmole/kg/day induced a dose-related decrease in body weight gain of 35 g and 60 g, respectively (FIG. 1). A similar dose-related decrease in food intake was also observed by the end of the 7 day treatment period; the low dose group reduced its food intake by 60 g and the high dose group by 100 g (FIG. 2).

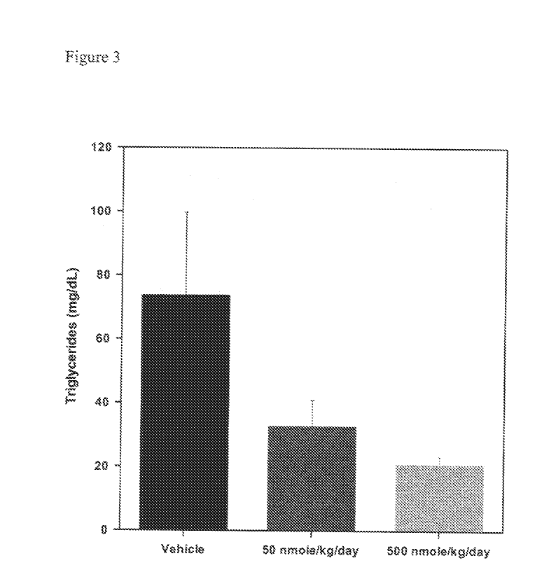

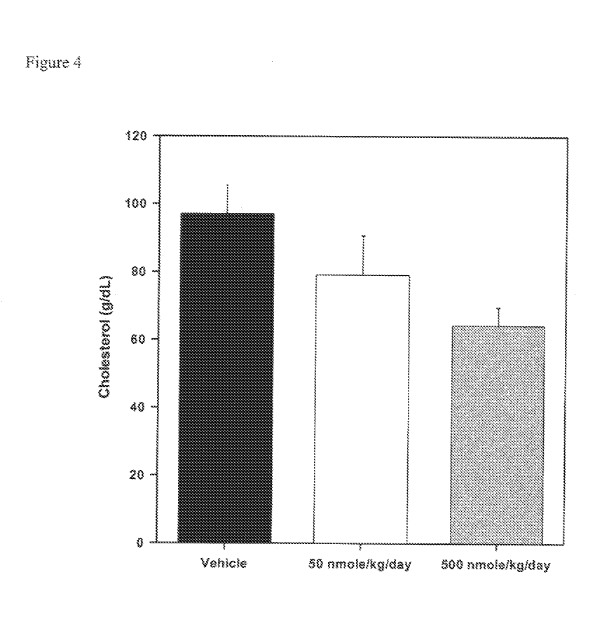

Treatment with 500 nmole/kg/day of Compound A decreased basal fasted plasma glucose. Both the low and the high doses of Compound A decreased plasma glucose levels after a glucose challenge (FIG. 3). As shown in FIG. 4, a 7 day infusion of Compound A decreased the amount of insulin required to clear the glucose challenge of the glucose tolerance test.

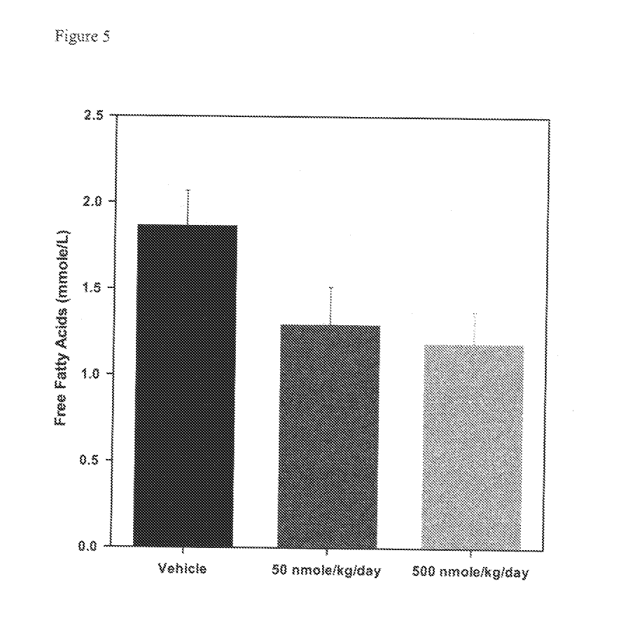

FIGS. 5, 6 and 7 show the dose-related decreases in triglycerides, cholesterol and free fatty acids which resulted from treatment with Compound A.

### Histology Studies

Progression of AFLD and NAFLD through the various stages of fat accumulation (in both micro and macro vesicles), cell death, lobular inflammation, ballooning degeneration, cirrhosis, formation of tumors and cancers, inflammation, and fibrosis may be monitored via tissue examination.

Development and progression of AFLD and NAFLD may be made at the gross tissue level. Livers, in situ or dissected from control or test animals, are inspected for appearance, weight, color, odor and other visually observable characteristics at various stages prior to and following administration of Compound A or vehicle control.

Development and progression of AFLD and NAFLD may also be made at the microscopic tissue level. Liver tissues are extracted from test and control animals at various stages prior to and following administration of Compound A. The tissues are flash frozen or otherwise preserved for fixation, microtoming and staining procedures, and the like.

The skilled artisan would know and appreciate that a variety of methods are available to study the tissue samples (see for example Matteoni C A et al. Gastroenterology 116:1413, 1999) and that a variety of visualization aids may be employed such as, but not limited to, oil red staining, immunohistochemical staining for visualization of macrophages, fibroblasts, collage and the like, NAFLD activity scores, AFLD activity scores, visualization of glutamine synthesis, detection of eosinophic cells, detection of HE or endogenous ALP, appearance of infiltrating cells, incorporation of BrdU to measure cellular proliferation, hematoxylin-eosin staining, Masson's trichome staining and gene expression analysis for tumor necrosis factors such as TNF-α, for interleukins such as IL-6 or IL-10 and for interferons such as IFN-γ and the like.

### Administration and Use

The peptides of this invention can be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic, or pamoic acid), inorganic acids (e.g., hydrochloric acid, sulfuric acid, or phosphoric acid), and polymeric acids (e.g., tannic acid, carboxymethyl cellulose, polylactic, polyglycolic, or copolymers of polylactic-glycolic acids). A typical method of making a salt of a peptide of the present invention is well known in the art and can be accomplished by standard methods of salt exchange. Accordingly, the TFA salt of a peptide of the present invention (the TFA salt results from the purification of the peptide by using preparative HPLC, eluting with TFA containing buffer solutions) can be converted into another salt, such as an acetate salt, by dissolving the peptide in a small amount of 0.25 N acetic acid aqueous solution. The resulting solution is applied to a semi-prep HPLC column (Zorbax®, 300 SB, C-8). The column is eluted with: (1) 0.1N ammonium acetate aqueous solution for 0.5 hours; (2) 0.25N acetic acid aqueous solution for 0.5 hours; and (3) a linear gradient (20% to 100% of solution B over 30 minutes) at a flow rate of 4 ml/min (solution A is 0.25N acetic acid aqueous solution; solution B is 0.25N acetic acid in acetonitrile/water, 80:20). The fractions containing the peptide are collected and lyophilized to dryness.

As is well known to those skilled in the art, the known and potential uses of peptides with melanocortin receptor (MC-R) agonist or antagonist activity is varied and multitudinous, thus the administration of the compounds of this invention for purposes of eliciting an agonist effect can have the same effects and uses as melanocortin itself.

Accordingly, the present invention includes within its scope pharmaceutical compositions comprising, as an active ingredient, at least one of the compounds of formula (I) in association with a pharmaceutically acceptable carrier.

The dosage of active ingredient in the compositions of this invention may be varied; however, it is necessary that the amount of the active ingredient be such that a suitable dosage form is obtained. The selected dosage depends upon the desired therapeutic effect, on the route of administration, and on the duration of the treatment. In general, an effective dosage for the activities of this invention is in the range of 1×10⁻⁷ to 200 mg/kg/day, preferably 1×10⁻⁴ to 100 mg/kg/day which can be administered as a single dose or divided into multiple doses.

The compounds of this invention can be administered by oral, parenteral (e.g., intramuscular, intraperitoneal, intravenous or subcutaneous injection, or implant), nasal, vaginal, rectal, sublingual or topical routes of administration and can be formulated with pharmaceutically acceptable carriers to provide dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. Preparations may be sterilized by, for example, filtration through a bacteria-retaining filter, by incorporating sterilizing agents into the compositions, by irradiating the compositions, or by heating the compositions. Preparations can also be manufactured in the form of sterile solid compositions which can be dissolved in sterile water or some other sterile injectable medium immediately before use.

Compositions for rectal or vaginal administration are preferably suppositories which may contain, in addition to the active substance, excipients such as cocoa butter or a suppository wax.

Compositions for nasal or sublingual administration are also prepared with standard excipients well known in the art.

Further, a compound of this invention can be administered in a sustained release composition such as those described in the following patents and patent applications. U.S. Pat. No. 5,672,659 teaches sustained release compositions comprising a bioactive agent and a polyester. U.S. Pat. No. 5,595,760 teaches sustained release compositions comprising a bioactive agent in a gelable form. U.S. Pat. No. 5,821,221 teaches polymeric sustained release compositions comprising a bioactive agent and chitosan. U.S. Pat. No. 5,916,883 teaches sustained release compositions comprising a bioactive agent and cyclodextrin. The teachings of the foregoing patents and applications are incorporated herein by reference.

What is claimed is:

1. A method of treating dyslipidemia in a subject in need thereof, comprising peripheral administration of an effective amount of a melanocortin receptor 4 agonist to treat said dyslipidemia in said subject in need thereof.

2. A method according to claim 1, wherein said treatment of dyslipidemia results in decreased levels of serum cholesterol, triglycerides, low-density lipoprotein cholesterol, free fatty acids, or increased levels of high-density lipoprotein cholesterol, or any combination thereof.

3. A method according to claim 1 wherein said subject is suffering from hepatic steatosis.

4. A method according to claim 3, wherein said hepatic steatosis is non-alcoholic fatty acid liver disease or alcoholic fatty acid liver disease.

5. A method according to claim 4, wherein said non-alcoholic fatty acid liver disease or alcoholic fatty acid liver disease is accompanied by steatohepatitis, steatonecrosis, lobular inflammation, ballooning degeneration, fibrosis, cirrhosis or cancer or any combination thereof.

6. The method according to claim 1, wherein said melanocortin receptor 4 agonist useful to treat dyslipidemia is selected from the group consisting of:
Ac-Arg-c(Cys-D-Ala-His-D-Phe-Arg-Trp-Cys)-NH₂;
Ac-Nle-c(Asp-His-D-Phe-Arg-Trpβ-Ala-Lys)-NH₂;
Ac-Nle-c(Asp-His-D-Phe-Arg-Trp-A6c-Lys)-NH₂;
Ac-Nle-c(Cys-His-D-Phe-Arg-Trp-Ahx-Cys)-NH₂;
D-Phe-c(Cys-His-D-Phe-Arg-Trp-Ala-D-Cys)-Thr-NH₂;
D-Phe-c(Cys-His-D-Phe-Arg-Trp-β-Ala-D-Cys)-Thr-NH₂;
D-Phe-c(Cys-His-D-Phe-Arg-Trp-Gaba-D-Cys)-Thr-NH₂;
Ac-Nle-c(Cys-His-D-Phe-Arg-Trp-Apn-Cys)-NH₂;
Ac-Nle-c(Asp-His-D-Phe-Arg-Trp-Apn-Lys)-NH₂;
Ac-A6c-c(Asp-His-D-Phe-Arg-Trp-Gaba-Lys)-NH₂;
Ac-D-2-Nal-c(Asp-His-D-Phe-Arg-Trp-Gaba-Lys)-NH₂;
Ac-Cha-c(Asp-His-D-Phe-Arg-Trp-Gaba-Lys)-NH₂;
Ac-Nle-c(Asp-His-D-Phe-Arg-Trp-Gaba-Lys)-NH₂;
Ac-Nle-c(Cys-D-Ala-His-D-Phe-Arg-Trp-Cys)-NH₂;
Ac-Nle-c(Cys-β-Ala-His-D-Phe-Arg-Trp-Cys)-NH₂;
Ac-Nle-c(Cys-Gaba-His-D-Phe-Arg-Trp-Cys)-NH₂;
Ac-Nle-c(Cys-Aib-His-D-Phe-Arg-Trp-Cys)-NH₂;
Ac-Nle-c(Cys-Gly-His-D-Phe-Arg-Trp-Cys)-NH₂;
Ac-Nle-c(D-Cys-Ala-His-D-Phe-Arg-Trp-Cys)-NH₂;
Ac-Nle-c(D-Cys-D-Ala-His-D-Phe-Arg-Trp-Cys)-NH₂;
Ac-Nle-c(D-Cys-β-Ala-His-D-Phe-Arg-Trp-Cys)-NH₂;
Ac-Nle-c(D-Cys-Gaba-His-D-Phe-Arg-Trp-Cys)-NH₂;
Ac-Nle-c(D-Cys-Aib-His-D-Phe-Arg-Trp-Cys)-NH₂;
Ac-Nle-c(D-Cys-Gly-His-D-Phe-Arg-Trp-Cys)-NH₂;
Ac-Nle-c(Cys-D-Ala-His-D-Phe-Arg-Trp-D-Cys)-NH₂;
Ac-Nle-c(Cys-β-Ala-His-D-Phe-Arg-Trp-D-Cys)-NH₂;
Ac-Nle-c(Cys-Gaba-His-D-Phe-Arg-Trp-D-Cys)-NH₂;
Ac-Nle-c(Cys-Aib-His-D-Phe-Arg-Trp-D-Cys)-NH₂;
Ac-Nle-c(Cys-Gly-His-D-Phe-Arg-Trp-D-Cys)-NH₂;
Ac-Nle-c(D-Cys-Ala-His-D-Phe-Arg-Trp-D-Cys)-NH₂;
Ac-Nle-c(D-Cys-D-Ala-His-D-Phe-Arg-Trp-D-Cys)-NH₂;
Ac-Nle-c(D-Cys-β-Ala-His-D-Phe-Arg-Trp-D-Cys)-NH₂;
Ac-Nle-c(D-Cys-Gaba-His-D-Phe-Arg-Trp-D-Cys)-NH₂;
Ac-Nle-c(D-Cys-Aib-His-D-Phe-Arg-Trp-D-Cys)-NH₂;
Ac-Oic-c(Asp-His-D-Phe-Arg-Trp-Gaba-Lys)-NH₂;
Ac-Chg-c(Asp-His-D-Phe-Arg-Trp-Gaba-Lys)-NH₂;
Ac-hCha-c(Asp-His-D-Phe-Arg-Trp-Gaba-Lys)-NH₂;
Ac-D-Cha-c(Asp-His-D-Phe-Arg-Trp-Gaba-Lys)-NH₂;
Ac-Nip-c(Asp-His-D-Phe-Arg-Trp-Gaba-Lys)-NH₂;
Ac-hPro-c(Asp-His-D-Phe-Arg-Trp-Gaba-Lys)-NH₂;
Ac-hLeu-c(Asp-His-D-Phe-Arg-Trp-Gaba-Lys)-NH₂;
Ac-Phe-c(Asp-His-D-Phe-Arg-Trp-Gaba-Lys)-NH₂;
Ac-D-Phe-c(Asp-His-D-Phe-Arg-Trp-Gaba-Lys)-NH₂;
Ac-D-Chg-c(Asp-His-D-Phe-Arg-Trp-Gaba-Lys)-NH₂;
n-butanoyl-Cha-c(Asp-His-D-Phe-Arg-Trp-Gaba-Lys)-NH₂;
Ac-hPhe-c(Asp-His-D-Phe-Arg-Trp-Gaba-Lys)-NH₂;
Ac-β-hMet-c(Asp-His-D-Phe-Arg-Trp-Gaba-Lys)-NH₂;
Ac-Gaba-c(Asp-His-D-Phe-Arg-Trp-Gaba-Lys)-NH₂;
Ac-Cha-c(Asp-His-D-Phe-Arg-D-Trp-Ala-Lys)-NH₂;
Ac-hCha-c(Asp-His-D-Phe-Arg-D-Trp-Ala-Lys)-NH₂;
Ac-Leu-c(Asp-His-D-Phe-Arg-D-Trp-Ala-Lys)-NH₂;
Ac-hLeu-c(Asp-His-D-Phe-Arg-D-Trp-Ala-Lys)-NH₂;
Ac-Phe-c(Asp-His-D-Phe-Arg-D-Trp-Ala-Lys)-NH₂;
Ac-Nle-c(Asp-His-D-Phe-Arg-D-Trp-D-Ala-Lys)-NH₂;
Ac-Nle-c(Asp-His-D-Phe-Arg-D-Trp-β-Ala-Lys)-NH₂;
Ac-Nle-c(Asp-His-D-Phe-Arg-D-Trp-Gaba-Lys)-NH₂;
Ac-Nle-c(Asp-His-D-Phe-Arg-D-Trp-Aha-Lys)-NH₂;
Ac-Nle-c(Asp-His-D-Phe-Arg-D-Trp-Apn-Lys)-NH₂;
Ac-Nle-c(Cys-His-D-Phe-Arg-D-Trp-Apn-Cys)-NH₂;
Ac-Nle-c(Cys-His-D-Phe-Arg-D-Trp-Gaba-Cys)-NH₂;
Ac-Nle-c(Cys-His-D-Phe-Arg-D-Trp-Ahx-Cys)-NH₂;
Ac-Nle-c(Cys-His-D-Phe-Arg-D-Trp-β-Ala-Cys)-NH₂;
Ac-Nle-c(Cys-His-D-Phe-Arg-D-Trp-D-Ala-Cys)-NH₂;
Ac-Nle-c(Cys-D-Ala-His-D-2-Nal-Arg-Trp-Cys)-NH₂;
Ac-Nle-c(Cys-D-Ala-His-D-2-Nal-Arg-2-Nal-Cys)-NH₂;
Ac-Nle-c(Cys-D-Ala-His-D-2-Nal-Arg-1-Nal-Cys)-NH₂;
n-butanoyl-Nle-c(Cys-D-Ala-His-D-Phe-Arg-2-Nal-Cys)-NH₂;
n-butanoyl-Nle-c(Cys-D-Ala-His-D-Phe-Arg-Trp-Cys)-NH₂;
Ac-Nle-c(Cys-D-Ala-His-D-Phe-Arg-2-Nal-Cys)-NH₂;
Ac-Nle-c(Cys-D-Ala-His-D-Phe-Arg-1-Nal-Cys)-NH₂;
Ac-Nle-c(Cys-D-Ala-His-D-Phe-Arg-Bal-Cys)-NH₂;
Ac-Nle-c(Cys-D-Glu-His-D-Phe-Arg-Trp-Cys)-NH₂;
Ac-Nle-c(Asp-His-D-Phe-Arg-Trp-D-Ala-Lys)-NH₂;
Ac-Nle-c(Cys-D-Ala-His-D-2-Nal-Arg-Bal-Cys)-NH₂;
Ac-Nle-c(Pen-D-Ala-His-D-Phe-Arg-Trp-Cys)-NH₂;
Ac-Nle-c(Cys-D-Ala-His-D-Phe-Arg-Trp-Pen)-NH₂;
Ac-Nle-c(Pen-D-Ala-His-D-Phe-Arg-Trp-Pen)-NH₂;
D-Phe-c(Cys-His-D-Phe-hArg-Trp-β-Ala-D-Cys)-Thr-NH₂;
D-Phe-c(Cys-His-D-(Et)Tyr-Arg-Trp-β-Ala-D-Cys)-Thr-NH₂;
D-Phe-c(Cys-His-D-Phe-Arg-Bip-β-Ala-D-Cys)-Thr-NH₂;
D-Phe-c(Cys-His-D-(Et)Tyr-hArg-Trp-β-Ala-D-Cys)-Thr-NH₂;
D-Phe-c(Cys-His-D-Phe-hArg-Bip-β-Ala-D-Cys)-Thr-NH₂;
D-Phe-c(Cys-His-D-(Et)Tyr-hArg-Bip-β-Ala-D-Cys)-Thr-NH₂;
Nle-c(Cys-His-D-Phe-Arg-Trp-Apn-Cys)-NH₂;
Ac-Nle-c(Asp-D-Ala-His-D-Phe-Arg-Trp-Lys)-NH₂;
Ac-Nle-c(Asp-D-Ala-His-D-Phe-Arg-Bal-Lys)-NH₂;
Ac-Nle-c(Cys-D-Ala-His-D-Phe-Arg-Trp-Pen)-OH;
Ac-Nle-c(Cys-D-Abu-His-D-Phe-Arg-Trp-Cys)-NH₂;
Ac-Nle-c(Cys-D-Val-His-D-Phe-Arg-Trp-Cys)-NH₂;
Ac-Nle-c(Cys-D-Ile-His-D-Phe-Arg-Trp-Cys)-NH₂;
Ac-Nle-c(Cys-D-Leu-His-D-Phe-Arg-Trp-Cys)-NH₂;
Ac-Nle-c(Cys-D-Tle-His-D-Phe-Arg-Trp-Cys)-NH₂;
Ac-Nle-c(Cys-D-Cha-His-D-Phe-Arg-Trp-Cys)-NH₂;
Ac-Nle-c(Pen-His-D-Phe-Arg-Trp-Gaba-Cys)-NH₂;
Ac-Nle-c(Cys-His-D-Phe-Arg-Trp-Gaba-Pen)-NH₂;
Ac-Nle-c(Pen-His-D-Phe-Arg-Trp-Gaba-Pen)-NH₂;
Ac-Leu-c(Cys-His-D-Phe-Arg-Trp-Gaba-Cys)-NH₂;
Ac-Cha-c(Cys-His-D-Phe-Arg-Trp-Gaba-Cys)-NH₂;
Ac-Ile-c(Cys-His-D-Phe-Arg-Trp-Gaba-Cys)-NH₂;
Ac-Phe-c(Cys-His-D-Phe-Arg-Trp-Gaba-Cys)-NH₂;
Ac-Val-c(Cys-His-D-Phe-Arg-Trp-Gaba-Cys)-NH₂;
Ac-2-Nal-c(Cys-His-D-Phe-Arg-Trp-Gaba-Cys)-NH₂;
Nle-c(Cys-His-D-Phe-Arg-Trp-Gaba-Cys)-NH₂;
Phe-c(Cys-His-D-Phe-Arg-Trp-Gaba-Cys)-NH₂;
Ac-Nle-c(Cys-3-Pal-D-Phe-Arg-Trp-Gaba-Cys)-NH₂;
Ac-Nle-c(Cys-D-Ala-His-D-Phe-Arg-Trp-Cys)-OH;
Ac-Nle-c(Cys-His-Phe-Arg-D-Trp-Gaba-Cys)-NH₂;
Ac-Nle-c(Asp-His-D-2-Nal-Arg-Trp-Ala-Lys)-NH₂;
Ac-Nle-c(Asp-His-D-2-Nal-Arg-Trp-β-Ala-Lys)-NH₂;
Ac-Nle-c(Cys-His-D-2-Nal-Arg-Trp-Gaba-Cys)-NH₂;
Ac-Nle-c(Cys-His-D-2-Nal-Arg-Trp-Ahx-Cys)-NH₂;
Ac-hPhe-c(Asp-His-D-2-Nal-Arg-Trp-Gaba-Lys)-NH₂;
Ac-Cha-c(Asp-His-D-2-Nal-Arg-Trp-Gaba-Lys)-NH₂;
Ac-Nle-c(Asp-His-D-Phe-Arg-Trp-β-Ala-Lys)—OH;
Ac-Nle-c(Cys-His-D-Phe-Arg-Trp-Ahx-Cys)-OH;
D-Phe-c(Cys-His-D-Phe-Arg-Trp-Ala-D-Cys)-Thr-OH;
D-Phe-c(Cys-His-D-Phe-Arg-Trp-β-Ala-D-Cys)-Thr-OH;
D-Phe-c(Cys-His-D-Phe-Arg-Trp-Gaba-D-Cys)-Thr-OH;
Ac-Nle-c(Cys-His-D-Phe-Arg-Trp-Apn-Cys)-OH;
Ac-Nle-c(Asp-His-D-Phe-Arg-Trp-Apn-Lys)-OH;
Ac-Cha-c(Asp-His-D-Phe-Arg-Trp-Gaba-Lys)-OH;
Ac-Nle-c(Asp-His-D-Phe-Arg-Trp-Gaba-Lys)—OH;
Ac-Chg-c(Asp-His-D-Phe-Arg-Trp-Gaba-Lys)—OH;
Ac-D-Cha-c(Asp-His-D-Phe-Arg-Trp-Gaba-Lys)—OH;
Ac-hCha-c(Asp-His-D-Phe-Arg-Trp-Gaba-Lys)-OH;
Ac-D-Chg-c(Asp-His-D-Phe-Arg-Trp-Gaba-Lys)—OH;
Ac-hPhe-c(Asp-His-D-Phe-Arg-Trp-Gaba-Lys)—OH;
Ac-Nle-c(Cys-His-D-Phe-Arg-D-Trp-Gaba-Cys)-OH;
Ac-Nle-c(Cys-His-D-Phe-Arg-D-Trp-Ahx-Cys)-OH;
Ac-Nle-c(Cys-His-D-Phe-Arg-D-Trp-β-Ala-Cys)—OH;
Ac-Nle-c(Cys-His-D-Phe-Arg-D-Trp-D-Ala-Cys)-OH;
Ac-Nle-c(Cys-D-Ala-His-D-2-Nal-Arg-Trp-Cys)-OH;
Ac-Nle-c(Cys-D-Ala-His-D-2-Nal-Arg-2-Nal-Cys)-OH;
Ac-Nle-c(Cys-D-Ala-His-D-2-Nal-Arg-1-Nal-Cys)-OH;
Ac-Nle-c(Cys-D-Ala-His-D-2-Nal-Arg-Bal-Cys)-OH;
Ac-Nle-c(Pen-D-Ala-His-D-Phe-Arg-Trp-Cys)-OH;
Ac-Nle-c(Cys-His-D-Phe-Arg-Trp-Gaba-Pen)—OH;
Ac-Arg-c(Cys-D-Ala-His-D-2-Nal-Arg-Trp-Cys)-NH₂;
Ac-D-Arg-c(Cys-D-Ala-His-D-Phe-Arg-Trp-Cys)-NH₂;
Ac-D-Arg-c(Cys-D-Ala-His-D-Phe-Arg-Trp-Pen)-NH₂;
Ac-D-Arg-c(Cys-His-D-Phe-Arg-Trp-Gaba-Pen)-NH₂;
Ac-Arg-c(Cys-His-D-Phe-Arg-Trp-Gaba-Pen)-NH₂;
Ac-Arg-c(Cys-D-Ala-His-D-Phe-Arg-Trp-Pen)-NH₂;
Ac-D-Arg-c(Asp-His-D-Phe-Arg-Trp-Ala-Lys)-NH₂;
Ac-Arg-c(Asp-His-D-Phe-Arg-Trp-Ala-Lys)-NH₂;
Ac-Nle-c(Cys-3-Pal-D-Phe-Arg-Trp-Gaba-Cys)-NH₂;
Ac-Nle-c(Cys-D-Ala-His-D-Phe-Arg-Trp-Gly-Cys)-NH₂;
Ac-Nle-c(Cys-D-Ala-His-D-Phe-Arg-Trp-D-Ala-Cys)-NH₂;
Ac-Nle-c(Cys-D-Ala-His-D-Phe-Arg-Trp-β-Ala-Cys)-NH₂;
Ac-Nle-c(Cys-D-Ala-His-D-Phe-Arg-Trp-Gaba-Cys)-NH₂;
Ac-Nle-c(Cys-D-Ala-His-D-Phe-Arg-Trp-Apn-Cys)-NH₂;
Ac-c(Cys-Glu-His-D-Phe-Arg-Trp-Ala-Cys)-NH₂;
Ac-c(Cys-Glu-His-D-Phe-Arg-2-Nal-Ala-Cys)-NH₂;
Ac-c(Cys-D-Ala-His-D-Phe-Arg-Trp-Ala-Cys)-NH₂;
Ac-c(Cys-D-Ala-His-D-Phe-Arg-2-Nal-Ala-Cys)-NH₂;
Ac-Nle-c(Cys-D-Ala-His-D-Phe-Arg-Trp-Ala-Cys)-NH₂;
Ac-Nle-c(Cys-D-Ala-His-D-Phe-Arg-Trp-β-Ala-Cys)-NH₂;
Ac-Nle-c(Asp-D-Ala-His-D-Phe-Arg-Bal-Ala-Lys)-NH₂;
Tyr-Gly-Arg-(Lys)₂-(Arg)₂-Gln-(Arg)₃-Nle-c(Asp-His-D-2-Nal-Arg-Trp-Lys)-NH₂;
Tyr-Gly-Arg-(Lys)₂-(Arg)₂-Gln-(Arg)₃-Doc-Nle-c(Asp-His-D-2-Nal-Arg-Trp-Lys)-NH₂;
Nle-c(Asp-His-D-2-Nal-Arg-Trp-Lys)-β-Ala-Tyr-Gly-Arg-(Lys)₂-(Arg)₂-Gln-(Arg)₃-NH₂;
Ac-Nle-c(Asp-His-D-2-Nal-Arg-Trp-Lys)-β-Ala-Tyr-Gly-Arg-(Lys)₂-(Arg)₂-Gln-(Arg)₃-NH₂;
Nle-c(Asp-His-D-2-Nal-Arg-Trp-Lys)-(Doc)₂-Tyr-Gly-Arg-(Lys)₂-(Arg)₂-Gln-(Arg)₃-NH₂;
Ac-Nle-c(Asp-His-D-2-Nal-Arg-Trp-Lys)-(Pro)₂-Lys-Asp-Tyr-Gly-Arg-(Lys)₂-(Arg)₂-Gln-(Arg)₃-NH₂;
Ac-c(Cys-Glu-His-D-2-Nal-Arg-Trp-Gly-Cys)-(Pro)₂-Lys-Asp-Tyr-Gly-Arg-(Lys)₂-(Arg)₂-Gln-(Arg)₃-NH₂;
Ac-Nle-c(Asp-His-D-2-Nal-Arg-Trp-Lys)-(β-Ala)₂-Tyr-Gly-Arg-(Lys)₂-(Arg)₂-Gln-(Arg)₃-NH₂;
Ac-Nle-c(Asp-His-D-2-Nal-Arg-Trp-Lys)-(Pro)₂-Lys-Asp-Doc-Tyr-Gly-Arg-(Lys)₂-(Arg)₂-Gln-(Arg)₃-NH₂;
Ac-c(Cys-Glu-His-D-2-Nal-Arg-Trp-Gly-Cys)-(Pro)₂-Lys-Asp-Doc-Tyr-Gly-Arg-(Lys)₂-(Arg)₂-Gln-(Arg)₃-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₂-Gln-(Arg)₃-NH₂;
Ac-c(Cys-Glu-His-D-2-Nal-Arg-Trp-Ala-Cys)-(Pro)₂-Lys-Asp-Doc-Tyr-Gly-Arg-(Lys)₂-(Arg)₂-Gln-(Arg)₃-NH₂;
Ac-Nle-c(Asp-His-D-2-Nal-Arg-Trp-Lys)-(Doc)₂-Tyr-Gly-Arg-(Lys)₂-(Arg)₂-Gln-(Arg)₃-NH₂;
Ac-c(Cys-Glu-His-D-2-Nal-Arg-2-Nal-Ala-Cys)-(Pro)₂-Lys-Asp-β-Ala-Tyr-Gly-Arg-(Lys)₂-(Arg)₂-Gln-(Arg)₃-NH₂;
Ac-c(Cys-Glu-His-D-2-Nal-Arg-Bal-Ala-Cys)-(Pro)₂-Lys-Asp-β-Ala-(Arg)₅-Gln-(Arg)₃-NH₂;
Ac-c(Cys-Glu-His-D-2-Nal-Arg-Bal-Ala-Cys)-(Pro)₂-Lys-Asp-β-Ala-Gly-(Arg)₅-Gln-(Arg)₃-NH₂;
Ac-c(Cys-Glu-His-D-2-Nal-Arg-Bal-Ala-Cys)-(Pro)₂-Lys-Asp-β-Ala-Tyr-Gly-(Arg)₅-Gln-(Arg)₃-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-Arg-Gln-(Arg)₄-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-Gln-(Arg)₅-NH₂;
Ac-c(Cys-Glu-His-D-2-Nal-Arg-Trp-Ala-Cys)-(Pro)₂-Lys-Asp-β-Ala-Tyr-Gly-Arg-Lys-Gln-Lys-(Arg)₅-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₄-Gln-Arg-NH₂;
Ac-c(Cys-Glu-His-D-2-Nal-Arg-Bal-Ala-Cys)-(Pro)₂-Lys-Asp-β-Ala-Tyr-Aib-Arg-(Lys)₂-(Arg)₂-Gln-(Arg)₃-NH₂;
Ac-c(Cys-Glu-His-D-2-Nal-Arg-1-Nal-Ala-Cys)-(Pro)₂-Arg-Asp-β-Ala-(Arg)₅-Gln-(Arg)₃-NH₂;
Ac-c(Cys-Glu-His-D-2-Nal-Arg-1-Nal-Ala-Cys)-(Pro)₂-Lys-Asp-β-Ala-(Arg)₅-Gln-(Arg)₃-NH₂;
Ac-c(Cys-Glu-His-D-2-Nal-Arg-1-Nal-Ala-Cys)-(Pro)₂-Lys-Asp-β-Ala-(Arg)₆-Gln-(Arg)₃-NH₂;
Ac-c(Cys-Glu-His-D-2-Nal-Arg-2-Nal-Ala-Cys)-(Pro)₂-Arg-Asp-β-Ala-(Arg)₅-Gln-(Arg)₃-NH₂;
Ac-c(Cys-Glu-His-D-2-Nal-Arg-2-Nal-Ala-Cys)-(Pro)₂-Lys-Asp-β-Ala-(Arg)₅-Gln-(Arg)₃-NH₂;
Ac-c(Cys-Glu-His-D-2-Nal-Arg-2-Nal-Ala-Cys)-(Pro)₂-Lys-Asp-β-Ala-(Arg)₆-Gln-(Arg)₃-NH₂;
Ac-c(Cys-Glu-His-D-2-Nal-Arg-Bal-Ala-Cys)-(Pro)₂-Arg-Asp-β-Ala-(Arg)₆-Gln-(Arg)₃-NH₂;
Ac-c(Cys-Glu-His-D-2-Nal-Arg-Bal-Ala-Cys)-(Pro)₂-Arg-Asp-β-Ala-(Arg)₅-Gln-(Arg)₃-NH₂;
Ac-c(Cys-Glu-His-D-2-Nal-Arg-Bal-Ala-Cys)-(Pro)₂-Lys-Asp-β-Ala-(Arg)₆-Gln-(Arg)₃-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₃-Gln-(Arg)₂-NH₂;
Ac-c(Cys-Glu-His-D-2-Nal-Arg-Trp-Ala-Cys)-(Pro)₂-Lys-Asp-β-Ala-Tyr-Gly-Arg-Gln-(Lys)₂-(Arg)₅-NH₂;
Ac-c(Cys-Glu-His-D-2-Nal-Arg-Trp-Ala-Cys)-(Pro)₂-Lys-Asp-β-Ala-Tyr-Gly-Arg-(Lys)₂-(Arg)₅-Gln-NH₂;
Ac-c(Cys-Glu-His-D-2-Nal-Arg-1-Nal-Ala-Cys)-(Pro)₂-Lys-Asp-β-Ala-Tyr-Gly-Arg-(Lys)₂-(Arg)₂-Gln-(Arg)₃-NH₂;
Ac-c(Cys-Glu-His-D-2-Nal-Arg-Bal-Ala-Cys)-(Pro)₂-Lys-Asp-β-Ala-Tyr-Gly-Arg-(Lys)₂-(Arg)₂-Gln-(Arg)₃-NH₂;
Ac-c(Cys-Glu-His-D-2-Nal-Arg-1-Nal-Ala-Cys)-(Pro)₂-Lys-Asp-β-Ala-(Arg)₂-Lys-(Arg)₂-Gln-(Arg)₃-NH₂;
Ac-c(Cys-Glu-His-D-2-Nal-Arg-1-Nal-Ala-Cys)-(Pro)₂-Lys-Asp-β-Ala-Arg-Lys-(Arg)₃-Gln-(Arg)₃-NH₂;
Ac-c(Cys-Glu-His-D-2-Nal-Arg-2-Nal-Ala-Cys)-(Pro)₂-Lys-Asp-β-Ala-(Arg)₂-Lys-(Arg)₂-Gln-(Arg)₃-NH₂;
Ac-c(Cys-Glu-His-D-2-Nal-Arg-2-Nal-Ala-Cys)-(Pro)₂-Lys-Asp-β-Ala-Tyr-Gly-(Arg)₂-Lys-(Arg)₂-Gln-(Arg)₃-NH₂;
Ac-c(Cys-Glu-His-D-2-Nal-Arg-2-Nal-Ala-Cys)-(Pro)₂-Lys-Asp-β-Ala-Gly-(Arg)₂-Lys-(Arg)₂-Gln-(Arg)₃-NH₂;
Ac-c(Cys-Glu-His-D-2-Nal-Arg-2-Nal-Ala-Cys)-(Pro)₂-Lys-Asp-β-Ala-Gly-Arg-Lys-(Arg)₃-Gln-(Arg)₃-NH₂;
Ac-c(Cys-Glu-His-D-2-Nal-Arg-1-Nal-Ala-Cys)-(Pro)₂-Lys-Asp-β-Ala-Tyr-Gly-(Arg)₂-Lys-(Arg)₂-Gln-(Arg)₃-NH₂;
Ac-c(Cys-Glu-His-D-2-Nal-Arg-1-Nal-Ala-Cys)-(Pro)₂-Lys-Asp-β-Ala-Tyr-Gly-Arg-Lys-(Arg)₃-Gln-(Arg)₃-NH₂;
Ac-c(Cys-Glu-His-D-2-Nal-Arg-1-Nal-Ala-Cys)-(Pro)₂-Lys-Asp-β-Ala-Gly-(Arg)₂-Lys-(Arg)₂-Gln-(Arg)₃-NH₂;
Ac-c(Cys-Glu-His-D-2-Nal-Arg-1-Nal-Ala-Cys)-(Pro)₂-Lys-Asp-β-Ala-Gly-Arg-Lys-(Arg)₃-Gln-(Arg)₃-NH₂;
Ac-c(Cys-Glu-His-D-2-Nal-Arg-2-Nal-Ala-Cys)-(Pro)₂-Lys-Asp-β-Ala-(Arg)₂-Lys-(Arg)₂-Gln-(Arg)₃-NH₂;
Ac-c(Cys-Glu-His-D-2-Nal-Arg-2-Nal-Ala-Cys)-(Pro)₂-Lys-Asp-β-Ala-Arg-Lys-(Arg)₃-Gln-(Arg)₃-NH₂;
Ac-c(Cys-Glu-His-D-2-Nal-Arg-2-Nal-Ala-Cys)-(Pro)₂-Lys-Asp-β-Ala-Tyr-Gly-Arg-Lys-(Arg)₃-Gln-(Arg)₃-NH₂;
Ac-c(Cys-Glu-His-D-2-Nal-Arg-Bal-Ala-Cys)-(Pro)₂-Lys-Asp-β-Ala-(Arg)₂-Lys-(Arg)₂-Gln-(Arg)₃-NH₂;
Ac-c(Cys-Glu-His-D-2-Nal-Arg-Bal-Ala-Cys)-(Pro)₂-Lys-Asp-β-Ala-Arg-Lys-(Arg)₃-Gln-(Arg)₃-NH₂;
Ac-c(Cys-Glu-His-D-2-Nal-Arg-Bal-Ala-Cys)-(Pro)₂-Lys-Asp-β-Ala-Tyr-Gly-(Arg)₂-Lys-(Arg)₂-Gln-(Arg)₃-NH₂;
Ac-c(Cys-Glu-His-D-2-Nal-Arg-Bal-Ala-Cys)-(Pro)₂-Lys-Asp-β-Ala-Tyr-Gly-Arg-Lys-(Arg)₃-Gln-(Arg)₃-NH₂;
Ac-c(Cys-Glu-His-D-2-Nal-Arg-Bal-Ala-Cys)-(Pro)₂-Lys-Asp-β-Ala-Gly-(Arg)₂-Lys-(Arg)₂-Gln-(Arg)₃-NH₂;
Ac-c(Cys-Glu-His-D-2-Nal-Arg-Bal-Ala-Cys)-(Pro)₂-Lys-Asp-β-Ala-Gly-Arg-Lys-(Arg)₃-Gln-(Arg)₃-NH₂;
Ac-c(Cys-Glu-His-D-2-Nal-Arg-Trp-Ala-Cys)-(Pro)₂-Lys-Asp-β-Ala-(Arg)₅-Gln-(Arg)₃-NH₂;
Ac-c(Cys-Glu-His-D-2-Nal-Arg-Trp-Ala-Cys)-(Pro)₂-Arg-Asp-β-Ala-(Arg)₅-Gln-(Arg)₃-NH₂;
Ac-c(Cys-Glu-His-D-2-Nal-Arg-Trp-Ala-Cys)-(Pro)₂-Lys-Asp-β-Ala-Tyr-Gly-(Arg)₅-Gln-(Arg)₃-NH₂;
Ac-c(Cys-Glu-His-D-2-Nal-Arg-Trp-Ala-Cys)-(Pro)₂-Arg-Asp-β-Ala-Tyr-Gly-(Arg)₅-Gln-(Arg)₃-NH₂;
Ac-c(Cys-Glu-His-D-2-Nal-Arg-Trp-Ala-Cys)-(Pro)₂-Lys-Asp-β-Ala-(Arg)₅-Gln-(Arg)₄-NH₂;
Ac-c(Cys-Glu-His-D-2-Nal-Arg-Trp-Ala-Cys)-(Pro)₂-Arg-Asp-β-Ala-(Arg)₅-Gln-(Arg)₄-NH₂;
Ac-c(Cys-Glu-His-D-2-Nal-Arg-Trp-Ala-Cys)-(Pro)₂-Lys-Asp-β-Ala-Tyr-Gly-(Arg)₅-Gln-(Arg)₄-NH₂;
Ac-c(Cys-Glu-His-D-2-Nal-Arg-Trp-Ala-Cys)-(Pro)₂-Arg-Asp-β-Ala-Tyr-Gly-(Arg)₅-Gln-(Arg)₄-NH₂;
Ac-c(Cys-Glu-His-D-2-Nal-Arg-1-Nal-Ala-Cys)-(Pro)₂-Lys-Asp-β-Ala-(Arg)₅-Gln-(Arg)₄-NH₂;
Ac-c(Cys-Glu-His-D-2-Nal-Arg-1-Nal-Ala-Cys)-(Pro)₂-Arg-Asp-β-Ala-(Arg)₅-Gln-(Arg)₄-NH₂;
Ac-c(Cys-Glu-His-D-2-Nal-Arg-1-Nal-Ala-Cys)-(Pro)₂-Arg-Asp-β-Ala-(Arg)₆-Gln-(Arg)₃-NH₂;
Ac-c(Cys-Glu-His-D-2-Nal-Arg-1-Nal-Ala-Cys)-(Pro)₂-Lys-Asp-β-Ala-Tyr-Gly-(Arg)₅-Gln-(Arg)₃-NH₂;
Ac-c(Cys-Glu-His-D-2-Nal-Arg-1-Nal-Ala-Cys)-(Pro)₂-Arg-Asp-β-Ala-Tyr-Gly-(Arg)₅-Gln-(Arg)₃-NH₂;
Ac-c(Cys-Glu-His-D-2-Nal-Arg-1-Nal-Ala-Cys)-(Pro)₂-Lys-Asp-β-Ala-Tyr-Gly-(Arg)₅-Gln-(Arg)₄-NH₂;
Ac-c(Cys-Glu-His-D-2-Nal-Arg-1-Nal-Ala-Cys)-(Pro)₂-Arg-Asp-β-Ala-Tyr-Gly-(Arg)₅-Gln-(Arg)₄-NH₂;
Ac-c(Cys-Glu-His-D-2-Nal-Arg-1-Nal-Ala-Cys)-(Pro)₂-Lys-Asp-β-Ala-Tyr-Gly-(Arg)₆-Gln-(Arg)₃-NH₂;
Ac-c(Cys-Glu-His-D-2-Nal-Arg-1-Nal-Ala-Cys)-(Pro)₂-Arg-Asp-β-Ala-Tyr-Gly-(Arg)₆-Gln-(Arg)₃-NH₂;
Ac-c(Cys-Glu-His-D-2-Nal-Arg-2-Nal-Ala-Cys)-(Pro)₂-Arg-Asp-β-Ala-(Arg)₆-Gln-(Arg)₃-NH₂;
Ac-c(Cys-Glu-His-D-2-Nal-Arg-2-Nal-Ala-Cys)-(Pro)₂-Lys-Asp-β-Ala-(Arg)₅-Gln-(Arg)₄-NH₂;
Ac-c(Cys-Glu-His-D-2-Nal-Arg-2-Nal-Ala-Cys)-(Pro)₂-Arg-Asp-β-Ala-(Arg)₅-Gln-(Arg)₄-NH₂;
Ac-c(Cys-Glu-His-D-2-Nal-Arg-2-Nal-Ala-Cys)-(Pro)₂-Lys-Asp-β-Ala-Tyr-Gly-(Arg)₅-Gln-(Arg)₃-NH₂;
Ac-c(Cys-Glu-His-D-2-Nal-Arg-2-Nal-Ala-Cys)-(Pro)₂-Arg-Asp-β-Ala-Tyr-Gly-(Arg)₅-Gln-(Arg)₃-NH₂;
Ac-c(Cys-Glu-His-D-2-Nal-Arg-2-Nal-Ala-Cys)-(Pro)₂-Lys-Asp-β-Ala-Tyr-Gly-(Arg)₆-Gln-(Arg)₃-NH₂;
Ac-c(Cys-Glu-His-D-2-Nal-Arg-2-Nal-Ala-Cys)-(Pro)₂-Arg-Asp-β-Ala-Tyr-Gly-(Arg)₆-Gln-(Arg)₃-NH₂;
Ac-c(Cys-Glu-His-D-2-Nal-Arg-2-Nal-Ala-Cys)-(Pro)₂-Lys-Asp-β-Ala-Tyr-Gly-(Arg)₅-Gln-(Arg)₄-NH₂;
Ac-c(Cys-Glu-His-D-2-Nal-Arg-2-Nal-Ala-Cys)-(Pro)₂-Arg-Asp-β-Ala-Tyr-Gly-(Arg)₅-Gln-(Arg)₄-NH₂;
Ac-c(Cys-Glu-His-D-2-Nal-Arg-Bal-Ala-Cys)-(Pro)₂-Lys-Asp-β-Ala-(Arg)₅-Gln-(Arg)₄-NH₂;
Ac-c(Cys-Glu-His-D-2-Nal-Arg-Bal-Ala-Cys)-(Pro)₂-Arg-Asp-β-Ala-(Arg)₅-Gln-(Arg)₄-NH₂;
Ac-c(Cys-Glu-His-D-2-Nal-Arg-Bal-Ala-Cys)-(Pro)₂-Arg-Asp-β-Ala-Tyr-Gly-(Arg)₅-Gln-(Arg)₃-NH₂;
Ac-c(Cys-Glu-His-D-2-Nal-Arg-Bal-Ala-Cys)-(Pro)₂-Lys-Asp-β-Ala-Tyr-Gly-(Arg)₅-Gln-(Arg)₄-NH₂;
Ac-c(Cys-Glu-His-D-2-Nal-Arg-Bal-Ala-Cys)-(Pro)₂-Arg-Asp-β-Ala-Tyr-Gly-(Arg)₅-Gln-(Arg)₄-NH₂;
Ac-c(Cys-Glu-His-D-2-Nal-Arg-Bal-Ala-Cys)-(Pro)₂-Lys-Asp-β-Ala-Tyr-Gly-(Arg)₆-Gln-(Arg)₃-NH₂;
Ac-c(Cys-Glu-His-D-2-Nal-Arg-Bal-Ala-Cys)-(Pro)₂-Arg-Asp-β-Ala-Tyr-Gly-(Arg)₆-Gln-(Arg)₃-NH₂;
Ac-Nle-c(Cys-D-Ala-His-D-Phe-Arg-Trp-Cys)-(Doc)₂-Tyr-Gly-Arg-(Lys)₂-(Arg)₂-Gln-(Arg)₃-NH₂;
Ac-Nle-c(Cys-D-Ala-His-D-Phe-Arg-Trp-Cys)-β-Ala-Tyr-Gly-Arg-(Lys)₂-Arg-Gln-(Arg)₄-NH₂;
Ac-Nle-c(Cys-D-Ala-His-D-Phe-Arg-Trp-Cys)-Doc-Tyr-Gly-Arg-(Lys)₂-(Arg)₂-Gln-(Arg)₃-NH₂;
Ac-Nle-c(Cys-D-Ala-His-D-Phe-Arg-Trp-Cys)-β-Ala-(Arg)₅-Gln-(Arg)₃-NH₂;
Ac-Nle-c(Cys-D-Ala-His-D-Phe-Arg-Trp-Cys)-β-Ala-Gly-(Arg)₅-Gln-(Arg)₃-NH₂;
Ac-Nle-c(Cys-D-Ala-His-D-Phe-Arg-Trp-Cys)-β-Ala-Tyr-Gly-(Arg)₅-Gln-(Arg)₃-NH₂;
Ac-Nle-c(Cys-D-Ala-His-D-Phe-Arg-Trp-Cys)-β-Ala-Gly-(Arg)₅-Gln-(Arg)₄-NH₂;
Ac-Nle-c(Cys-D-Ala-His-D-Phe-Arg-Trp-Cys)-β-Ala-Tyr-Gly-(Arg)₂-Lys-(Arg)₂-Gln-(Arg)₃-NH₂;
Ac-Nle-c(Cys-D-Ala-His-D-Phe-Arg-Trp-Cys)-β-Ala-Tyr-Gly-Arg-Lys-(Arg)₃-Gln-(Arg)₃-NH₂;
Ac-Nle-c(Cys-D-Ala-His-D-Phe-Arg-Trp-Cys)-β-Ala-Gly-(Arg)₂-Lys-(Arg)₂-Gln-(Arg)₃-NH₂;
Ac-Nle-c(Cys-D-Ala-His-D-Phe-Arg-Trp-Cys)-β-Ala-Gly-Arg-Lys-(Arg)₃-Gln-(Arg)₃-NH₂;
Ac-Nle-c(Cys-D-Ala-His-D-Phe-Arg-Trp-Cys)-β-Ala-(Arg)₂-Lys-(Arg)₂-Gln-(Arg)₃-NH₂;
Ac-Nle-c(Cys-D-Ala-His-D-Phe-Arg-Trp-Cys)-β-Ala-Arg-Lys-(Arg)₃-Gln-(Arg)₃-NH₂;
Ac-Nle-c(Cys-D-Ala-His-D-Phe-Arg-Trp-Cys)-β-Ala-(Arg)₅-Gln-(Arg)₃-NH₂;
Ac-Nle-c(Cys-D-Ala-His-D-Phe-Arg-Trp-Cys)-(β-Ala)₂-(Arg)₅-Gln-(Arg)₃-NH₂;
Ac-Nle-c(Cys-D-Ala-His-D-Phe-Arg-Trp-Cys)-(β-Ala)₂-Gly-(Arg)₅-Gln-(Arg)₃-NH₂;
Ac-Nle-c(Cys-D-Ala-His-D-Phe-Arg-Trp-Cys)-(β-Ala)₂-Tyr-Gly-(Arg)₅-Gln-(Arg)₃-NH₂;
Ac-Nle-c(Cys-D-Ala-His-D-Phe-Arg-Trp-Cys)-Doc-(Arg)₅-Gln-(Arg)₃-NH₂;
Ac-Nle-c(Cys-D-Ala-His-D-Phe-Arg-Trp-Cys)-Doc-Gly-(Arg)₅-Gln-(Arg)₃-NH₂;
Ac-Nle-c(Cys-D-Ala-His-D-Phe-Arg-Trp-Cys)-Doc-Tyr-Gly-(Arg)₅-Gln-(Arg)₃-NH₂;
Ac-Nle-c(Cys-D-Ala-His-D-Phe-Arg-Trp-Cys)-(Doc)₂-(Arg)₅-Gln-(Arg)₃-NH₂;
Ac-Nle-c(Cys-D-Ala-His-D-Phe-Arg-Trp-Cys)-(Doc)₂-Gly-(Arg)₅-Gln-(Arg)₃-NH₂;
Ac-Nle-c(Cys-D-Ala-His-D-Phe-Arg-Trp-Cys)-(Doc)₂-Tyr-Gly-(Arg)₅-Gln-(Arg)₃-NH₂;
Ac-Nle-c(Cys-D-Ala-His-D-Phe-Arg-Trp-Cys)-β-Ala-(Arg)₅-Gln-(Arg)₄-NH₂;
Ac-Nle-c(Cys-D-Ala-His-D-Phe-Arg-Trp-Cys)-β-Ala-Tyr-Gly-(Arg)₅-Gln-(Arg)₄-NH₂;
Ac-Nle-c(Cys-D-Ala-His-D-Phe-Arg-Trp-Cys)-(β-Ala)₂-(Arg)₅-Gln-(Arg)₄-NH₂;
Ac-Nle-c(Cys-D-Ala-His-D-Phe-Arg-Trp-Cys)-(β-Ala)₂-Gly-(Arg)₅-Gln-(Arg)₄-NH₂;
Ac-Nle-c(Cys-D-Ala-His-D-Phe-Arg-Trp-Cys)-(β-Ala)₂-Tyr-Gly-(Arg)₅-Gln-(Arg)₄-NH₂;
Ac-Nle-c(Cys-D-Ala-His-D-Phe-Arg-Trp-Cys)-Doc-(Arg)₅-Gln-(Arg)₄-NH₂;
Ac-Nle-c(Cys-D-Ala-His-D-Phe-Arg-Trp-Cys)-Doc-Gly-(Arg)₅-Gln-(Arg)₄-NH₂;
Ac-Nle-c(Cys-D-Ala-His-D-Phe-Arg-Trp-Cys)-Doc-Tyr-Gly-(Arg)₅-Gln-(Arg)₄-NH₂;
Ac-Nle-c(Cys-D-Ala-His-D-Phe-Arg-Trp-Cys)-(Doc)₂-(Arg)₅-Gln-(Arg)₄-NH₂;
Ac-Nle-c(Cys-D-Ala-His-D-Phe-Arg-Trp-Cys)-(Doc)₂-Gly-(Arg)₅-Gln-(Arg)₄-NH₂;
Ac-Nle-c(Cys-D-Ala-His-D-Phe-Arg-Trp-Cys)-(Doc)₂-Tyr-Gly-(Arg)₅-Gln-(Arg)₄-NH₂;
Ac-Nle-c(Cys-His-D-Phe-Arg-Trp-Gaba-Cys)-β-Ala-Tyr-Gly-(Arg)₅-Gln-(Arg)₃-NH₂;
Ac-Nle-c(Cys-His-D-Phe-Arg-Trp-Gaba-Cys)-β-Ala-(Arg)₅-Gln-(Arg)₃-NH₂;
Ac-Nle-c(Asp-His-D-Phe-Arg-Trp-Ala-Lys)-β-Ala-Tyr-Gly-(Arg)₅-Gln-(Arg)₃-NH₂;
Ac-Nle-c(Asp-His-D-Phe-Arg-Trp-Ala-Lys)-β-Ala-(Arg)₅-Gln-(Arg)₃-NH₂;
Ac-Nle-c(Asp-His-D-Phe-Arg-Trp-Lys)-β-Ala-Tyr-Gly-(Arg)₅-Gln-(Arg)₃-NH₂;
Ac-Nle-c(Asp-His-D-Phe-Arg-Trp-Lys)-β-Ala-Gly-(Arg)₅-Gln-(Arg)₃-NH₂;
Ac-Nle-c(Asp-His-D-Phe-Arg-Trp-Lys)-β-Ala-(Arg)₅-Gln-(Arg)₃-NH₂;
Ac-Nle-c(Asp-His-D-Phe-Arg-Trp-Lys)-(β-Ala)₂-Tyr-Gly-(Arg)₅-Gln-(Arg)₃-NH₂;
Ac-Nle-c(Asp-His-D-Phe-Arg-Trp-Lys)-(β-Ala)₂-Gly-(Arg)₅-Gln-(Arg)₃-NH₂;
Ac-Nle-c(Asp-His-D-Phe-Arg-Trp-Lys)-(β-Ala)₂-(Arg)₅-Gln-(Arg)₃-NH₂;
Ac-Nle-c(Asp-His-D-Phe-Arg-Trp-Lys)-Doc-Tyr-Gly-(Arg)₅-Gln-(Arg)₃-NH₂;
Ac-Nle-c(Asp-His-D-Phe-Arg-Trp-Lys)-Doc-Gly-(Arg)₅-Gln-(Arg)₃-NH₂;
Ac-Nle-c(Asp-His-D-Phe-Arg-Trp-Lys)-Doc-(Arg)₅-Gln-(Arg)₃-NH₂;
Ac-Nle-c(Asp-His-D-Phe-Arg-Trp-Lys)-(Doc)₂-Tyr-Gly-(Arg)₅-Gln-(Arg)₃-NH₂;
Ac-Nle-c(Asp-His-D-Phe-Arg-Trp-Lys)-(Doc)₂-Gly-(Arg)₅-Gln-(Arg)₃-NH₂;
Ac-Nle-c(Asp-His-D-Phe-Arg-Trp-Lys)-(Doc)₂-(Arg)₅-Gln-(Arg)₃-NH₂;
Ac-Nle-c(Asp-His-D-Phe-Arg-Trp-Lys)-β-Ala-Tyr-Gly-(Arg)₅-Gln-(Arg)₄-NH₂;
Ac-Nle-c(Asp-His-D-Phe-Arg-Trp-Lys)-β-Ala-Gly-(Arg)₅-Gln-(Arg)₄-NH₂;
Ac-Nle-c(Asp-His-D-Phe-Arg-Trp-Lys)-β-Ala-(Arg)₅-Gln-(Arg)₄-NH₂;
Ac-Nle-c(Asp-His-D-Phe-Arg-Trp-Lys)-(β-Ala)₂-Tyr-Gly-(Arg)₅-Gln-(Arg)₄-NH₂;
Ac-Nle-c(Asp-His-D-Phe-Arg-Trp-Lys)-(β-Ala)₂-Gly-(Arg)₅-Gln-(Arg)₄-NH₂;
Ac-Nle-c(Asp-His-D-Phe-Arg-Trp-Lys)-(β-Ala)₂-(Arg)₅-Gln-(Arg)₄-NH₂;
Ac-Nle-c(Asp-His-D-Phe-Arg-Trp-Lys)-Doc-Tyr-Gly-(Arg)₅-Gln-(Arg)₄-NH₂;
Ac-Nle-c(Asp-His-D-Phe-Arg-Trp-Lys)-Doc-Gly-(Arg)₅-Gln-(Arg)₄-NH₂;
Ac-Nle-c(Asp-His-D-Phe-Arg-Trp-Lys)-Doc-(Arg)₅-Gln-(Arg)₄-NH₂;
Ac-Nle-c(Asp-His-D-Phe-Arg-Trp-Lys)-(Doc)₂-Tyr-Gly-(Arg)₅-Gln-(Arg)₄-NH₂;
Ac-Nle-c(Asp-His-D-Phe-Arg-Trp-Lys)-(Doc)₂-Gly-(Arg)₅-Gln-(Arg)₄-NH₂;
Ac-Nle-c(Asp-His-D-Phe-Arg-Trp-Lys)-(Doc)₂-(Arg)₅-Gln-(Arg)₄-NH₂;
Ac-Nle-c(Asp-His-D-Phe-Arg-Trp-β-Ala-Lys)-β-Ala-Tyr-Gly-(Arg)₅-Gln-(Arg)₃-NH₂;
Ac-Nle-c(Asp-His-D-Phe-Arg-Trp-β-Ala-Lys)-β-Ala-(Arg)₅-Gln-(Arg)₃-NH₂;
Ac-Nle-c(Cys-His-D-Phe-Arg-Trp-Ahx-Cys)-β-Ala-Tyr-Gly-(Arg)₅-Gln-(Arg)₃-NH₂;
Ac-Nle-c(Cys-His-D-Phe-Arg-Trp-Ahx-Cys)-β-Ala-(Arg)₅-Gln-(Arg)₃-NH₂;
D-Phe-c(Cys-His-D-Phe-Arg-Trp-β-Ala-D-Cys)-Thr-β-Ala-Tyr-Gly-(Arg)₅-Gln-(Arg)₃-NH₂;
D-Phe-c(Cys-His-D-Phe-Arg-Trp-β-Ala-D-Cys)-Thr-β-Ala-(Arg)₅-Gln-(Arg)₃-NH₂;
Ac-Nle-c(Cys-His-D-Phe-Arg-Trp-Apn-Cys)-β-Ala-Tyr-Gly-(Arg)₅-Gln-(Arg)₃-NH₂;
Ac-Nle-c(Cys-His-D-Phe-Arg-Trp-Apn-Cys)-β-Ala-(Arg)₅-Gln-(Arg)₃-NH₂;
Ac-Cha-c(Asp-His-D-Phe-Arg-Trp-Gaba-Lys)-β-Ala-Tyr-Gly-(Arg)₅-Gln-(Arg)₃-NH₂;
Ac-Cha-c(Asp-His-D-Phe-Arg-Trp-Gaba-Lys)-β-Ala-(Arg)₅-Gln-(Arg)₃-NH₂;
Ac-Nle-c(Asp-His-D-Phe-Arg-Trp-Gaba-Lys)-β-Ala-Tyr-Gly-(Arg)₅-Gln-(Arg)₃-NH₂;
Ac-Nle-c(Asp-His-D-Phe-Arg-Trp-Gaba-Lys)-β-Ala-(Arg)₅-Gln-(Arg)₃-NH₂;
Ac-Chg-c(Asp-His-D-Phe-Arg-Trp-Gaba-Lys)-β-Ala-Tyr-Gly-(Arg)₅-Gln-(Arg)₃-NH₂;
Ac-Chg-c(Asp-His-D-Phe-Arg-Trp-Gaba-Lys)-β-Ala-(Arg)₅-Gln-(Arg)₃-NH₂;
Ac-hCha-c(Asp-His-D-Phe-Arg-Trp-Gaba-Lys)-β-Ala-Tyr-Gly-(Arg)₅-Gln-(Arg)₃-NH₂;
Ac-hCha-c(Asp-His-D-Phe-Arg-Trp-Gaba-Lys)-β-Ala-(Arg)₅-Gln-(Arg)₃-NH₂;
Ac-hCha-c(Asp-His-D-Phe-Arg-Trp-Gaba-Lys)-(β-Ala)₂-Tyr-Gly-(Arg)₅-Gln-(Arg)₃-NH₂;
Ac-hCha-c(Asp-His-D-Phe-Arg-Trp-Gaba-Lys)-(β-Ala)₂-(Arg)₅-Gln-(Arg)₃-NH₂;
Ac-hCha-c(Asp-His-D-Phe-Arg-Trp-Gaba-Lys)-Doc-Tyr-Gly-(Arg)₅-Gln-(Arg)₃-NH₂;
Ac-hCha-c(Asp-His-D-Phe-Arg-Trp-Gaba-Lys)-Doc-(Arg)₅-Gln-(Arg)₃-NH₂;
Ac-hCha-c(Asp-His-D-Phe-Arg-Trp-Gaba-Lys)-(Doc)₂-Tyr-Gly-(Arg)₅-Gln-(Arg)₃-NH₂;
Ac-hCha-c(Asp-His-D-Phe-Arg-Trp-Gaba-Lys)-(Doc)₂-(Arg)₅-Gln-(Arg)₃-NH₂;
Ac-hCha-c(Asp-His-D-Phe-Arg-Trp-Gaba-Lys)-β-Ala-Tyr-Gly-(Arg)₅-Gln-(Arg)₄-NH₂;
Ac-hCha-c(Asp-His-D-Phe-Arg-Trp-Gaba-Lys)-β-Ala-(Arg)₅-Gln-(Arg)₄-NH₂;
Ac-hCha-c(Asp-His-D-Phe-Arg-Trp-Gaba-Lys)-(β-Ala)₂-Tyr-Gly-(Arg)₅-Gln-(Arg)₄-NH₂;
Ac-hCha-c(Asp-His-D-Phe-Arg-Trp-Gaba-Lys)-(β-Ala)₂-(Arg)₅-Gln-(Arg)₄-NH₂;
Ac-hCha-c(Asp-His-D-Phe-Arg-Trp-Gaba-Lys)-Doc-Tyr-Gly-(Arg)₅-Gln-(Arg)₄-NH₂;
Ac-hCha-c(Asp-His-D-Phe-Arg-Trp-Gaba-Lys)-Doc-(Arg)₅-Gln-(Arg)₄-NH₂;
Ac-hCha-c(Asp-His-D-Phe-Arg-Trp-Gaba-Lys)-(Doc)₂-Tyr-Gly-(Arg)₅-Gln-(Arg)₄-NH₂;
Ac-hCha-c(Asp-His-D-Phe-Arg-Trp-Gaba-Lys)-(Doc)₂-(Arg)₅-Gln-(Arg)₄-NH₂;
Ac-D-Chg-c(Asp-His-D-Phe-Arg-Trp-Gaba-Lys)-β-Ala-Tyr-Gly-(Arg)₅-Gln-(Arg)₃-NH₂;
Ac-D-Chg-c(Asp-His-D-Phe-Arg-Trp-Gaba-Lys)-β-Ala-(Arg)₅-Gln-(Arg)₃-NH₂;
Ac-hPhe-c(Asp-His-D-Phe-Arg-Trp-Gaba-Lys)-β-Ala-Tyr-Gly-(Arg)₅-Gln-(Arg)₃-NH₂;
Ac-hPhe-c(Asp-His-D-Phe-Arg-Trp-Gaba-Lys)-β-Ala-(Arg)₅-Gln-(Arg)₃-NH₂;
Ac-Nle-c(Cys-His-D-Phe-Arg-D-Trp-Apn-Cys)-β-Ala-Tyr-Gly-(Arg)₅-Gln-(Arg)₃-NH₂;
Ac-Nle-c(Cys-His-D-Phe-Arg-D-Trp-Apn-Cys)-β-Ala-(Arg)₅-Gln-(Arg)₃-NH₂;
Ac-Nle-c(Cys-His-D-Phe-Arg-D-Trp-Ahx-Cys)-β-Ala-Tyr-Gly-(Arg)₅-Gln-(Arg)₃-NH₂;
Ac-Nle-c(Cys-His-D-Phe-Arg-D-Trp-Ahx-Cys)-β-Ala-(Arg)₅-Gln-(Arg)₃-NH₂;
Ac-Nle-c(Cys-His-D-Phe-Arg-D-Trp-β-Ala-Cys)-β-Ala-Tyr-Gly-(Arg)₅-Gln-(Arg)₃-NH₂;
Ac-Nle-c(Cys-His-D-Phe-Arg-D-Trp-β-Ala-Cys)-β-Ala-(Arg)₅-Gln-(Arg)₃-NH₂;
Ac-Nle-c(Cys-D-Ala-His-D-Phe-Arg-Trp-Pen)-β-Ala-Tyr-Gly-(Arg)₅-Gln-(Arg)₃-NH₂;
Ac-Nle-c(Cys-D-Ala-His-D-Phe-Arg-Trp-Pen)-β-Ala-Gly-(Arg)₅-Gln-(Arg)₃-NH₂;
Ac-Nle-c(Cys-D-Ala-His-D-Phe-Arg-Trp-Pen)-β-Ala-(Arg)₅-Gln-(Arg)₃-NH₂;
Ac-Nle-c(Cys-D-Ala-His-D-Phe-Arg-Trp-Pen)-(β-Ala)₂-Tyr-Gly-(Arg)₅-Gln-(Arg)₃-NH₂;
Ac-Nle-c(Cys-D-Ala-His-D-Phe-Arg-Trp-Pen)-(β-Ala)₂-Gly-(Arg)₅-Gln-(Arg)₃-NH₂;
Ac-Nle-c(Cys-D-Ala-His-D-Phe-Arg-Trp-Pen)-(β-Ala)₂-(Arg)₅-Gln-(Arg)₃-NH₂;
Ac-Nle-c(Cys-D-Ala-His-D-Phe-Arg-Trp-Pen)-Doc-Tyr-Gly-(Arg)₅-Gln-(Arg)₃-NH₂;
Ac-Nle-c(Cys-D-Ala-His-D-Phe-Arg-Trp-Pen)-Doc-Gly-(Arg)₅-Gln-(Arg)₃-NH₂;
Ac-Nle-c(Cys-D-Ala-His-D-Phe-Arg-Trp-Pen)-Doc-(Arg)₅-Gln-(Arg)₃-NH₂;
Ac-Nle-c(Cys-D-Ala-His-D-Phe-Arg-Trp-Pen)-(Doc)₂-Tyr-Gly-(Arg)₅-Gln-(Arg)₃-NH₂;
Ac-Nle-c(Cys-D-Ala-His-D-Phe-Arg-Trp-Pen)-(Doc)₂-Gly-(Arg)₅-Gln-(Arg)₃-NH₂;
Ac-Nle-c(Cys-D-Ala-His-D-Phe-Arg-Trp-Pen)-(Doc)₂-(Arg)₅-Gln-(Arg)₃-NH₂;
D-Phe-c(Cys-His-D-(Et)Tyr-Arg-Trp-β-Ala-D-Cys)-β-Ala-Tyr-Gly-(Arg)₅-Gln-(Arg)₃-NH₂;
D-Phe-c(Cys-His-D-(Et)Tyr-Arg-Trp-β-Ala-D-Cys)-β-Ala-(Arg)₅-Gln-(Arg)₃-NH₂;
D-Phe-c(Cys-His-D-(Et)Tyr-Arg-Trp-β-Ala-D-Cys)-β-Ala-Gly-(Arg)₅-Gln-(Arg)₃-NH₂;
D-Phe-c(Cys-His-D-(Et)Tyr-Arg-Trp-β-Ala-D-Cys)-β-Ala-(Arg)₅-Gln-(Arg)₄-NH₂;
D-Phe-c(Cys-His-D-(Et)Tyr-Arg-Trp-β-Ala-D-Cys)-(β-Ala)₂-Tyr-Gly-(Arg)₅-Gln-(Arg)₃-NH₂;
D-Phe-c(Cys-His-D-(Et)Tyr-Arg-Trp-β-Ala-D-Cys)-(β-Ala)₂-(Arg)₅-Gln-(Arg)₃-NH₂;
D-Phe-c(Cys-His-D-(Et)Tyr-Arg-Trp-β-Ala-D-Cys)-(β-Ala)₂-Gly-(Arg)₅-Gln-(Arg)₃-NH₂;
D-Phe-c(Cys-His-D-(Et)Tyr-Arg-Trp-β-Ala-D-Cys)-(β-Ala)₂-(Arg)₅-Gln-(Arg)₄-NH₂;
D-Phe-c(Cys-His-D-(Et)Tyr-Arg-Trp-β-Ala-D-Cys)-Doc-Tyr-Gly-(Arg)₅-Gln-(Arg)₃-NH₂;
D-Phe-c(Cys-His-D-(Et)Tyr-Arg-Trp-β-Ala-D-Cys)-Doc-(Arg)₅-Gln-(Arg)₃-NH₂;
D-Phe-c(Cys-His-D-(Et)Tyr-Arg-Trp-β-Ala-D-Cys)-Doc-Gly-(Arg)₅-Gln-(Arg)₃-NH₂;
D-Phe-c(Cys-His-D-(Et)Tyr-Arg-Trp-β-Ala-D-Cys)-Doc-(Arg)₅-Gln-(Arg)₄-NH₂;
D-Phe-c(Cys-His-D-(Et)Tyr-Arg-Trp-β-Ala-D-Cys)-(Doc)₂-Tyr-Gly-(Arg)₅-Gln-(Arg)₃-NH₂;
D-Phe-c(Cys-His-D-(Et)Tyr-Arg-Trp-β-Ala-D-Cys)-(Doc)₂-(Arg)₅-Gln-(Arg)₃-NH₂;
D-Phe-c(Cys-His-D-(Et)Tyr-Arg-Trp-β-Ala-D-Cys)-(Doc)₂-Gly-(Arg)₅-Gln-(Arg)₃-NH₂;
D-Phe-c(Cys-His-D-(Et)Tyr-Arg-Trp-β-Ala-D-Cys)-(Doc)₂-(Arg)₅-Gln-(Arg)₄-NH₂;
D-Phe-c(Cys-His-D-(Et)Tyr-hArg-Trp-β-Ala-D-Cys)-Thr-β-Ala-Tyr-Gly-(Arg)₅-Gln-(Arg)₃-NH₂;
D-Phe-c(Cys-His-D-(Et)Tyr-hArg-Trp-β-Ala-D-Cys)-Thr-β-Ala-(Arg)₅-Gln-(Arg)₃-NH₂;
D-Phe-c(Cys-His-D-(Et)Tyr-hArg-Trp-β-Ala-D-Cys)-Thr-(β-Ala)₂-Tyr-Gly-(Arg)₅-Gln-(Arg)₃-NH₂;
D-Phe-c(Cys-His-D-(Et)Tyr-hArg-Trp-β-Ala-D-Cys)-Thr-(β-Ala)₂-(Arg)₅-Gln-(Arg)₃-NH₂;
D-Phe-c(Cys-His-D-(Et)Tyr-hArg-Trp-β-Ala-D-Cys)-Thr-Doc-Tyr-Gly-(Arg)₅-Gln-(Arg)₃-NH₂;
D-Phe-c(Cys-His-D-(Et)Tyr-hArg-Trp-β-Ala-D-Cys)-Thr-Doc-(Arg)₅-Gln-(Arg)₃-NH₂;
D-Phe-c(Cys-His-D-(Et)Tyr-hArg-Trp-β-Ala-D-Cys)-Thr-(Doc)₂-Tyr-Gly-(Arg)₅-Gln-(Arg)₃-NH₂;
D-Phe-c(Cys-His-D-(Et)Tyr-hArg-Trp-β-Ala-D-Cys)-Thr-β-Ala-Tyr-Gly-(Arg)₅-Gln-(Arg)₄-NH₂;
D-Phe-c(Cys-His-D-(Et)Tyr-hArg-Trp-β-Ala-D-Cys)-Thr-β-Ala-(Arg)₅-Gln-(Arg)₄-NH₂;
D-Phe-c(Cys-His-D-(Et)Tyr-hArg-Trp-β-Ala-D-Cys)-Thr-(β-Ala)₂-Tyr-Gly-(Arg)₅-Gln-(Arg)₄-NH₂;
D-Phe-c(Cys-His-D-(Et)Tyr-hArg-Trp-β-Ala-D-Cys)-Thr-(β-Ala)₂-(Arg)₅-Gln-(Arg)₄-NH₂;
D-Phe-c(Cys-His-D-(Et)Tyr-hArg-Trp-β-Ala-D-Cys)-Thr-Doc-Tyr-Gly-(Arg)₅-Gln-(Arg)₄-NH₂;
D-Phe-c(Cys-His-D-(Et)Tyr-hArg-Trp-β-Ala-D-Cys)-Thr-Doc-(Arg)₅-Gln-(Arg)₄-NH₂;
D-Phe-c(Cys-His-D-(Et)Tyr-hArg-Trp-β-Ala-D-Cys)-Thr-(Doc)₂-Tyr-Gly-(Arg)₅-Gln-(Arg)₄-NH₂;
D-Phe-c(Cys-His-D-(Et)Tyr-hArg-Trp-β-Ala-D-Cys)-Thr-(Doc)₂-(Arg)₅-Gln-(Arg)₄-NH₂;
D-Phe-c(Cys-His-D-(Et)Tyr-hArg-Bip-β-Ala-D-Cys)-Thr-β-Ala-Tyr-Gly-(Arg)₅-Gln-(Arg)₃-NH₂;
D-Phe-c(Cys-His-D-(Et)Tyr-hArg-Bip-β-Ala-D-Cys)-Thr-β-Ala-Tyr-Gly-(Arg)₅-Gln-(Arg)₄-NH₂;
D-Phe-c(Cys-His-D-(Et)Tyr-hArg-Bip-β-Ala-D-Cys)-Thr-β-Ala-(Arg)₅-Gln-(Arg)₃-NH₂;
D-Phe-c(Cys-His-D-(Et)Tyr-hArg-Bip-β-Ala-D-Cys)-Thr-(β-Ala)₂-Tyr-Gly-(Arg)₅-Gln-(Arg)₃-NH₂;
D-Phe-c(Cys-His-D-(Et)Tyr-hArg-Bip-β-Ala-D-Cys)-Thr-(β-Ala)₂-(Arg)₅-Gln-(Arg)₃-NH₂;
D-Phe-c(Cys-His-D-(Et)Tyr-hArg-Bip-β-Ala-D-Cys)-Thr-Doc-Tyr-Gly-(Arg)₅-Gln-(Arg)₃-NH₂;
D-Phe-c(Cys-His-D-(Et)Tyr-hArg-Bip-β-Ala-D-Cys)-Thr-Doc-Tyr-Gly-(Arg)₅-Gln-(Arg)₄-NH₂;
D-Phe-c(Cys-His-D-(Et)Tyr-hArg-Bip-β-Ala-D-Cys)-Thr-Doc-(Arg)₅-Gln-(Arg)₃-NH₂;
D-Phe-c(Cys-His-D-(Et)Tyr-hArg-Bip-β-Ala-D-Cys)-Thr-(Doc)₂-Tyr-Gly-(Arg)₅-Gln-(Arg)₃-NH₂;
D-Phe-c(Cys-His-D-(Et)Tyr-hArg-Bip-β-Ala-D-Cys)-Thr-(Doc)₂-(Arg)₅-Gln-(Arg)₃-NH₂;
Ac-Nle-c(Cys-D-Ala-His-D-Phe-Arg-Trp-Gly-Cys)-β-Ala-Tyr-Gly-(Arg)₅-Gln-(Arg)₃-NH₂;
Ac-Nle-c(Cys-D-Ala-His-D-Phe-Arg-Trp-Gly-Cys)-β-Ala-(Arg)₅-Gln-(Arg)₃-NH₂;
Nle-c(Cys-His-D-Phe-Arg-Trp-Apn-Cys)-β-Ala-Tyr-Gly-(Arg)₅-Gln-(Arg)₃-NH₂;
Nle-c(Cys-His-D-Phe-Arg-Trp-Apn-Cys)-β-Ala-(Arg)₅-Gln-(Arg)₃-NH₂;
Nle-c(Cys-His-D-Phe-Arg-Trp-Apn-Cys)-(β-Ala)₂-Tyr-Gly-(Arg)₅-Gln-(Arg)₃-NH₂;
Nle-c(Cys-His-D-Phe-Arg-Trp-Apn-Cys)-(β-Ala)₂-(Arg)₅-Gln-(Arg)₃-NH₂;
Nle-c(Cys-His-D-Phe-Arg-Trp-Apn-Cys)-β-Ala-Tyr-Gly-(Arg)₅-Gln-(Arg)₄-NH₂;
Nle-c(Cys-His-D-Phe-Arg-Trp-Apn-Cys)-β-Ala-(Arg)₅-Gln-(Arg)₄-NH₂;
Nle-c(Cys-His-D-Phe-Arg-Trp-Apn-Cys)-(β-Ala)₂-Tyr-Gly-(Arg)₅-Gln-(Arg)₄-NH₂;
Nle-c(Cys-His-D-Phe-Arg-Trp-Apn-Cys)-(β-Ala)₂-(Arg)₅-Gln-(Arg)₄-NH₂;
Nle-c(Cys-His-D-Phe-Arg-Trp-Apn-Cys)-Doc-Tyr-Gly-(Arg)₅-Gln-(Arg)₃-NH₂;
Nle-c(Cys-His-D-Phe-Arg-Trp-Apn-Cys)-Doc-(Arg)₅-Gln-(Arg)₃-NH₂;
Nle-c(Cys-His-D-Phe-Arg-Trp-Apn-Cys)-(Doc)₂-Tyr-Gly-(Arg)₅-Gln-(Arg)₃-NH₂;
Nle-c(Cys-His-D-Phe-Arg-Trp-Apn-Cys)-(Doc)₂-(Arg)₅-Gln-(Arg)₃-NH₂;
Nle-c(Cys-His-D-Phe-Arg-Trp-Apn-Cys)-Doc-Tyr-Gly-(Arg)₅-Gln-(Arg)₄-NH₂;
Nle-c(Cys-His-D-Phe-Arg-Trp-Apn-Cys)-Doc-(Arg)₅-Gln-(Arg)₄-NH₂;
Nle-c(Cys-His-D-Phe-Arg-Trp-Apn-Cys)-(Doc)₂-Tyr-Gly-(Arg)₅-Gln-(Arg)₄-NH₂;
Nle-c(Cys-His-D-Phe-Arg-Trp-Apn-Cys)-(Doc)₂-(Arg)₅-Gln-(Arg)₄-NH₂;
Ac-Nle-c(Cys-D-Leu-His-D-Phe-Arg-Trp-Cys)-β-Ala-Tyr-Gly-(Arg)₅-Gln-(Arg)₃-NH₂;
Ac-Nle-c(Cys-D-Leu-His-D-Phe-Arg-Trp-Cys)-β-Ala-(Arg)₅-Gln-(Arg)₃-NH₂;
Ac-Nle-c(Cys-D-Leu-His-D-Phe-Arg-Trp-Cys)-(β-Ala)₂-Tyr-Gly-(Arg)₅-Gln-(Arg)₃-NH₂;
Ac-Nle-c(Cys-D-Leu-His-D-Phe-Arg-Trp-Cys)-(β-Ala)₂-(Arg)₅-Gln-(Arg)₃-NH₂;
Ac-Nle-c(Cys-D-Leu-His-D-Phe-Arg-Trp-Cys)-Doc-Tyr-Gly-(Arg)₅-Gln-(Arg)₃-NH₂;
Ac-Nle-c(Cys-D-Leu-His-D-Phe-Arg-Trp-Cys)-Doc-(Arg)₅-Gln-(Arg)₃-NH₂;
Ac-Nle-c(Cys-D-Leu-His-D-Phe-Arg-Trp-Cys)-(Doc)₂-Tyr-Gly-(Arg)₅-Gln-(Arg)₃-NH₂;
Ac-Nle-c(Cys-D-Leu-His-D-Phe-Arg-Trp-Cys)-(Doc)₂-(Arg)₅-Gln-(Arg)₃-NH₂;
Ac-Nle-c(Cys-D-Leu-His-D-Phe-Arg-Trp-Cys)-β-Ala-Tyr-Gly-(Arg)₅-Gln-(Arg)₄-NH₂;
Ac-Nle-c(Cys-D-Leu-His-D-Phe-Arg-Trp-Cys)-β-Ala-(Arg)₅-Gln-(Arg)₄-NH₂;
Ac-Nle-c(Cys-D-Leu-His-D-Phe-Arg-Trp-Cys)-(β-Ala)₂-Tyr-Gly-(Arg)₅-Gln-(Arg)₄-NH₂;
Ac-Nle-c(Cys-D-Leu-His-D-Phe-Arg-Trp-Cys)-(β-Ala)₂-(Arg)₅-Gln-(Arg)₄-NH₂;
Ac-Nle-c(Cys-D-Leu-His-D-Phe-Arg-Trp-Cys)-Doc-Tyr-Gly-(Arg)₅-Gln-(Arg)₄-NH₂;
Ac-Nle-c(Cys-D-Leu-His-D-Phe-Arg-Trp-Cys)-Doc-(Arg)₅-Gln-(Arg)₄-NH₂;
Ac-Nle-c(Cys-D-Leu-His-D-Phe-Arg-Trp-Cys)-(Doc)₂-Tyr-Gly-(Arg)₅-Gln-(Arg)₄-NH₂;
Ac-Nle-c(Cys-D-Leu-His-D-Phe-Arg-Trp-Cys)-(Doc)₂-(Arg)₅-Gln-(Arg)₄-NH₂;
Ac-Nle-c(Cys-D-Cha-His-D-Phe-Arg-Trp-Cys)-β-Ala-Tyr-Gly-(Arg)₅-Gln-(Arg)₃-NH₂;
Ac-Nle-c(Cys-D-Cha-His-D-Phe-Arg-Trp-Cys)-β-Ala-(Arg)₅-Gln-(Arg)₃-NH₂;
Ac-Nle-c(Cys-D-Cha-His-D-Phe-Arg-Trp-Cys)-(β-Ala)₂-Tyr-Gly-(Arg)₅-Gln-(Arg)₃-NH₂;
Ac-Nle-c(Cys-D-Cha-His-D-Phe-Arg-Trp-Cys)-(β-Ala)₂-(Arg)₅-Gln-(Arg)₃-NH₂;
Ac-Nle-c(Cys-D-Cha-His-D-Phe-Arg-Trp-Cys)-Doc-Tyr-Gly-(Arg)₅-Gln-(Arg)₃-NH₂;
Ac-Nle-c(Cys-D-Cha-His-D-Phe-Arg-Trp-Cys)-Doc-(Arg)₅-Gln-(Arg)₃-NH₂;
Ac-Nle-c(Cys-D-Cha-His-D-Phe-Arg-Trp-Cys)-(Doc)₂-Tyr-Gly-(Arg)₅-Gln-(Arg)₃-NH₂;
Ac-Nle-c(Cys-D-Cha-His-D-Phe-Arg-Trp-Cys)-(Doc)₂-(Arg)₅-Gln-(Arg)₃-NH₂;
Ac-Nle-c(Cys-D-Cha-His-D-Phe-Arg-Trp-Cys)-β-Ala-Tyr-Gly-(Arg)₅-Gln-(Arg)₄-NH₂;
Ac-Nle-c(Cys-D-Cha-His-D-Phe-Arg-Trp-Cys)-β-Ala-(Arg)₅-Gln-(Arg)₄-NH₂;
Ac-Nle-c(Cys-D-Cha-His-D-Phe-Arg-Trp-Cys)-(β-Ala)₂-Tyr-Gly-(Arg)₅-Gln-(Arg)₄-NH₂;
Ac-Nle-c(Cys-D-Cha-His-D-Phe-Arg-Trp-Cys)-(β-Ala)₂-(Arg)₅-Gln-(Arg)₄-NH₂;
Ac-Nle-c(Cys-D-Cha-His-D-Phe-Arg-Trp-Cys)-Doc-Tyr-Gly-(Arg)₅-Gln-(Arg)₄-NH₂;
Ac-Nle-c(Cys-D-Cha-His-D-Phe-Arg-Trp-Cys)-Doc-(Arg)₅-Gln-(Arg)₄-NH₂;
Ac-Nle-c(Cys-D-Cha-His-D-Phe-Arg-Trp-Cys)-(Doc)₂-Tyr-Gly-(Arg)₅-Gln-(Arg)₄-NH₂;
Ac-Nle-c(Cys-D-Cha-His-D-Phe-Arg-Trp-Cys)-(Doc)₂-(Arg)₅-Gln-(Arg)₄-NH₂;
Nle-c(Cys-His-D-Phe-Arg-Trp-Gaba-Cys)-β-Ala-Tyr-Gly-(Arg)₅-Gln-(Arg)₃-NH₂;
Nle-c(Cys-His-D-Phe-Arg-Trp-Gaba-Cys)-β-Ala-(Arg)₅-Gln-(Arg)₃-NH₂;
Nle-c(Cys-His-D-Phe-Arg-Trp-Gaba-Cys)-(β-Ala)₂-Tyr-Gly-(Arg)₅-Gln-(Arg)₃-NH₂;
Nle-c(Cys-His-D-Phe-Arg-Trp-Gaba-Cys)-(β-Ala)₂-(Arg)₅-Gln-(Arg)₃-NH₂;
Nle-c(Cys-His-D-Phe-Arg-Trp-Gaba-Cys)-β-Ala-Tyr-Gly-(Arg)₅-Gln-(Arg)₄-NH₂;
Nle-c(Cys-His-D-Phe-Arg-Trp-Gab a-Cys)-β-Ala-(Arg)₅-Gln-(Arg)₄-NH₂;
Nle-c(Cys-His-D-Phe-Arg-Trp-Gaba-Cys)-(β-Ala)₂-Tyr-Gly-(Arg)₅-Gln-(Arg)₄-NH₂;
Nle-c(Cys-His-D-Phe-Arg-Trp-Gaba-Cys)-(β-Ala)₂-(Arg)₅-Gln-(Arg)₄-NH₂;
Nle-c(Cys-His-D-Phe-Arg-Trp-Gaba-Cys)-Doc-Tyr-Gly-(Arg)₅-Gln-(Arg)₃-NH₂;
Nle-c(Cys-His-D-Phe-Arg-Trp-Gaba-Cys)-Doc-(Arg)₅-Gln-(Arg)₃-NH₂;
Nle-c(Cys-His-D-Phe-Arg-Trp-Gaba-Cys)-(Doc)₂-Tyr-Gly-(Arg)₅-Gln-(Arg)₃-NH₂;
Nle-c(Cys-His-D-Phe-Arg-Trp-Gaba-Cys)-(Doc)₂-(Arg)₅-Gln-(Arg)₃-NH₂;
Nle-c(Cys-His-D-Phe-Arg-Trp-Gaba-Cys)-Doc-Tyr-Gly-(Arg)₅-Gln-(Arg)₄-NH₂;
Nle-c(Cys-His-D-Phe-Arg-Trp-Gaba-Cys)-Doc-(Arg)₅-Gln-(Arg)₄-NH₂;
Nle-c(Cys-His-D-Phe-Arg-Trp-Gaba-Cys)-(Doc)₂-Tyr-Gly-(Arg)₅-Gln-(Arg)₄-NH₂;
Nle-c(Cys-His-D-Phe-Arg-Trp-Gaba-Cys)-(Doc)₂-(Arg)₅-Gln-(Arg)₄-NH₂;
Ac-c(Cys-Glu-His-D-4-Br-Phe-Arg-Trp-Gly-Cys)-(Pro)₂-Lys-Asp-NH₂;
Ac-c(Cys-Glu-His-D-2-Nal-Arg-Trp-Ala-Cys)-(Pro)₂-Lys-Asp-NH₂;
Ac-c(Cys-Glu-His-D-2-Nal-Arg-2-Nal-Ala-Cys)-(Pro)₂-Lys-Asp-NH₂;
Ac-c(Cys-Glu-His-D-2-Nal-Arg-1-Nal-Ala-Cys)-(Pro)₂-Lys-Asp-NH₂;
Ac-c(Cys-Glu-His-D-2-Nal-Arg-Bal-Ala-Cys)-(Pro)₂-Lys-Asp-NH₂;
Ac-c(Cys-Glu-His-D-2-Nal-Arg-2-Nal-β-Ala-Cys)-(Pro)₂-Lys-Asp-NH₂;
Ac-c(Cys-Glu-His-D-2-Nal-Arg-2-Nal-Aib-Cys)-(Pro)₂-Lys-Asp-NH₂;
c[Hydantoin(C(O)-(Cys-D-Ala))-His-D-Phe-Arg-Trp-Cys]-NH₂;
c[Hydantoin(C(O)-(hCys-D-Ala))-His-D-Phe-Arg-Trp-Cys]-NH₂;
c[Hydantoin(C(O)-(Cys-D-Ala))-His-D-2-Nal-Arg-Trp-Cys]-NH₂;
c[Hydantoin(C(O)-(hCys-D-Ala))-His-D-2-Nal-Arg-Trp-Cys]-NH₂;
c[Hydantoin(C(O)-(Asp-D-Ala))-His-D-Phe-Arg-Trp-Lys]-NH₂;
c[Hydantoin(C(O)-(Asp-D-Ala))-His-D-Phe-Arg-Trp-Orn]-NH₂;
c[Hydantoin(C(O)-(Asp-D-Ala))-His-D-Phe-Arg-Trp-Dab]-NH₂;
c[Hydantoin(C(O)-(Asp-D-Ala))-His-D-Phe-Arg-Trp-Dap]-NH₂;
c[Hydantoin(C(O)-(Asp-His))-D-2-Nal-Arg-Trp-Lys]-NH₂;
c[Hydantoin(C(O)-(Asp-His))-D-Phe-Arg-Trp-Lys]-NH₂;
c[Hydantoin(C(O)-(Asp-A3c))-D-Phe-Arg-Trp-Lys]-NH₂;
c[Hydantoin(C(O)-(Asp-A5c))-D-Phe-Arg-Trp-Lys]-NH₂;
c[Hydantoin(C(O)-(Asp-A6c))-D-Phe-Arg-Trp-Lys]-NH₂;
c[Hydantoin(C(O)-(Asp-A3c))-D-2-Nal-Arg-Trp-Lys]-NH₂;
c[Hydantoin(C(O)-(Asp-A5c))-D-2-Nal-Arg-Trp-Lys]-NH₂;
c[Hydantoin(C(O)-(Asp-A6c))-D-2-Nal-Arg-Trp-Lys]-NH₂;
c[Hydantoin (C(O)-(Asp-A5c))-D-2-Nal-Arg-Trp-Lys]-NH₂;
c[Hydantoin(C(O)-(Asp-Aic))-D-Phe-Arg-Trp-Lys]-NH₂;
c[Hydantoin(C(O)-(Asp-Apc))-D-Phe-Arg-Trp-Lys]-NH₂;
c[Hydantoin(C(O)-(Asp-Aic))-D-2-Nal-Arg-Trp-Lys]-NH₂;
c[Hydantoin(C(O)-(Asp-Apc))-D-2-Nal-Arg-Trp-Lys]-NH₂;
c[Hydantoin-(C(O)-(Asp-Aic))-D-2-Nal-Arg-Trp-Lys]-NH₂;
c[Hydantoin-(C(O)-(Asp-Apc))-D-2-Nal-Arg-Trp-Lys]-NH₂;
c[Hydantoin(C(O)-(Glu-D-Ala))-His-D-Phe-Arg-Trp-Orn]-NH₂;
c[Hydantoin(C(O)-(Glu-D-Ala))-His-D-Phe-Arg-Trp-Dab]-NH₂;
c[Hydantoin(C(O)-(Glu-D-Ala))-His-D-Phe-Arg-Trp-Dap]-NH₂;
c[Hydantoin(C(O)-(Glu-His))-D-Phe-Arg-Trp-Dap]-NH₂;
Hydantoin(C(O)-(Arg-Gly))-c(Cys-Glu-His-D-Phe-Arg-Trp-Cys)-NH₂;
Hydantoin(C(O)-(Nle-Gly))-c(Cys-Glu-His-D-Phe-Arg-Trp-Cys)-NH₂;
Hydantoin(C(O)-(Gly-Gly))-c(Cys-Glu-His-D-Phe-Arg-Trp-Cys)-NH₂;
Hydantoin(C(O)-(Nle-Gly))-c(Cys-D-Ala-His-D-Phe-Arg-Trp-Cys)-NH₂;
Hydantoin(C(O)-(Gly-Gly))-c(Cys-D-Ala-His-D-Phe-Arg-Trp-Cys)-NH₂;
Hydantoin(C(O)-(Nle-Gly))-c(Cys-D-Ala-His-D-Phe-Arg-Trp-Pen)-NH₂;
Hydantoin(C(O)-(Gly-Gly))-c(Cys-D-Ala-His-D-Phe-Arg-Trp-Pen)-NH₂;
Hydantoin(C(O)-(Ala-Gly))-c(Cys-D-Ala-His-D-Phe-Arg-Trp-Cys)-NH₂;
Hydantoin(C(O)-(D-Ala-Gly))-c(Cys-D-Ala-His-D-Phe-Arg-Trp-Cys)-NH₂;
Hydantoin(C(O)-(Aib-Gly))-c(Cys-D-Ala-His-D-Phe-Arg-Trp-Cys)-NH₂;
Hydantoin(C(O)-(Val-Gly))-c(Cys-D-Ala-His-D-Phe-Arg-Trp-Cys)-NH₂;
Hydantoin(C(O)-(Ile-Gly))-c(Cys-D-Ala-His-D-Phe-Arg-Trp-Cys)-NH₂;
Hydantoin(C(O)-(Leu-Gly))-c(Cys-D-Ala-His-D-Phe-Arg-Trp-Cys)-NH₂;
Hydantoin(C(O)-(Gly-Gly))-c(Cys-Glu-His-D-2-Nal-Arg-Trp-Cys)-NH₂;
Hydantoin(C(O)-(Nle-Gly))-c(Cys-Glu-His-D-2-Nal-Arg-Trp-Cys)-NH₂;
Hydantoin(C(O)-(D-Arg-Gly))-c(Cys-Glu-His-D-Phe-Arg-Trp-Cys)-NH₂;
Hydantoin(C(O)-(D-Arg-Gly))-c(Cys-D-Ala-His-D-Phe-Arg-Trp-Cys)-NH₂;
Hydantoin(C(O)-(Arg-Gly))-c(Cys-D-Ala-His-D-Phe-Arg-Trp-Cys)-NH₂;
Hydantoin(C(O)-(D-Arg-Gly))-c(Cys-D-Ala-His-D-2-Nal-Arg-Trp-Cys)-NH₂;
Hydantoin(C(O)-(Arg-Gly))-c(Cys-D-Ala-His-D-2-Nal-Arg-Trp-Cys)-NH₂;
Hydantoin(C(O)-(Ala-Nle))-c(Cys-Glu-His-D-Phe-Arg-Trp-Cys)-NH₂;
Hydantoin(C(O)-(Val-Nle))-c(Cys-Glu-His-D-Phe-Arg-Trp-Cys)-NH₂;
Hydantoin(C(O)-(Gly-Nle))-c(Cys-Glu-His-D-Phe-Arg-Trp-Cys)-NH₂;
Hydantoin(C(O)-(A6c-Nle))-c(Cys-D-Ala-His-D-Phe-Arg-Trp-Cys)-NH₂;
Hydantoin(C(O)-(Gly-Nle))-c(Cys-D-Ala-His-D-Phe-Arg-Trp-Cys)-NH₂;
Hydantoin(C(O)-(Ala-Nle))-c(Cys-D-Ala-His-D-Phe-Arg-Trp-Cys)-NH₂;
Hydantoin(C(O)-(D-Ala-Nle))-c(Cys-D-Ala-His-D-Phe-Arg-Trp-Cys)-NH₂;
Hydantoin(C(O)-(Val-Nle))-c(Cys-D-Ala-His-D-Phe-Arg-Trp-Cys)-NH₂;
Hydantoin(C(O)-(Leu-Nle))-c(Cys-D-Ala-His-D-Phe-Arg-Trp-Cys)-NH₂;
Hydantoin(C(O)-(Cha-Nle))-c(Cys-D-Ala-His-D-Phe-Arg-Trp-Cys)-NH₂;
Hydantoin(C(O)-(Aib-Nle))-c(Cys-D-Ala-His-D-Phe-Arg-Trp-Cys)-NH₂;
Hydantoin(C(O)-(Gly-Arg))-c(Cys-Glu-His-D-Phe-Arg-Trp-Cys)-NH₂;
Hydantoin(C(O)-(Gly-Arg))-c(Cys-Glu-His-D-2-Nal-Arg-Trp-Cys)-NH₂;
Hydantoin(C(O)-(Gly-Arg))-c(Cys-D-Ala-His-D-Phe-Arg-Trp-Cys)-NH₂;
Hydantoin(C(O)-(Gly-Arg))-c(Cys-D-Ala-His-D-2-Nal-Arg-Trp-Cys)-NH₂;
Hydantoin(C(O)-(Gly-D-Arg))-c(Cys-Glu-His-D-Phe-Arg-Trp-Cys)-NH₂;
Hydantoin(C(O)-(Gly-D-Arg))-c(Cys-D-Ala-His-D-Phe-Arg-Trp-Cys)-NH₂;
Hydantoin(C(O)-(Gly-D-Arg))-c(Cys-D-Ala-His-D-2-Nal-Arg-Trp-Cys)-NH₂;
Hydantoin(C(O)-(Nle-Ala))-c(Cys-Glu-His-D-Phe-Arg-Trp-Cys)-NH₂;
c[Hydantoin(C(O)-(Ala-Cys))-D-Ala-His-D-Phe-Arg-Trp-Cys]-NH₂,
c[Hydantoin(C(O)-(Nle-Cys))-D-Ala-His-D-Phe-Arg-Trp-Cys]-NH₂;
c[Hydantoin(C(O)-(D-Ala-Cys))-D-Ala-His-D-Phe-Arg-Trp-Cys]-NH₂;
c[Hydantoin(C(O)-(Aib-Cys))-D-Ala-His-D-Phe-Arg-Trp-Cys]-NH₂;
c[Hydantoin(C(O)-(Val-Cys))-D-Ala-His-D-Phe-Arg-Trp-Cys]-NH₂;
c[Hydantoin(C(O)-(Abu-Cys))-D-Ala-His-D-Phe-Arg-Trp-Cys]-NH₂;
c[Hydantoin(C(O)-(Leu-Cys))-D-Ala-His-D-Phe-Arg-Trp-Cys]-NH₂;
c[Hydantoin(C(O)-(Ile-Cys))-D-Ala-His-D-Phe-Arg-Trp-Cys]-NH₂;
c[Hydantoin(C(O)-(Cha-Cys))-D-Ala-His-D-Phe-Arg-Trp-Cys]-NH₂;
c[Hydantoin(C(O)-(A6c-Cys))-D-Ala-His-D-Phe-Arg-Trp-Cys]-NH₂;
c[Hydantoin(C(O)-(Phe-Cys))-D-Ala-His-D-Phe-Arg-Trp-Cys]-NH₂;
c[Hydantoin(C(O)-(Gly-Cys))-D-Ala-His-D-Phe-Arg-Trp-Cys]-NH₂;
c[Hydantoin(C(O)-(Gly-Cys))-Glu-His-D-Phe-Arg-Trp-Cys]-NH₂;
Ac-Tyr-Arg-c(Cys-D-Ala-His-D-Phe-Arg-Trp-Cys)-NH₂;
Ac-2-Nal-Arg-c(Cys-D-Ala-His-D-Phe-Arg-Trp-Cys)-NH₂;
Ac-1-Nal-Arg-c(Cys-D-Ala-His-D-Phe-Arg-Trp-Cys)-NH₂;
Ac-Phe-Arg-c(Cys-D-Ala-His-D-Phe-Arg-Trp-Cys)-NH₂;
Ac-Trp-Arg-c(Cys-D-Ala-His-D-Phe-Arg-Trp-Cys)-NH₂;
Ac-Pff-Arg-c(Cys-D-Ala-His-D-Phe-Arg-Trp-Cys)-NH₂;
H-His-Arg-c(Cys-D-Ala-His-D-Phe-Arg-Trp-Cys)-NH₂;
Ac-His-Arg-c(Cys-D-Ala-His-D-Phe-Arg-Trp-Cys)-NH₂; and
Ac-D-Arg-c(Cys-Glu-His-D-Phe-Arg-Trp-Cys)-NH₂
or a pharmaceutically acceptable salt thereof.

7. The method according to claim 6, wherein said compound is Ac-Arg-c(Cys-D-Ala-His-D-Phe-Arg-Trp-Cys)-NH₂ or a pharmaceutically acceptable salt thereof.

8. The method according to claim 6, wherein said compound is Hydantoin(C(O)-(Arg-Gly))-c(Cys-Glu-His-D-Phe-Arg-Trp-Cys)-NH₂ or a pharmaceutically acceptable salt thereof.

9. The method according to any one of claims 1-8, wherein said subject is obese, overweight, normal weight or lean.

10. The method according to claim 9, wherein said subject suffers from type II diabetes.

11. The method according to claim 1, wherein said peripheral administration is oral, subcutaneous, intraperitoneal, intramuscular, intravenous, rectal, transdermal or intranasal.

12. The method according to claim 11, wherein said administration is continuous, hourly, four times daily, three time daily, twice daily, once daily, once every other day, twice weekly, once weekly, once every two weeks, once a month, or once every two months.
